(19) United States
(12) Patent Application Publication
(10) Pub. No.: US 2022/0017597 A1
(43) Pub. Date: Jan. 20, 2022

(54) MULTIMERIC T-CELL MODULATORY POLYPEPTIDES AND METHODS OF USE THEREOF

(71) Applicant: Cue Biopharma, Inc., Cambridge, MA (US)

(72) Inventors: Ronald D. Seidel, III, Cambridge, MA (US); Rodolfo Chaparro, Cambridge, MA (US)

(21) Appl. No.: 17/490,600

(22) Filed: Sep. 30, 2021

Related U.S. Application Data
(63) Continuation of application No. PCT/US2020/037919, filed on Jun. 16, 2020.
(60) Provisional application No. 62/863,754, filed on Jun. 19, 2019.

(51) Int. Cl. C07K 14/74

(52) U.S. Cl. CPC C07K 14/70539; C07K 2319/40; A61K 38/00; C07K 2319/02; C07K 2319/33

(57) ABSTRACT

The present disclosure provides T-cell modulatory multimeric polypeptides that comprise an immunomodulatory N polypeptide, a MUC1 epitope-presenting peptide, and class I MHC polypeptides. A T-cell modulatory multimeric polypeptide is useful for modulating the activity of a T cell, and for modulating an immune response in an individual. The present disclosure provides a T-cell modulatory multimeric polypeptide (TMMP) comprising: a) a first polypeptide; and b) a second polypeptide, wherein the TMMP comprises a MUC1 peptide; a first major histocompatibility complex (MHC) polypeptide; a second MHC polypeptide; one or more immunomodulatory polypeptides; and optionally an immunoglobulin (Ig) Fc polypeptide or a non-lg scaffold.

### CROSS-REFERENCE

This application claims the benefit of U.S. Provisional Patent Application No. 62/863,754, filed Jun. 19, 2019, which application is incorporated herein by reference in its entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The TCR is specific for a given epitope; however, the costimulatory protein not epitope specific and instead is generally expressed on all T cells or on large T cell subsets.

### SUMMARY

The present disclosure provides T-cell modulatory multimeric polypeptides (TMMPs) that comprise an immunomodulatory polypeptide, class I HLA polypeptides (a class I HLA heavy chain polypeptide and a β2 microglobulin polypeptide), and a mucin-1 (MUC1) peptide that presents an epitope to a T-cell receptor. A TMMP is useful for modulating the activity of a T cell, and for modulating an immune response in an individual.

### BRIEF DESCRIPTION OF THE DRAWINGS

FIGS. 1A-1F are schematic depictions of various TMMPs of the present disclosure.

FIGS. 2A-2F are schematic depictions of various disulfide-linked TMMPs of the present disclosure.

FIGS. 3A-3G provide amino acid sequences of immunoglobulin Fc polypeptides. The sequences are set forth in: SEQ ID NOs: 19-30.

FIG. 4 provides a multiple amino acid sequence alignment of beta-2 microglobulin (β2M) precursors (i.e., including the leader sequence) from Homo sapiens (NP_004039.1; SEQ ID NO:31), Pan troglodytes (NP_001009066.1; SEQ ID NO:31), Macaca mulatta (NP_001040602.1; SEQ ID NO:32), Bos taurus (NP_776318.1; SEQ ID NO:33) and Mus musculus (NP_033865.2; SEQ ID NO:34). Amino acids 1-20 are a signal peptide.

FIGS. 5A-5C provide amino acid sequences of full-length human HLA heavy chains of alleles A*0101 (SEQ ID NO: 35), A*1101 (SEQ ID NO: 36), A*2402 (SEQ ID NO: 37), and A*3303 (SEQ ID NO: 38) (FIG. 7A); full-length human HLA heavy chain of allele B*0702 (SEQ ID NO: 39) (FIG. 7B); and a full-length human HLA-C heavy chain (SEQ ID NO: 40) (FIG. 7C).

FIG. 6 provides an alignment of eleven mature MHC class I heavy chain amino acid sequences without their leader sequences, transmembrane domains, and intracellular domains. The sequences are set forth from top to bottom as follows: SEQ ID NOs: 41-51.

FIGS. 7A-7B provide an alignment of HLA-A heavy chain amino acid sequences (FIG. 7A; SEQ ID NO: 52-60, respectively) and a consensus sequence (FIG. 7B: 61).

FIGS. 8A-8B provide an alignment of HLA-B heavy chain amino acid sequences (FIG. 8A; SEQ ID NOs: 62-68, respectively) and a consensus sequence (FIG. 8B; SEQ ID NO: 69).

FIGS. 9A-9B provide an alignment of HLA-C heavy chain amino acid sequences (FIG. 9A; SEQ ID NOs: 70-78, respectively) and a consensus sequence (FIG. 9B; SEQ ID NO: 79).

FIG. 10 provides a consensus amino acid sequence for each of HLA-E, -F, and -G heavy chains (SEQ ID NOs: 80-82, respectively). Variable amino acid (aa) positions are indicated as “X” residues sequentially numbered; the locations of amino acids 84, 139, and 236 are double underlined.

FIG. 11 provides an alignment of consensus amino acid sequences for HLA-A (SEQ ID NO: 83), -B (SEQ ID NO: 84), -C(SEQ ID NO: 85), -E (SEQ ID NO: 86), -F (SEQ ID NO: 87), and -G (SEQ ID NO: 88).

FIG. 12A-12D provide schematic depictions of multiple disulfide-linked TMMP of the present disclosure.

FIGS. 13A and 13B provide amino acid sequences of examples of polypeptides suitable for inclusion in a TMMP of the present disclosure. The sequences are set forth in SEQ ID NOs: 594-595, respectively.

FIG. 14A-14L provide amino acid sequences of examples of polypeptides suitable for inclusion in a TMMP of the present disclosure. The sequences are set forth in SEQ ID NOs: 596-607, respectively.

FIG. 15A-15C provide schematic depictions of examples of configurations of disulfide-linked TMMPs of the present disclosure.

FIG. 16 provide schematic depictions of examples of positions of immunomodulatory polypeptides in TMMPs of the present disclosure.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “co-stimulatory polypeptide”), as used herein, includes a polypeptide on an antigen presenting cell (APC) (e.g., a dendritic cell, a B cell, and the like) that specifically binds a cognate co-immunomodulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. An immunomodulatory polypeptide can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3.

As noted above, an “immunomodulatory polypeptide” (also referred to herein as a “MOD”) specifically binds a cognate co-immunomodulatory polypeptide on a T cell.

An “immunomodulatory domain” (“MOD”) of a TMMP of the present disclosure binds a cognate co-immunomodulatory polypeptide, which may be present on a target T cell.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g. with reference to binding of a TMMP to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹²M, less than 10⁻¹³ M, less than 10⁻¹⁴ M, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ M, 10⁻⁸ M, 5×10⁻⁸ M, 10⁻⁹ M, and greater. “Non-specific binding” generally refers to binding (e.g., the binding of a ligand to a moiety other than its designated binding site or receptor) with an affinity of less than about 10⁻⁷ M (e.g., binding with an affinity of 10⁻⁶ M, 10⁻⁵ M, 10⁻⁴ M). However, in some contexts, e.g., binding between a TCR and a peptide/MHC complex, “specific binding” can be in the range of from 1 μM to 100 μM, or from 100 μM to 1 mM. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., U.S. Pat. No. 4,816,567; European Patent No. 0,125,023 B1; U.S. Pat. No. 4,816,397; European Patent No. 0,120,694 B1; WO 86/01533; European Patent No. 0,194,276 B1; U.S. Pat. No. 5,225,539; European Patent No. 0,239,400 B1; and European Patent Application No. 0,519,596 A1. See also, U.S. Pat. Nos. 4,946,778; 5,476,786; and Bird et al. (1988) Science 242:423, regarding single chain antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}—V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 2 as a comparison.

TABLE 2
CDR Definitions

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

As used herein, the term “framework,” when used in reference to an antibody variable region, is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to a “T-cell modulatory multimeric polypeptide” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides T-cell modulatory multimeric polypeptides that comprise an immunomodulatory polypeptide and that comprise an epitope-presenting peptide. A TMMP is useful for modulating the activity of a T cell, and for modulating an immune response in an individual.

### T-Cell Modulatory Multimeric Polypeptides

The present disclosure provides a T-cell modulatory multimeric polypeptide (TMMP) comprising: a) a first polypeptide; and b) a second polypeptide, wherein the TMMP comprises a MUC1 peptide; a first major histocompatibility complex (MHC) polypeptide; a second MHC polypeptide; one or more immunomodulatory polypeptides; and optionally an immunoglobulin (Ig) Fc polypeptide or a non-Ig scaffold. As used herein, the term “MUC1 peptide” means a peptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) that present a MUC1 epitope to a T-cell receptor (TCR) when the MUC1 peptide is bound to an MHC complex. As used herein, the term “MUC1 epitope” means an epitope found on a MUC1 protein. As used herein, the terms “MUC1” and “MUC1 protein” are synonymous and mean a protein having an amino acid sequence of one of the following: (i) the mature MUC1 protein (discussed below); (ii) components of the mature MUC-1 protein, e.g., MUC1-N, MUC-1 C, and the extracellular region, transmembrane domain, and cytoplasmic tail of MUC1-C (all discussed below); and (iii) variants of (i) and (ii) that occur in human cancers, including, e.g., mutated forms, splice variants, isoforms, hypoglycosylated forms, cleaved forms, and the like. As used herein, the term “MUC1 polypeptide” means a polypeptide having a sequence of amino acids found in all or a part of a MUC1 protein, or where specified, a polypeptide having at least an 80% sequence identity to a sequence of amino acids found in all or a part of a MUC1 protein.

MUC1 (also known as tumor-associated epithelial membrane antigen, tumor-associated epithelial mucin, CD227 antigen, breast carcinoma-associated antigen DF3, peanut-reactive urinary mucin, polymorphic epithelial mucin, carcinoma-associated mucin, Krebs von den Lungen-6, episialin, PEM, H23Ag, EMA, MUC-1/SEC, ADMCKD1, MAM6, cancer antigen 15-3 (CA15-3), and MCA) is a polymorphic transmembrane glycoprotein expressed on the apical surfaces of simple and glandular epithelia. Nath and Mukherjee (2014) Trends Mol. Med. 20:332. The MUC1 gene encodes a single polypeptide chain precursor that includes a signal peptide sequence. Immediately after translation the signal peptide sequence is removed and the remaining portion of the MUC1 precursor is further cleaved into two peptide fragments: the longer N-terminal subunit (MUC1-N or MUC1a) and the shorter C-terminal subunit (MUC1-C or MUC16). The mature MUC1 comprises a MUC1-N and a MUC1-C associated through stable hydrogen bonds. MUC1-N, which is an extracellular domain, contains 25 to 125 variable number tandem repeats (VNTR) of 20 amino acid residues. MUC1-C contains a short extracellular region (approximately 53 amino acids), a transmembrane domain (approximately 28 amino acid), and a cytoplasmic tail (approximately 72 amino acids). The cytoplasmic tail of MUC1 (MUC1-CT) contains highly conserved serine and tyrosine residues that are phosphorylated by growth factor receptors and intracellular kinases. Human MUC1 exists in multiple isoforms resulting from different types of MUC1 RNA alternative splicing. The amino acid sequence of full length human MUC1 isoform 1 protein precursor (isoform 1, Uniprot P15941-1) is provided below. At least 16 other isoforms of human MUC-1 have been reported so far (Uniprot P15941-2 through P15941-17), which include various insertions, deletions, or substitutions as compared to the sequence of isoform 1. These isoforms are known as isoform 2, 3, 4, 5, 6, Y, 8, 9, F, Y-LSP, S2, M6, ZD, T10, E2, and J13 (Uniprot P15941-2 through P15941-17, respectively). The full length human MUC1 isoform 1 precursor protein consists of 1255 amino acids, which includes a signal peptide sequence at amino acids 1-23. The MUC1-N and MUC1-C domains of the mature MUC1 protein consist of amino acids 24-1097 and 1098-1255, respectively.

The present disclosure provides a TMMP, wherein the TMMP is a heterodimer comprising: a) a first polypeptide comprising a first MHC polypeptide; and b) a second polypeptide comprising a second MHC polypeptide, wherein the first polypeptide or the second polypeptide comprises a MUC1 peptide; wherein the first polypeptide and/or the second polypeptide comprises one or more immunomodulatory polypeptides that can be the same or different; and optionally an Ig F c polypeptide or a non-Ig scaffold. A TMMP of the present disclosure is also referred to herein as a “multimeric polypeptide of the present disclosure” or a “synTac.”

The present disclosure provides a TMMP comprising a heterodimeric polypeptide comprising: a) a first polypeptide comprising: i) a MUC1 peptide; and ii) a first MHC polypeptide; b) a second polypeptide comprising a second MHC polypeptide; and c) at least one immunomodulatory polypeptide, where the first and/or the second polypeptide comprises the at least one (i.e., one or more) immunomodulatory polypeptide. Optionally, the first or the second polypeptide comprises an Ig Fc polypeptide or a non-Ig scaffold. At least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide. The MUC1 peptide present in a TMMP of the present disclosure binds to a T-cell receptor (TCR) on a T cell with an affinity of at least 100 μM (e.g., at least 10 μM, at least 1 μM, at least 100 nM, at least 10 nM, or at least 1 nM). A TMMP of the present disclosure binds to a first T cell with an affinity that is at least 25% higher than the affinity with which the TMMP binds a second T cell, where the first T cell expresses on its surface the cognate co-immunomodulatory polypeptide and a TCR that binds the MUC1 peptide/MHC complex with an affinity of at least 100 μM, and where the second T cell expresses on its surface the cognate co-immunomodulatory polypeptide but does not express on its surface a TCR that binds the MUC1 peptide/MHC complex with an affinity of at least 100 μM (e.g., at least 10 μM, at least 1 μM, at least 100 nM, at least 10 nM, or at least 1 nM).

The present disclosure provides a TMMP, wherein the TMMP is:

A) a heterodimer comprising: a) a first polypeptide comprising a first MHC polypeptide; and b) a second polypeptide comprising a second MHC polypeptide, wherein the first polypeptide or the second polypeptide comprises a MUC1 peptide; wherein the first polypeptide and/or the second polypeptide comprises one or more immunomodulatory polypeptides that can be the same or different, and wherein at least one of the one or more immunomodulatory polypeptides may be a wild-type immunomodulatory polypeptide or a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and wherein the first polypeptide or the second polypeptide optionally comprises an Ig Fc polypeptide or a non-Ig scaffold; or

B) a heterodimer comprising: a) a first polypeptide comprising a first MHC polypeptide; and b) a second polypeptide comprising a second MHC polypeptide, wherein the first polypeptide or the second polypeptide comprises a MUC1 peptide; wherein the first polypeptide and/or the second polypeptide comprises one or more immunomodulatory polypeptides that can be the same or different,

wherein at least one of the one or more immunomodulatory polypeptides is a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide,

wherein at least one of the one or more immunomodulatory domains is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide, and wherein the MUC1 peptide/MHC complex binds to a TCR on a T cell with an affinity of at least 10⁻⁷ M, such that: i) the TMMP polypeptide binds to a first T cell with an affinity that is at least 25% higher than the affinity with which the TMMP binds a second T cell, wherein the first T cell expresses on its surface the cognate co-immunomodulatory polypeptide and a TCR that binds the MUC1 peptide/MHC complex with an affinity of at least 10⁻⁷ M, and wherein the second T cell expresses on its surface the cognate co-immunomodulatory polypeptide but does not express on its surface a TCR that binds the MUC1 peptide/MHC complex with an affinity of at least 10⁻⁷ M; and/or ii) the ratio of the binding affinity of a control TMMP, wherein the control comprises a wild-type immunomodulatory polypeptide, to a cognate co-immunomodulatory polypeptide to the binding affinity of the TMMP comprising a variant of the wild-type immunomodulatory polypeptide to the cognate co-immunomodulatory polypeptide, when measured by bio-layer interferometry, is in a range of from 1.5:1 to 10⁶:1; and wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and

wherein the first polypeptide or the second polypeptide optionally comprises an Ig Fc polypeptide or a non-Ig scaffold; or

C) a heterodimer comprising: a) a first polypeptide comprising, in order from N-terminus to C-terminus: i) a MUC1 peptide; ii) a first MHC polypeptide; and b) a second polypeptide comprising, in order from N-terminus to C-terminus: i) a second MHC polypeptide; and ii) optionally an immunoglobulin (Ig) Fc polypeptide or a non-Ig scaffold, wherein the TMMP comprises one or more immunomodulatory domains that can be the same or different, wherein at least one of the one or more immunomodulatory domain is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide, and wherein at least one of the one or more immunomodulatory domains may be a wild-type immunomodulatory polypeptide or a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and

optionally wherein at least one of the one or more immunomodulatory domains is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide, and wherein the MUC1 peptide/MHC complex binds to a TCR on a T cell with an affinity of at least 10⁻⁷ M, such that: i) the TMMP binds to a first T cell with an affinity that is at least 25% higher than the affinity with which the TMMP binds a second T cell, wherein the first T cell expresses on its surface the cognate co-immunomodulatory polypeptide and a TCR that binds the MUC1 peptide/MHC complex with an affinity of at least 10⁻⁷ M, and wherein the second T cell expresses on its surface the cognate co-immunomodulatory polypeptide but does not express on its surface a TCR that binds the MUC1 peptide/MHC complex with an affinity of at least 10⁻⁷ M; and/or ii) the ratio of the binding affinity of a control TMMP, wherein the control comprises a wild-type immunomodulatory polypeptide, to a cognate co-immunomodulatory polypeptide to the binding affinity of the TMMP comprising a variant of the wild-type immunomodulatory polypeptide to the cognate co-immunomodulatory polypeptide, when measured by bio-layer interferometry, is in a range of from 1.5:1 to 10⁶:1; and wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide.

The present disclosure provides a TMMP comprising: a) a first polypeptide comprising, in order from N-terminus to C-terminus: i) a MUC1 peptide; ii) a first MHC polypeptide; and b) a second polypeptide comprising, in order from N-terminus to C-terminus: i) a second MHC polypeptide; and ii) optionally an Ig Fc polypeptide or a non-Ig scaffold. A TMMP of the present disclosure comprises one or more immunomodulatory polypeptides, wherein at least one of the one or more immunomodulatory polypeptides is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide. At least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide. The MUC1 peptide/MHC complex present in a TMMP of the present disclosure binds to a T-cell receptor (TCR) on a T cell with an affinity of at least 100 μM (e.g., at least 10 μM, at least 1 μM, at least 100 nM, at least 10 nM, or at least 1 nM). A TMMP of the present disclosure binds to a first T cell with an affinity that is at least 25% higher than the affinity with which the TMMP binds a second T cell, where the first T cell expresses on its surface the cognate co-immunomodulatory polypeptide and a TCR that binds the MUC1 peptide/MHC complex with an affinity of at least 100 μM, and where the second T cell expresses on its surface the cognate co-immunomodulatory polypeptide but does not express on its surface a TCR that binds the MUC1 peptide/MHC complex with an affinity of at least 100 μM (e.g., at least 10 μM, at least 1 μM, at least 100 nM, at least 10 nM, or at least 1 nM).

In some cases, the MUC1 peptide/MHC complex present in a TMMP of the present disclosure binds to a TCR on a T cell with an affinity of from about 10⁻⁴ M to about 5×10⁻⁴ M, from about 5×10⁻⁴ M to about 10⁻⁵ M, from about 10⁻⁵ M to 5×10⁻⁵ M, from about 5×10⁻⁵ M to 10⁻⁶ M, from about 10⁻⁶ M to about 5×10⁻⁶ M, from about 5×10⁻⁶ M to about 10⁻⁷ M, from about 10⁻⁷ M to about 5×10⁻⁷ M, from about 5×10⁻⁷ M to about 10⁻⁸ M, or from about 10⁻⁸ M to about 10⁻⁹ M. Expressed another way, in some cases, the MUC1 peptide/MHC complex present in a TMMP of the present disclosure binds to a TCR on a T cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, from about 75 μM to about 100 μM.

In some cases, an immunomodulatory polypeptide present in a TMMP of the present disclosure binds to its cognate co-immunomodulatory polypeptide with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type immunomodulatory polypeptide for the cognate co-immunomodulatory polypeptide.

In some cases, a variant immunomodulatory polypeptide present in a TMMP of the present disclosure has a binding affinity for a cognate co-immunomodulatory polypeptide that is from 1 nM to 100 nM, or from 100 nM to 100 μM. For example, in some cases, a variant immunomodulatory polypeptide present in a TMMP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant immunomodulatory polypeptide present in a TMMP of the present disclosure has a binding affinity for a cognate co-immunomodulatory polypeptide that is from about 1 nM to about 5 nM, from about 5 nM to about 10 nM, from about 10 nM to about 50 nM, from about 50 nM to about 100 nM.

The combination of the reduced affinity of the immunomodulatory polypeptide for its cognate co-immunomodulatory polypeptide, and the affinity of the MUC1 peptide for a TCR, provides for enhanced selectivity of a TMMP of the present disclosure. For example, a TMMP of the present disclosure binds selectively to a first T cell that displays both: i) a TCR specific for the MUC1 peptide present in the TMMP; and ii) a co-immunomodulatory polypeptide that binds to the immunomodulatory polypeptide present in the TMMP, compared to binding to a second T cell that displays: i) a TCR specific for an epitope other than the epitope present in the TMMP; and ii) a co-immunomodulatory polypeptide that binds to the immunomodulatory polypeptide present in the TMMP. For example, a TMMP of the present disclosure binds to the first T cell with an affinity that is at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, at least 15-fold, at least 20-fold, at least 25-fold, at least 50-fold, at least 100-fold, or more than 100-fold, higher than the affinity to which it binds the second T cell.

In some cases, a TMMP of the present disclosure, when administered to an individual in need thereof, induces both an epitope-specific T cell response and an epitope non-specific T cell response. In other words, in some cases, a TMMP of the present disclosure, when administered to an individual in need thereof, induces an epitope-specific T cell response by modulating the activity of a first T cell that displays both: i) a TCR specific for the MUC1 epitope present in the TMMP; ii) a co-immunomodulatory polypeptide that binds to the immunomodulatory polypeptide present in the TMMP; and induces an epitope non-specific T cell response by modulating the activity of a second T cell that displays: i) a TCR specific for an epitope other than the MUC1 epitope present in the TMMP; and ii) a co-immunomodulatory polypeptide that binds to the immunomodulatory polypeptide present in the TMMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; iv) inhibiting activity of an autoreactive T cell; and the like.

The combination of the reduced affinity of the immunomodulatory polypeptide for its cognate co-immunomodulatory polypeptide, and the affinity of the MUC1 epitope for a TCR, provides for enhanced selectivity of a TMMP of the present disclosure. Thus, for example, a TMMP of the present disclosure binds with higher avidity to a first T cell that displays both: i) a TCR specific for the MUC1 epitope present in the TMMP; and ii) a co-immunomodulatory polypeptide that binds to the immunomodulatory polypeptide present in the TMMP, compared to the avidity to which it binds to a second T cell that displays: i) a TCR specific for an epitope other than the epitope present in the TMMP; and ii) a co-immunomodulatory polypeptide that binds to the immunomodulatory polypeptide present in the TMMP.

Binding affinity between an immunomodulatory polypeptide and its cognate co-immunomodulatory polypeptide can be determined by bio-layer interferometry (BLI) using purified immunomodulatory polypeptide and purified cognate co-immunomodulatory polypeptide. Binding affinity between a TMMP and its cognate co-immunomodulatory polypeptide can be determined by BLI using purified TMMP and the cognate co-immunomodulatory polypeptide. BLI methods are well known to those skilled in the art. See, e.g., Lad et al. (2015) J. Biomol. Screen. 20(4):498-507; and Shah and Duncan (2014) J. Vis. Exp. 18:e51383.

A BLI assay can be carried out using an Octet RED 96 (Pal FortéBio) instrument, or a similar instrument, as follows. A TMMP (e.g., a TMMP of the present disclosure; a control TMMP (where a control TMMP comprises a wild-type immunomodulatory polypeptide)) is immobilized onto an insoluble support (a “biosensor”). The immobilized TMMP is the “target.” Immobilization can be effected by immobilizing a capture antibody onto the insoluble support, where the capture antibody immobilizes the TMMP. For example, immobilization can be effected by immobilizing anti-Fc (e.g., anti-human IgG Fc) antibodies onto the insoluble support, where the immobilized anti-Fc antibodies bind to and immobilize the TMMP (where the TMMP comprises an IgFc polypeptide). A co-immunomodulatory polypeptide is applied, at several different concentrations, to the immobilized TMMP, and the instrument's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized TMMP is conducted at 30° C. As a positive control for binding affinity, an anti-MHC Class I monoclonal antibody can be used. For example, anti-HLA Class I monoclonal antibody W6/32 (American Type Culture Collection No. HB-95; Parham et al. (1979) J. Immunol. 123:342), which has a K_{D} of 7 nM, can be used. A standard curve can be generated using serial dilutions of the anti-MHC Class I monoclonal antibody. The co-immunomodulatory polypeptide, or the anti-MHC Class I mAb, is the “analyte.” BLI analyzes the interference pattern of white light reflected from two surfaces: i) from the immobilized polypeptide (“target”); and ii) an internal reference layer. A change in the number of molecules (“analyte”; e.g., co-immunomodulatory polypeptide; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/ₐ) gives rise to the affinity constant K_{D}.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

Unless otherwise stated herein, the affinity of a TMMP of the present disclosure for a cognate co-immunomodulatory polypeptide, or the affinity of a control TMMP (where a control TMMP comprises a wild-type immunomodulatory polypeptide) for a cognate co-immunomodulatory polypeptide, is determined using BLI, as described above.

In some cases, the ratio of: i) the binding affinity of a control TMMP (where the control comprises a wild-type immunomodulatory polypeptide) to a cognate co-immunomodulatory polypeptide to ii) the binding affinity of a TMMP of the present disclosure comprising a variant of the wild-type immunomodulatory polypeptide to the cognate co-immunomodulatory polypeptide, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control TMMP (where the control comprises a wild-type immunomodulatory polypeptide) to a cognate co-immunomodulatory polypeptide to ii) the binding affinity of a TMMP of the present disclosure comprising a variant of the wild-type immunomodulatory polypeptide to the cognate co-immunomodulatory polypeptide, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As an example, where a control TMMP comprises a wild-type IL-2 polypeptide, and where a TMMP of the present disclosure comprises a variant IL-2 polypeptide (comprising from 1 to 10 amino acid substitutions relative to the amino acid sequence of the wild-type IL-2 polypeptide) as the immunomodulatory polypeptide, the ratio of: i) the binding affinity of the control TMMP to an IL-2 receptor (i.e., the cognate co-immunomodulatory polypeptide) to ii) the binding affinity of the TMMP of the present disclosure to the IL-2 receptor, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, where a control TMMP comprises a wild-type IL-2 polypeptide, and where a TMMP of the present disclosure comprises a variant IL-2 polypeptide (comprising from 1 to 10 amino acid substitutions relative to the amino acid sequence of the wild-type IL-2 polypeptide) as the immunomodulatory polypeptide, the ratio of: i) the binding affinity of the control TMMP to an IL-2 receptor (i.e., the cognate co-immunomodulatory polypeptide) to ii) the binding affinity of the TMMP of the present disclosure to the IL-2 receptor, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As another example, where a control TMMP comprises a wild-type PD-L1 polypeptide, and where a TMMP of the present disclosure comprises a variant PD-L1 polypeptide (comprising from 1 to 10 amino acid substitutions relative to the amino acid sequence of the wild-type PD-L1 polypeptide) as the immunomodulatory polypeptide, the ratio of: i) the binding affinity of the control TMMP to a PD-1 polypeptide (i.e., the cognate co-immunomodulatory polypeptide) to ii) the binding affinity of the TMMP of the present disclosure to the PD-1 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type CD80 polypeptide, and where a TMMP of the present disclosure comprises a variant CD80 polypeptide (comprising from 1 to 10 amino acid substitutions relative to the amino acid sequence of the wild-type CD80 polypeptide) as the immunomodulatory polypeptide, the ratio of: i) the binding affinity of the control TMMP to a CTLA4 polypeptide (i.e., the cognate co-immunomodulatory polypeptide) to ii) the binding affinity of the TMMP of the present disclosure to the CTLA4 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type CD80 polypeptide, and where a TMMP of the present disclosure comprises a variant CD80 polypeptide (comprising from 1 to 10 amino acid substitutions relative to the amino acid sequence of the wild-type CD80 polypeptide) as the immunomodulatory polypeptide, the ratio of: i) the binding affinity of the control TMMP to a CD28 polypeptide (i.e., the cognate co-immunomodulatory polypeptide) to ii) the binding affinity of the TMMP of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type 4-1BBL polypeptide, and where a TMMP of the present disclosure comprises a variant 4-1BBL polypeptide (comprising from 1 to 10 amino acid substitutions relative to the amino acid sequence of the wild-type 4-1BBL polypeptide) as the immunomodulatory polypeptide, the ratio of: i) the binding affinity of the control TMMP to a 4-1BB polypeptide (i.e., the cognate co-immunomodulatory polypeptide) to ii) the binding affinity of the TMMP of the present disclosure to the 4-1BB polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type CD86 polypeptide, and where a TMMP of the present disclosure comprises a variant CD86 polypeptide (comprising from 1 to 10 amino acid substitutions relative to the amino acid sequence of the wild-type CD86 polypeptide) as the immunomodulatory polypeptide, the ratio of: i) the binding affinity of the control TMMP to a CD28 polypeptide (i.e., the cognate co-immunomodulatory polypeptide) to ii) the binding affinity of the TMMP of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

Binding affinity of a TMMP of the present disclosure to a target T cell can be measured in the following manner: A) contacting a TMMP of the present disclosure with a target T-cell expressing on its surface: i) a cognate co-immunomodulatory polypeptide that binds the parental wild-type immunomodulatory polypeptide; and ii) a T-cell receptor that binds to the epitope, where the TMMP comprises an epitope tag, such that the TMMP binds to the target T-cell; B) contacting the target T-cell-bound TMMP with a fluorescently labeled binding agent (e.g., a fluorescently labeled antibody) that binds to the epitope tag, generating a TMMP/target T-cell/binding agent complex; C) measuring the mean fluorescence intensity (MFI) of the TMMP/target T-cell/binding agent complex using flow cytometry. The epitope tag can be, e.g., a FLAG tag, a hemagglutinin tag, a c-myc tag, a poly(histidine) tag, etc. The MFI measured over a range of concentrations of the TMMP library member provides a measure of the affinity. The MFI measured over a range of concentrations of the TMMP library member provides a half maximal effective concentration (EC₅₀) of the TMMP. In some cases, the EC₅₀ of a TMMP of the present disclosure for a target T cell is in the nM range; and the EC₅₀ of the TMMP for a control T cell (where a control T cell expresses on its surface: i) a cognate co-immunomodulatory polypeptide that binds the parental wild-type immunomodulatory polypeptide; and ii) a T-cell receptor that does not bind to the epitope present in the TMMP) is in the M range. In some cases, the ratio of the EC₅₀ of a TMMP of the present disclosure for a control T cell to the EC₅₀ of the TMMP for a target T cell is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at lease 10⁵:1, or at least 10⁶:1. The ratio of the EC₅₀ of a TMMP of the present disclosure for a control T cell to the EC₅₀ of the TMMP for a target T cell is an expression of the selectivity of the TMMP.

In some cases, when measured as described in the preceding paragraph, a TMMP of the present disclosure exhibits selective binding to target T-cell, compared to binding of the TMMP library member to a control T cell that comprises: i) the cognate co-immunomodulatory polypeptide that binds the parental wild-type immunomodulatory polypeptide; and ii) a T-cell receptor that binds to an epitope other than the epitope present in the TMMP library member.

### Dimerized TMMPs

A TMMP of the present disclosure can be dimerized; i.e., the present disclosure provides a multimeric polypeptide comprising a dimer of a TMMP of the present disclosure. Thus, the present disclosure provides a TMMP comprising: A) a first heterodimer comprising: a) a first polypeptide comprising: i) a MUC1 peptide; and ii) a first major histocompatibility complex (MHC) polypeptide; and b) a second polypeptide comprising: i) a second MHC polypeptide, wherein the first heterodimer comprises one or more immunomodulatory polypeptides; and B) a second heterodimer comprising: a) a first polypeptide comprising: i) a MUC1 peptide; and ii) a first MHC polypeptide; and b) a second polypeptide comprising: i) a second MHC polypeptide, wherein the second heterodimer comprises one or more immunomodulatory polypeptides, and wherein the first heterodimer and the second heterodimer are covalently linked to one another. In some cases, the two TMMPs are identical to one another in amino acid sequence. In some cases, the first heterodimer and the second heterodimer are covalently linked to one another via a C-terminal region of the second polypeptide of the first heterodimer and a C-terminal region of the second polypeptide of the second heterodimer. In some cases, first heterodimer and the second heterodimer are covalently linked to one another via the C-terminal amino acid of the second polypeptide of the first heterodimer and the C-terminal region of the second polypeptide of the second heterodimer; for example, in some cases, the C-terminal amino acid of the second polypeptide of the first heterodimer and the C-terminal region of the second polypeptide of the second heterodimer are linked to one another, either directly or via a linker. The linker can be a peptide linker. The peptide linker can have a length of from 1 amino acid to 200 amino acids (e.g., from 1 amino acid (aa) to 5 aa, from 5 aa to 10 aa, from 10 aa to 25 aa, from 25 aa to 50 aa, from 50 aa to 100 aa, from 100 aa to 150 aa, or from 150 aa to 200 aa). In some cases, the peptide epitope of the first heterodimer and the peptide epitope of the second heterodimer comprise the same amino acid sequence. In some cases, the first MHC polypeptide of the first and the second heterodimer is an MHC Class I β2-microglobulin, and wherein the second MHC polypeptide of the first and the second heterodimer is an MHC Class I heavy chain. In some cases, the immunomodulatory polypeptide of the first heterodimer and the immunomodulatory polypeptide of the second heterodimer comprise the same amino acid sequence. In some cases, the immunomodulatory polypeptide of the first heterodimer and the immunomodulatory polypeptide of the second heterodimer are variant immunomodulatory polypeptides that comprise from 1 to 10 amino acid substitutions compared to a corresponding parental wild-type immunomodulatory polypeptide, and wherein the from 1 to 10 amino acid substitutions result in reduced affinity binding of the variant immunomodulatory polypeptide to a cognate co-immunomodulatory polypeptide. In some cases, the immunomodulatory polypeptide of the first heterodimer and the immunomodulatory polypeptide of the second heterodimer are each independently selected from the group consisting of IL-2, 4-1BBL, PD-L1, CD80, CD86, ICOS-L, OX-40L, FasL, JAG1 (CD339), TGFβ, CD70, and ICAM. Examples, of suitable MHC polypeptides, immunomodulatory polypeptides, and peptide epitopes are described below.

### MHC Polypeptides

As noted above, a TMMP of the present disclosure includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC class I heavy chain).

In some cases, the first MHC polypeptide is an MHC Class I β2M (β2M) polypeptide, and the second MHC polypeptide is an MHC Class I heavy chain (H chain) (“MHC-H”)). In other instances, the first MHC polypeptide is an MHC Class I heavy chain polypeptide; and the second MHC polypeptide is a β2M polypeptide. In some cases, both the β2M and MHC-H chain are of human origin; i.e., the MHC-H chain is an HLA heavy chain, or a variant thereof. Unless expressly stated otherwise, a TMMP of the present disclosure does not include membrane anchoring domains (transmembrane regions) of an MHC Class I heavy chain, or a part of MHC Class I heavy chain sufficient to anchor the resulting TMMP to a cell (e.g., eukaryotic cell such as a mammalian cell) in which it is expressed. In some cases, the MHC Class I heavy chain present in a TMMP of the present disclosure does not include a signal peptide, a transmembrane domain, or an intracellular domain (cytoplasmic tail) associated with a native MHC Class I heavy chain. Thus, e.g., in some cases, the MHC Class I heavy chain present in a TMMP of the present disclosure includes only the α1, α2, and α3 domains of an MHC Class I heavy chain. In some cases, the MHC Class I heavy chain present in a TMMP of the present disclosure has a length of from about 270 amino acids (aa) to about 290 aa. In some cases, the MHC Class I heavy chain present in a TMMP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC polypeptide of a TMMP is a human MHC polypeptide, where human MHC polypeptides are also referred to as “human leukocyte antigen” (“HLA”) polypeptides. In some cases, an MHC polypeptide of a TMMP is a Class I HLA polypeptide, e.g., a β2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

### MHC Class I Heavy Chains

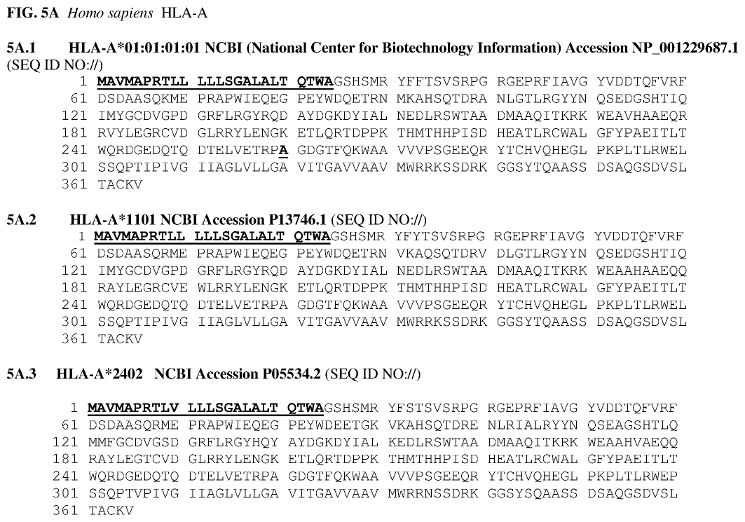

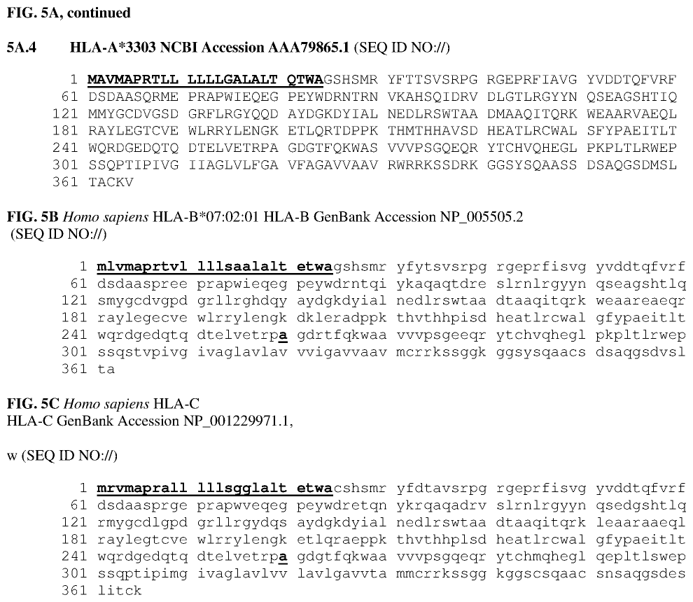

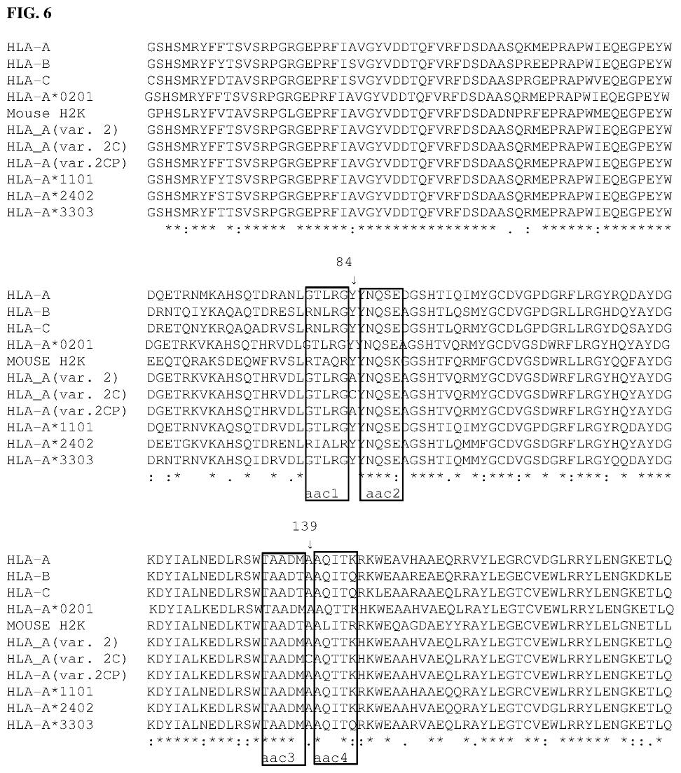

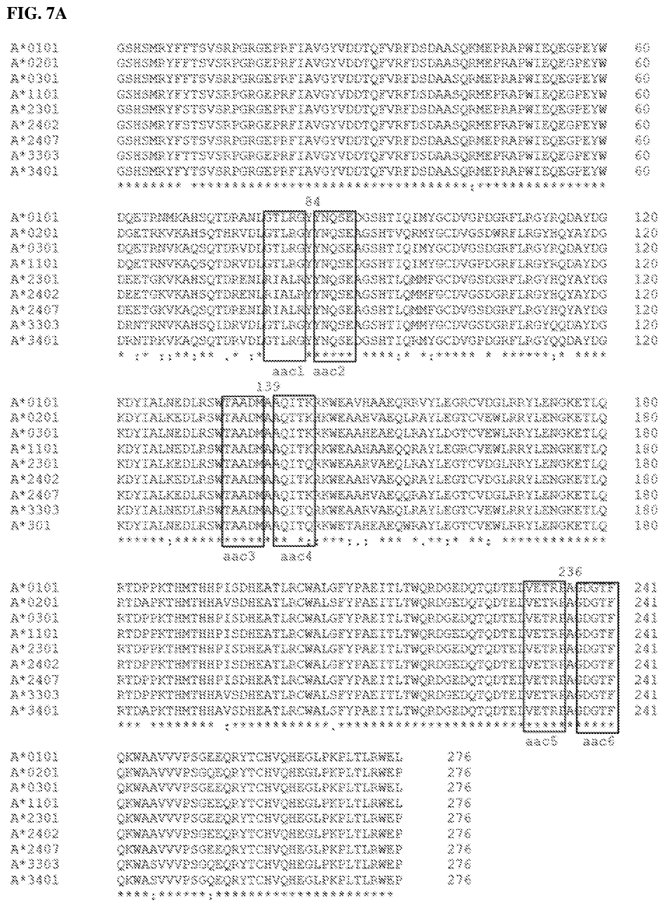

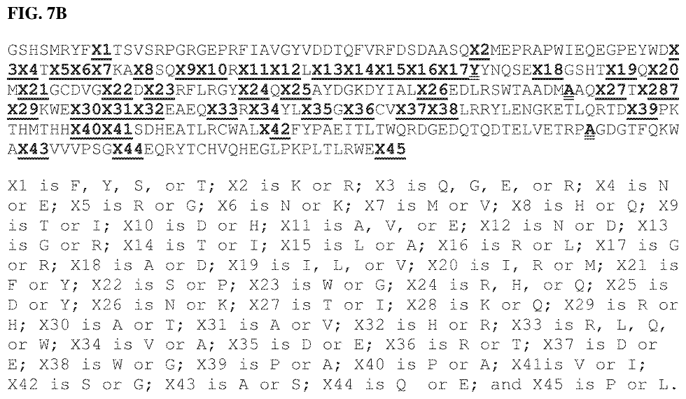

In some cases, an MHC Class I heavy chain polypeptide present in a TMMP of the present disclosure comprises an amino acid sequence having at least 75%, at least 80%, at least 85%, at least 90%, at least 95%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 7-13. In some cases, the MHC Class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa. In some cases, an MHC Class I heavy chain polypeptide present in a TMMP of the present disclosure comprises 1-30, 1-5, 5-10, 10-15, 15-20, 20-25 or 25-30 amino acid insertions, deletions, and/or substitutions (in addition to those locations indicated as being variable in the heavy chain consensus sequences) of any one of the amino acid sequences depicted in FIGS. 7-13. In some cases, the MHC Class I heavy chain does not include transmembrane or cytoplasmic domains. As an example, a MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to amino acids 25-300 (lacking all, or substantially all, of the leader, transmembrane and cytoplasmic sequence) or amino acids 25-365 (lacking the leader) of a human HLA-A heavy chain polypeptides depicted in any one of FIGS. 5A, 5B, and 5C.

FIGS. 5A, 5B and 5C provide amino acid sequences of human leukocyte antigen (HLA) Class I heavy chain polypeptides. Signal sequences, amino acids 1-24, are bolded and underlined. FIG. 5A entry: 3A.1 is the HLA-A heavy chain (HLA-A*01:01:01:01 or A*0101) (NCBI accession NP_001229687.1), SEQ ID NO: 35; entry 3A.2 is from HLA-A*1101 SEQ ID NO: 36; entry 3A.3 is from HLA-A*2402 SEQ ID NO: 37 and entry 3A.4 is from HLA-A*3303 SEQ ID NO: 38. FIG. 5B provides the sequence HLA-B*07:02:01 (HLA-B*0702) NCBI GenBank Accession NP_005505.2 (see also GenBank Accession AUV50118.1.). FIG. 5C provides the sequence HLA-C*0701 (GenBank Accession NP_001229971.1) (HLA-C*07:01:01:01 or HLA-Cw*070101, HLA-Cw*07 see GenBank Accession CAO78194.1).

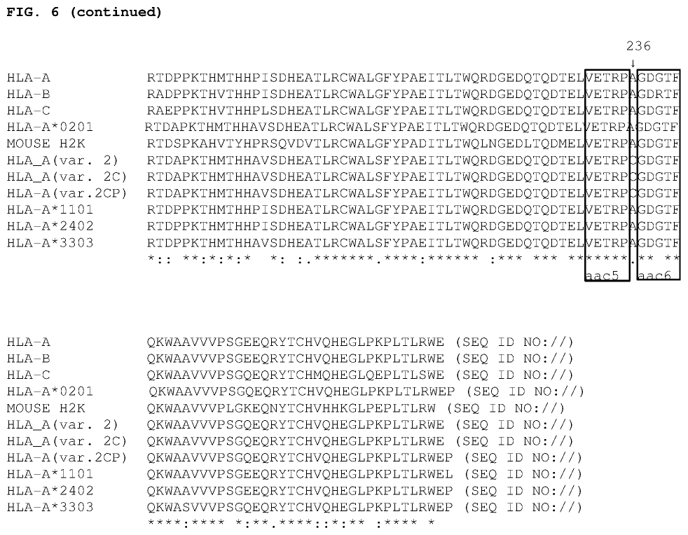

FIG. 6 provides an alignment of eleven mature MHC class I heavy chain amino acid sequences without their leader sequences or transmembrane domains or intracellular domains. The aligned sequences are human HLA-A, HLA-B, and HLA-C, a mouse H2K protein sequence, three variants of HLA-A (var. 1, var. 2C, and var. 2CP), and 3 human HLA-A variants (HLA-A*1101; HLA-A*2402; and HLA-A*3303). Indicated in the alignment are the locations (84 and 139 of the mature proteins) where cysteine residues may be introduced (e.g., by substitution) for the formation of a disulfide bond to stabilize the MHC H chain—β2M complex. Also shown in the alignment is position 236 (of the mature polypeptide), which may be substituted by a cysteine residue that can form an inter-chain disulfide bond with β2M (e.g., at aa 12). An arrow appears above each of those locations and the residues are bolded. The seventh HLA-A sequence shown in the alignment (var. 2c), shows the sequence of variant 2 substituted with C residues at positions 84, 139 and 236. The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

With regard to FIG. 6, in some cases: i) aac1 (amino acid cluster 1) may be the amino acid sequence GTLRG (SEQ ID NO:98) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO:100) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO:101) or that sequence with one or two amino acids deleted or substituted with other naturally occurring amino acids (e.g., A replaced by G, Q replaced by N, or T replaced by S, and/or K replaced by R or Q); v) aac5 (amino acid cluster 5) may be the amino acid sequence VETRP (SEQ ID NO:102) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO:103) or that sequence with one or two amino acids deleted or substituted with other naturally occurring amino acids (e.g., D replaced by E, T replaced by S, or F replaced by L, W, or Y).

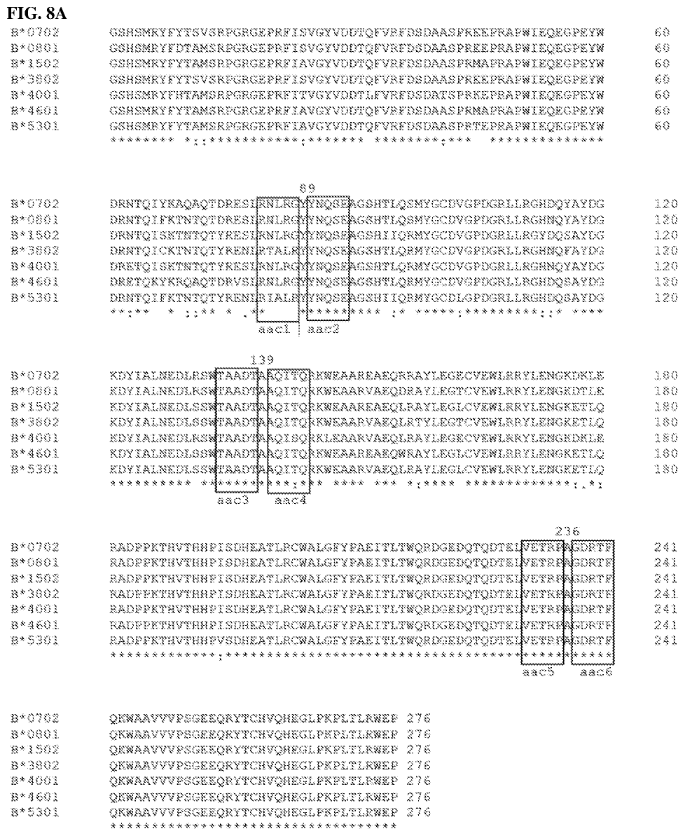

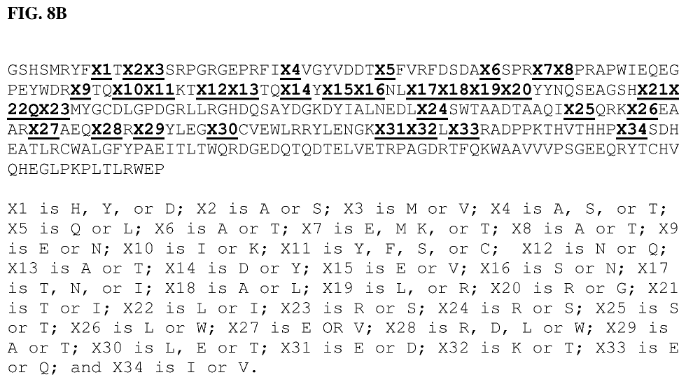

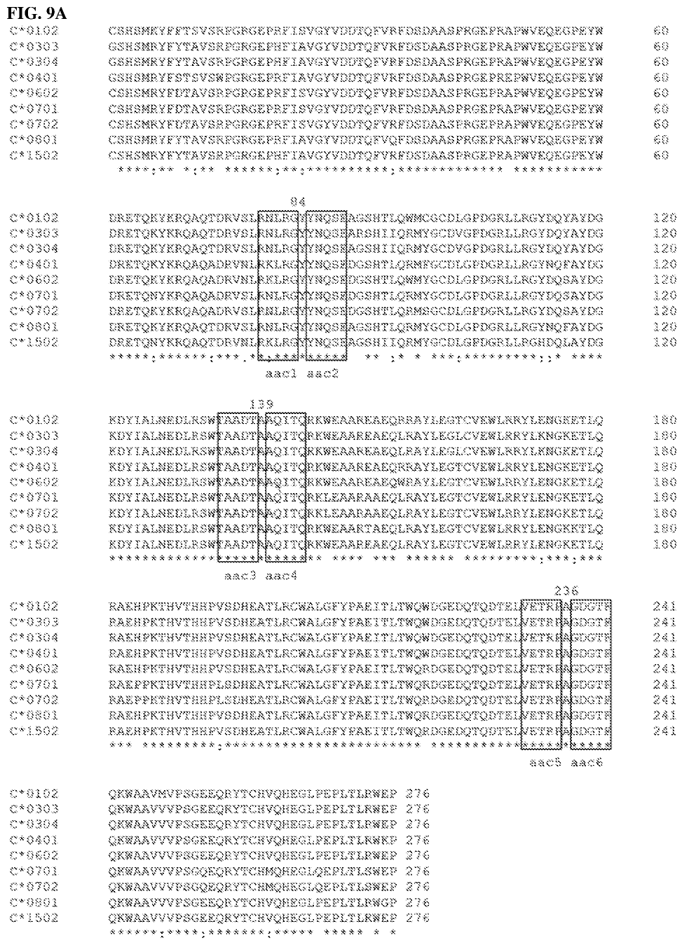

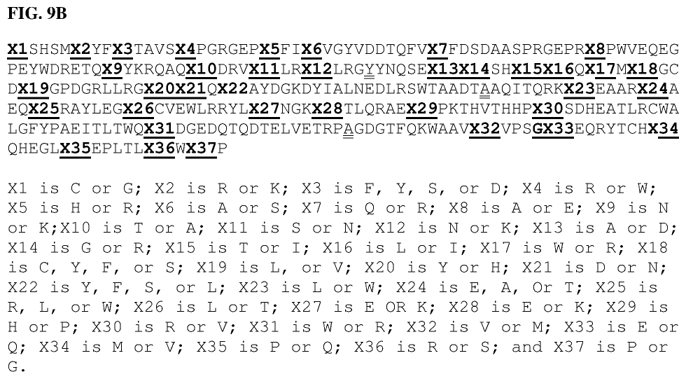

FIGS. 7-9 provide alignments of mature HLA class I heavy chain amino acid sequences (without leader sequences or transmembrane domains or intracellular domains). The aligned amino acid sequences in FIG. 7A are HLA-A class I heavy chains of the following alleles: A*0101, A*0201, A*0301, A*1101, A*2301, A*2402, A*2407, A*3303, and A*3401. The aligned amino acid sequences in FIG. 8A are HLA-B class I heavy chains of the following alleles: B*0702, B*0801, B*1502, B*3802, B*4001, B*4601, and B*5301. The aligned amino acid sequences in FIG. 9A are HLA-C class I heavy chains of the following alleles: C*0102, C*0303, C*0304, C*0401, C*0602, C*0701, C*0801, and C*1502. Indicated in the alignments are the locations (84 and 139 of the mature proteins) where cysteine residues may be introduced (e.g., by substitution) for the formation of a disulfide bond to stabilize the HLA H chain—β2M complex. Also shown in the alignment is position 236 (of the mature polypeptide), which may be substituted by a cysteine residue that can form an inter-chain disulfide bond with β2M (e.g., at aa 12). The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

FIGS. 7A, 8A, and 9A provide alignments of the amino acid sequences of mature HLA-A, -B, and -C class I heavy chains, respectively. The sequences are provided for the extracellular portion of the mature protein (without leader sequences or transmembrane domains or intracellular domains). As described in FIG. 6, the positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine ae also shown. FIGS. 7B, 8B, and 9B provide consensus amino acid sequences for the HLA-A, -B, and -C sequences, respectively, provide in FIGS. 7A, 8A, and 9A. The consensus sequences show the variable amino acid positions as “X” residues sequentially numbered and the locations of amino acids 84, 139 and 236 double underlined.

With regard to FIG. 7A, in some cases: i) aac1 (amino acid cluster 1) may be the amino acid sequence GTLRG (SEQ ID NO: 98) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO: 99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO: 100) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO: 101) or that sequence with one or two amino acids deleted or substituted with other naturally occurring amino acids (e.g., A replaced by G, Q replaced by N, or T replaced by S, and or K replaced by R or Q); v) aac5 (amino acid cluster 5) may be the amino acid sequence VETRP (SEQ ID NO: 102) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO: 103) or that sequence with one or two amino acids deleted or substituted with other naturally occurring amino acids (e.g., D replaced by E, T replaced by S, or F replaced by L, W, or Y).

With regard to FIG. 8A, in some cases: i) aac1 (amino acid cluster 1) may be the amino acid sequence RNLRG (SEQ ID NO: 104) or that sequence with one or two amino acids deleted or substituted with other naturally occurring amino acids (e.g., N replaced by T or I; and/or L replaced by A; and/or the second R replaced by L; and/or the G replaced by R); ii) aac2 (amino acid cluster 2) may be the amino acid sequence YNQSE (SEQ ID NO: 99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:105) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:106) or that sequence with one or two amino acids deleted or substituted with other naturally occurring amino acids (e.g., A replaced by G; and/or the first Q replaced by N; and/or I replaced by L or V; and/or the T replaced by S; and/or the second Q replaced by N); v) aac5 (amino acid cluster 5) may be the amino acid sequence VETRP (SEQ ID NO: 102) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDRTF (SEQ ID NO:107) or that sequence with one or two amino acids deleted or substituted with other naturally occurring amino acids (e.g., D replaced by E; and/or T replaced by S; and/or R replaced by K or H; and/or F replaced by L, W, or Y).

With regard to FIG. 9A, in some cases: i) aac1 (amino acid cluster 1) may be the amino acid sequence RNLRG (SEQ ID NO:104) or that sequence with one or two amino acids deleted or substituted with other naturally occurring amino acids (e.g., N replaced by K; and/or L replaced by A or I; and/or the second R replaced by H; and/or the G replaced by T or S); ii) aac2 (amino acid cluster 2) may be the amino acid sequence YNQSE (SEQ ID NO:99) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:105) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:106) or that sequence with one or two amino acids deleted or substituted with other naturally occurring amino acids (e.g., A replaced by G; and/or the first Q replaced by N; and/or I replaced by L; and/or the second Q replaced by N or K); v) aac5 (amino acid cluster 5) may be the amino acid sequence VETRP (SEQ ID NO:102) or that sequence with one or two amino acids deleted or substituted with other naturally occurring amino acids (e.g., V replaced by I or L, E replaced by D, T replaced by S, and/or R replaced by K or H); and/or vi) aac6 (amino acid cluster 6) may be the amino acid sequence GDGTF (SEQ ID NO:103) or that sequence with one or two amino acids deleted or substituted with other naturally occurring amino acids (e.g., D replaced by E; and/or T replaced by S; and/or F replaced by L, W, or Y).

### HLA-A

In some cases, a TMMP of the present disclosure comprises an HLA-A heavy chain polypeptide. The HLA-A heavy chain peptide sequences, or portions thereof, that may be that may be incorporated into a TMMP of the present disclosure include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 7A. Any of those alleles may comprise a mutation at one or more of positions 84, 139 and/or 236 (as shown in FIG. 7A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, HLA-A sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-A alleles may also be employed (e.g., it may comprise 1-25, 1-5, 5-10, 10-15, 15-20, 20-25, or 25-30 amino acid insertions, deletions, and/or substitutions).

In some cases, a TMMP of the present disclosure comprises an HLA-A heavy chain polypeptide comprising the following HLA-A consensus amino acid sequence:

(SEQ ID NO: 61)
GSHSMRYFX1TSVSRPGRGEPRFIAVGYVDDTQFVRFDSDAASQX2MEPR
APWIEQEGPEYWDX3X4TX5X6X7KAX8SQX9X10RX11X12LX13X14
X15X16X17YYNQSEX18GSHTX19QX20MX21GCDVGX22DX23RFLRG
YX24QX25AYDGKDYIALX26EDLRSWTAADMAAQX27TX287X29KWE
X30X31X32EAEQX33RX34YLX35GX36CVX37X38LRRYLENGKETLQ
RTDX39PKTHMTHHX40X41SDHEATLRCWALX42FYPAEITLTWQRDGE
DQTQDTELVETRPAGDGTFQKWAX43VVVPSGX44EQRYTCHVQHEGLPK
PLTLRWEX45,

wherein X1 is F, Y, S, or T; X2 is K or R; X3 is Q, G, E, or R; X4 is N or E; X5 is R or G; X6 is N or K; X7 is M or V; X8 is H or Q; X9 is T or I; X10 is D or H; X11 is A, V, or E; X12 is N or D; X13 is G or R; X14 is T or 1; X15 is L or A; X16 is R or L; X17 is G or R; X18 is A or D; X19 is 1, L, or V; X20 is 1, R or M; X21 is F or Y; X22 is S or P; X23 is W or G; X24 is R, H, or Q; X25 is D or Y; X26 is Nor K; X27 is T or T; X28 is K or Q; X29 is R or H; X30 is A or T; X31 is A or V; X32 is H or R; X33 is R, L, Q, or W; X34 is V or A; X35 is D or E; X36 is R or T; X37 is D or E; X38 is W or G; X39 is P or A; X40 is P or A; X41 is V or I; X42 is S or G; X43 is A or S; X44 is Q or E; and X45 is P or L.

As one example, an MHC Class I heavy chain polypeptide of a TMMP can comprise an amino acid sequence having at least 75%, at least 80%, at least 85%, at least 90%, at least 95%, at least 98%, at least 99%, or 100%, amino acid sequence identity to the following human HLA-A heavy chain amino acid sequence:

(SEQ ID NO: 108)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP.

In some cases, an HLA-A heavy chain polypeptide suitable for inclusion in a TMMP of the present disclosure comprises the following amino acid sequence: GSHSMRYFFTSVSRPGRGEPRFIAVGYVDDTQFVRFDSDAASQRMEPRAPWIEQEGPEYWDGET RKVKAHSQTHRVDLGTLRGYYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPAGDGTFQKWAAVV VPSGQEQRYTCHVQHEGLPKPLTLRWEP (SEQ ID NO:109). This HLA-A heavy chain polypeptide is also referred to as “HLA-A*0201” or simply “HLA-A02.” In some cases, the C-terminal Pro is not included in a TMMP of the present disclosure. For example, in some cases, an HLA-A02 polypeptide suitable for inclusion in a TMMP of the present disclosure comprises the following amino acid sequence.

(SEQ ID NO: 110)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWE.

### HLA-A (Y84A; A236C)

In some cases, the MHC Class I heavy chain polypeptide comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A heavy chain (Y84A; A236C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGAYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWEP (SEQ ID NO:111), where amino acid 84 is Ala and amino acid 236 is Cys. In some cases, the Cys-236 forms an interchain disulfide bond with Cys-12 of a variant β2M polypeptide that comprises an R12C substitution.

In some cases, an HLA-A heavy chain polypeptide suitable for inclusion in a TMMP of the present disclosure is an HLA-A02 (Y84A; A236C) polypeptide comprising the following amino acid sequence:

(SEQ ID NO: 112)
GSHSMRYFFTSVSRPGRGEPRFIAVGYVDDTQFVRFDSDAASQRMEPRAP
WIEQEGPEYWDGETRKVKAHSQTHRVDLGTLRGAYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWEP.

In some cases, an HLA-A heavy chain polypeptide suitable for inclusion in a TMMP of the present disclosure is an HLA-A02 (Y84A; A236C) polypeptide comprising the following amino acid sequence:

(SEQ ID NO: 113)
GSHSMRYFFTSVSRPGRGEPRFIAVGYVDDTQFVRFDSDAASQRMEPRAP
WIEQEGPEYWDGETRKVKAHSQTHRVDLGTLRGAYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWE.

### HLA-A (Y84C; A139C)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A heavy chain (Y84C; A139C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMCAQTTKHKWEAAHVAEQLRAYLEGTCVEWLRRYLENGKETLQRTDAPK THMTHHAVSDHEATLRCWALSFYPAEITLTWQRDGEDQTQDTELVETRPAGDGTFQKWAAVV VPSGQEQRYTCHVQHEGLPKPLTLRWEP (SEQ ID NO:114), where amino acid 84 is Cys and amino acid 139 is Cys. In some cases, Cys-84 forms an intrachain disulfide bond with Cys-139.

### HLA-A11 (HLA-A*1101)

As one non-limiting example, an MHC Class I heavy chain polypeptide of a TMMP can comprise an amino acid sequence having at least 75%, at least 80%, at least 85%, at least 90%, at least 95%, at least 98%, at least 99%, or 100%, amino acid sequence identity to the following human HLA-A11 heavy chain amino acid sequence: GSHSMRYFYTSVSRPGRGEPRFIAVGYVDDTQFVRFDSDAASQRMEPRAPWIEQEGPEYWDQE TRNVKAQSQTDRVDLGTLRGYYNQSEDGSHTIQIMYGCDVGPDGRFLRGYRQDAYDGKDYIA LNEDLRSWTAADMAAQITKRKWEAAHAAEQQRAYLEGTCVEWLRRYLENGKETLQRTDPPKT HMTHHPISDHEATLRCWALGFYPAEITLTWQRDGEDQTQDTELVETRPAGDGTFQKWAAVVV PSGEEQRYTCHVQHEGLPKPLTLRWE (SEQ ID NO:115). Such an MHC Class I heavy chain may be prominent in Asian populations, including populations of individuals of Asian descent.

### HLA-A11 (Y84A; A236C)

As one non-limiting example, in some cases, the MHC Class I heavy chain polypeptide is an HLA-A11 allele that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A A11 heavy chain (Y84A; A236C) amino acid sequence: GSHSMRYFYTSVSRPGRGEPRFIAVGYVDDTQFVRFDSDAASQRMEPRAPWIEQEGPEYWDQE TRNVKAQSQTDRVDLGTLRGAYNQSEDGSHTIQIMYGCDVGPDGRFLRGYRQDAYDGKDYIA LNEDLRSWTAADMAAQITKRKWEAAHAAEQQRAYLEGTCVEWLRRYLENGKETLQRTDPPKT HMTHHPISDHEATLRCWALGFYPAEITLTWQRDGEDQTQDTELVETRPCGDGTFQKWAAVVV PSGEEQRYTCHVQHEGLPKPLTLRWE (SEQ ID NO:116), where amino acid 84 is Ala and amino acid 236 is Cys. In some cases, the Cys-236 forms an interchain disulfide bond with Cys-12 of a variant β2M polypeptide that comprises an R12C substitution.

### HLA-A24 (HLA-A*2402)

As one non-limiting example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-A24 heavy chain amino acid sequence: GSHSMRYFSTSVSRPGRGEPRFIAVGYVDDTQFVRFDSDAASQRMEPRAPWIEQEGPEYWDEET GKVKAHSQTDRENLRIALRYYNQSEAGSHTLQMMFGCDVGSDGRFLRGYHQYAYDGKDYIAL KEDLRSWTAADMAAQITKRKWEAAHVAEQQRAYLEGTCVDGLRRYLENGKETLQRTDPPKT HMTHHPISDHEATLRCWALGFYPAEITLTWQRDGEDQTQDTELVETRPAGDGTFQKWAAVVV PSGEEQRYTCHVQHEGLPKPLTLRWEPSSQPTVPIVGIIAGLVLLGAVITGAVVAAVMWRRNSS DRKGGSYSQAASSDSAQGSDVSLTACKV (SEQ ID NO:117). Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is an Cys and amino acid 236 is a Cys.

### HLA-A33 (HLA-A*3303)

As one non-limiting example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-A33 heavy chain amino acid sequence: GSHSMRYFTTSVSRPGRGEPRFIAVGYVDDTQFVRFDSDAASQRMEPRAPWIEQEGPEYWDRN TRNVKAHSQIDRVDLGTLRGYYNQSEAGSHTIQMMYGCDVGSDGRFLRGYQQDAYDGKDYIA LNEDLRSWTAADMAAQITQRKWEAARVAEQLRAYLEGTCVEWLRRYLENGKETLQRTDPPKT HMTHHAVSDHEATLRCWALSFYPAEITLTWQRDGEDQTQDTELVETRPAGDGTFQKWASVVV PSGQEQRYTCHVQHEGLPKPLTLRWEPSSQPTIPIVGIIAGLVLFGAVFAGAVVAAVRWRRKSSD RKGGSYSQAASSDSAQGSDMSLTACKV (SEQ ID NO:118). Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is an Cys and amino acid 236 is a Cys.

### HLA-B

In some cases, a TMMP of the present disclosure comprises an HLA-B heavy chain polypeptide. The HLA-B heavy chain peptide sequences, or portions thereof, that may be that may be incorporated into a TMMP of the present disclosure include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 8A. Any of those alleles may comprise a mutation at one or more of positions 84, 139 and/or 236 (as shown in FIG. 8A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, a HLA-B polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-B alleles may also be employed (e.g., it may comprise 1-25, 1-5, 5-10, 10-15, 15-20, 20-25, or 25-30 amino acid insertions, deletions, and/or substitutions).

In some cases, a TMMP of the present disclosure comprises an HLA-B heavy chain polypeptide comprising the following HLA-B consensus amino acid sequence:

(SEQ ID NO: 69)
GSHSMRYFX1TX2X3SRPGRGEPRFIX4VGYVDDTX5FVRFDSDAX6SPR
X7X8PRAPWIEQEGPEYWDRX9TQX10X11KTX12X13TQX14YX15X16
NLX17X18X19X20YYNQSEAGSHX21X22QX23MYGCDLGPDGRLLRGH
DQSAYDGKDYIALNEDLX24SWTAADTAAQIX25QRKX26EAARX27AEQ
X28RX29YLEGX30CVEWLRRYLENGKX31X32LX33RADPPKTHVTHHP
X34SDHEATLRCWALGFYPAEITLTWQRDGEDQTQDTELVETRPAGDRTF
QKWAAVVVPSGEEQRYTCHVQHEGLPKPLTLRWEP,

wherein X1 is H, Y, or D; X2 is A or S; X3 is M or V; X4 is A, S, or T; X5 is Q or L; X6 is A or T; X7 is E, M K, or T; X8 is A or T; X9 is E or N; X10 is I or K; X11 is Y, F, S, or C; X12 is N or Q; X13 is A or T; X14 is D or Y; X15 is E or V; X16 is S or N; X17 is T, N, or I; X18 is A or L; X19 is L, or R; X20 is R or G; X21 is T or I; X22 is L or I; X23 is R or S; X24 is R or S; X25 is S or T; X26 is L or W; X27 is E OR V; X28 is R, D, L or W; X29 is A or T; X30 is L, E or T; X31 is E or D; X32 is K or T; X33 is E or Q; and X34 is I or V.

As an example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-B heavy chain amino acid sequence:

(SEQ ID NO: 119)
GSHSMRYFYTSVSRPGRGEPRFISVGYVDDTQFVRFDSDAASPREEPRAP
WIEQEGPEYWDRNTQIYKAQAQTDRESLRNLRGYYNQSEAGSHTLQSMYG
CDVGPDGRLLRGHDQYAYDGKDYIALNEDLRSWTAADTAAQITQRKWEAA
REAEQRRAYLEGECVEWLRRYLENGKDKLERADPPKTHVTHHPISDHEAT
LRCWALGFYPAEITLTWQRDGEDQTQDTELVETRPAGDRTFQKWAAVVVP
SGEEQRYTCHVQHEGLPKPLTLRWEP.

### HLA-B (Y84A; A236C)

As one non-limiting example, in some cases, the MHC Class I heavy chain polypeptide is an HLA-B polypeptide that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-B heavy chain (Y84A; A236C) amino acid sequence: GSHSMRYFYTSVSRPGRGEPRFISVGYVDDTQFVRFDSDAASPREEPRAPWIEQEGPEYWDRNT QIYKAQAQTDRESLRNLRGAYNQSEAGSHTLQSMYGCDVGPDGRLLRGHDQYAYDGKDYIAL NEDLRSWTAADTAAQITQRKWEAAREAEQRRAYLEGECVEWLRRYLENGKDKLERADPPKTH VTHHPISDHEATLRCWALGFYPAEITLTWQRDGEDQTQDTELVETRPCGDRTFQKWAAVVVPS GEEQRYTCHVQHEGLPKPLTLRWEP (SEQ ID NO:121), where amino acid 84 is Ala and amino acid 236 is Cys. In some cases, the Cys-236 forms an interchain disulfide bond with Cys-12 of a variant β2M polypeptide that comprises an R12C substitution.

### HLA-B (Y84C; A139C)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-B heavy chain (Y84C; A139C) amino acid sequence: GSHSMRYFYTSVSRPGRGEPRFISVGYVDDTQFVRFDSDAASPREEPRAPWIEQEGPEYWDRNT QIYKAQAQTDRESLRNLRGCYNQSEAGSHTLQSMYGCDVGPDGRLLRGHDQYAYDGKDYIAL NEDLRSWTAADTCAQITQRKWEAAREAEQRRAYLEGECVEWLRRYLENGKDKLERADPPKTH VTHHPISDHEATLRCWALGFYPAEITLTWQRDGEDQTQDTELVETRPAGDRTFQKWAAVVVPS GEEQRYTCHVQHEGLPKPLTLRWEP (SEQ ID NO:122), where amino acid 84 is Cys and amino acid 139 is Cys. In some cases, Cys-84 forms an intrachain disulfide bond with Cys-139.

### HLA-B*0702

As an example, in some cases, a MHC Class I heavy chain polypeptide present in a TMMP of the present disclosure comprises an amino acid sequence of HLA-B*0702 (SEQ ID NO:62) in FIG. 8A, or a sequence having at least 75% (e.g., at least 80%, at least 85%, at least 90%, at least 95%, at least 98%, at least 99%) or 100%, amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-B heavy chain polypeptide of TMMP of the present disclosure has less than 100% identity to the sequence labeled HLA-B in FIG. 6, or labeled “B*0702 in FIG. 8A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-B heavy chain polypeptide of TMMP of the present disclosure comprises Y84A and A236C substitutions. In some cases, the HLA-B*0702 heavy chain polypeptide of TMMP of the present disclosure comprises Y84C and A139C substitutions. In some cases, the HLA-B heavy chain polypeptide of TMMP of the present disclosure comprises Y84C, A139C, and A236C substitutions.

### HLA-C

In some cases, a TMMP of the present disclosure comprises an HLA-C heavy chain polypeptide. The HLA-C heavy chain polypeptide, or portions thereof, that may be that may be incorporated into a TMMP of the present disclosure include, but are not limited to, the alleles: C*0102, C*0303, C*0304, C*0401, C*0602, C*0701, C*0801, and C*1502, which are aligned without all, or substantially all, of the leader, transmembrane and cytoplasmic sequences in FIG. 9A. Any of those alleles may comprise a mutation at one or more of positions 84, 139 and/or 236 (as shown in FIG. 9A) selected from: a tyrosine to alanine substitution at position 84 (Y84A); a tyrosine to cysteine substitution at position 84 (Y84C); an alanine to cysteine substitution at position 139 (A139C); and an alanine to cysteine substitution at position 236 (A236C). In addition, an HLA-C polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-C alleles may also be employed (e.g., it may comprise 1-25, 1-5, 5-10, 10-15, 15-20, 20-25, or 25-30 amino acid insertions, deletions, and/or substitutions).

In some cases, a TMMP of the present disclosure comprises an HLA-C heavy chain polypeptide comprising the following HLA-C consensus amino acid sequence:

X1SHSMX2YFX3TAVSX4PGRGEPX5FIX6VGYVDDTQFVX7FDSDAASPRGEPRX8 PWVEQEGPEYWDRETQX9YKRQAQX10DRVX11LRX12LRGYYNQSEX13X14SHX15X16QX17MX18GCDX19GPDGRLLRGX20X21QX22AYDGKDYIALNEDLRSWTAADTAAQITQRKX23EA ARX24AEQX25RAYLEGX26CVEWLRRYLX27NGKX28TLQRAEX29PKTHVTHHPX30SDHEAT LRCWALGFYPAEITLTWQX31DGEDQTQDTELVETRPAGDGTFQKWAAVX32VPSGX33EQRYT CHX34QHEGLX35EPLTLX36WX37P (SEQ ID NO:79), wherein X1 is C or G; X2 is R or K; X3 is F, Y, S, or D; X4 is R or W; X5 is H or R; X6 is A or S; X7 is Q or R; X8 is A or E; X9 is N or K; X10 is T or A; X11 is S or N; X12 is N or K; X13 is A or D; X14 is G or R; X15 is T or I; X16 is L or I; X17 is W or R; X18 is C, Y, F, or S; X19 is L, or V; X20 is Y or H; X21 is D or N; X22 is Y, F, S, or L; X23 is L or W; X24 is E, A, Or T; X25 is R, L, or W; X26 is L or T; X27 is E OR K; X28 is E or K; X29 is H or P; X30 is R or V; X31 is W or R; X32 is V or M; X33 is E or Q; X34 is M or V; X35 is P or Q; X36 is R or S; and X37 is P or G.

As an example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-C heavy chain amino acid sequence:

(SEQ ID NO: 123)
CSHSMRYFDTAVSRPGRGEPRFISVGYVDDTQFVRFDSDAASPRGEPRAP
WVEQEGPEYWDRETQNYKRQAQADRVSLRNLRGYYNQSEDGSHTLQRMYG
CDLGPDGRLLRGYDQSAYDGKDYIALNEDLRSWTAADTAAQITQRKLEAA
RAAEQLRAYLEGTCVEWLRRYLENGKETLQRAEPPKTHVTHHPLSDHEAT
LRCWALGFYPAEITLTWQRDGEDQTQDTELVETRPAGDGTFQKWAAVVVP
SGQEQRYTCHMQHEGLQEPLTLSWEP.

### HLA-C(Y84A; A236C)

As one non-limiting example, in some cases, the MHC Class I heavy chain polypeptide is an HLA-C polypeptide that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-C heavy chain (Y84A; A236C) amino acid sequence: CSHSMRYFDTAVSRPGRGEPRFISVGYVDDTQFVRFDSDAASPRGEPRAPWVEQEGPEYWDRE TQNYKRQAQADRVSLRNLRGAYNQSEDGSHTLQRMYGCDLGPDGRLLRGYDQSAYDGKDYI ALNEDLRSWTAADTAAQITQRKLEAARAAEQLRAYLEGTCVEWLRRYLENGKETLQRAEPPKT HVTHHPLSDHEATLRCWALGFYPAEITLTWQRDGEDQTQDTELVETRPCGDGTFQKWAAVVV PSGQEQRYTCHMQHEGLQEPLTLSWEP (SEQ ID NO:124), where amino acid 84 is Ala and amino acid 236 is Cys. In some cases, the Cys-236 forms an interchain disulfide bond with Cys-12 of a variant β2M polypeptide that comprises an R12C substitution.

### HLA-C(Y84C; A139C)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-C heavy chain (Y84C; A139C) amino acid sequence: CSHSMRYFDTAVSRPGRGEPRFISVGYVDDTQFVRFDSDAASPRGEPRAPWVEQEGPEYWDRE TQNYKRQAQADRVSLRNLRGCYNQSEDGSHTLQRMYGCDLGPDGRLLRGYDQSAYDGKDYI ALNEDLRSWTAADTCAQITQRKLEAARAAEQLRAYLEGTCVEWLRRYLENGKETLQRAEPPKT HVTHHPLSDHEATLRCWALGFYPAEITLTWQRDGEDQTQDTELVETRPAGDGTFQKWAAVVV PSGQEQRYTCHMQHEGLQEPLTLSWEP (SEQ ID NO:125), where amino acid 84 is Cys and amino acid 139 is Cys. In some cases, Cys-84 forms an intrachain disulfide bond with Cys-139.

### HLA-C*0701

In some cases, a MHC Class I heavy chain polypeptide of a TMMP of the present disclosure comprises an amino acid sequence of HLA-C*0701 of FIG. 9A (labeled HLA-C in FIG. 6), or an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-C heavy chain polypeptide of a TMMP of the present disclosure has less than 100% identity to the sequence labeled HLA-C*0701 in FIG. 9A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-C heavy chain polypeptide of a TMMP of the present disclosure comprises Y84A and A236C substitutions. In some cases, the HLA-C*0701 heavy chain polypeptide of a T-Cell-MMP or its epitope conjugate comprises Y84C and A139C substitutions. In some cases, the HLA-C heavy chain polypeptide of a TMMP of the present disclosure comprises Y84C, A139C, and A236C substitutions.

### Non-Classical HLA-E, -F, and -G MHC Class I Heavy Chains

In some cases, a TMMP of the present disclosure comprises a non-classical MHC Class I heavy chain polypeptide. Among the non-classical HLA heavy chain polypeptides, or portions thereof, that may be that may be incorporated into a TMMP of the present disclosure include, but are not limited to, those of HLA-E, -F, and -G alleles. Amino acid sequences for HLA-E, -F, and -G heavy chain polypeptides, (and the HLA-A, B and C alleles) may be found on the world wide web hla.alleles.org/nomenclature/index.html, the European Bioinformatics Institute (www(dot)ebi(dot)ac(dot)uk), which is part of the European Molecular Biology Laboratory (EMBL), and at the National Center for Biotechnology Information (www(dot)ncbi(dot)nlm(dot)nih(dot)gov).

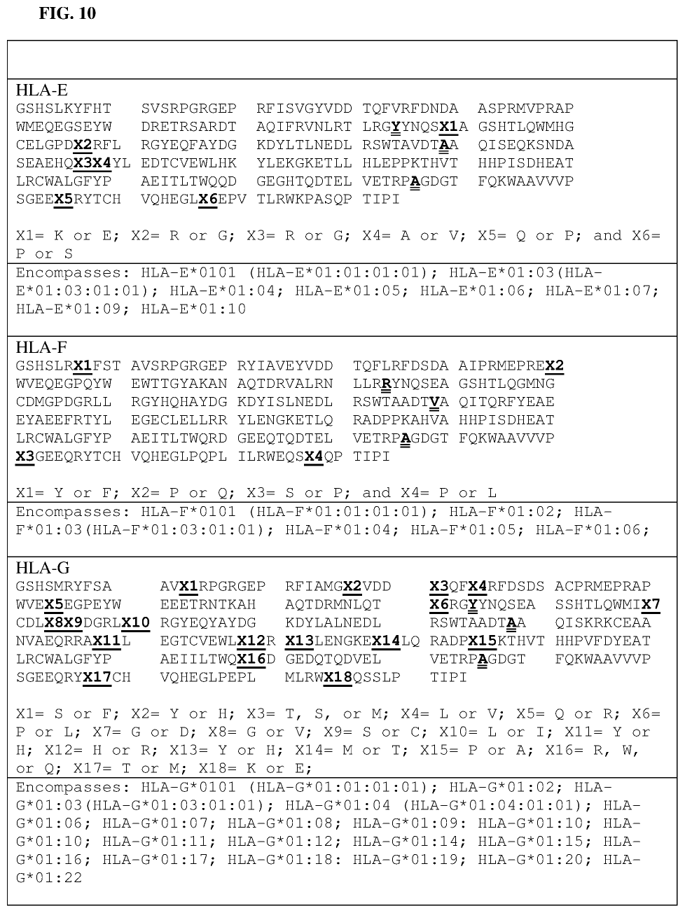

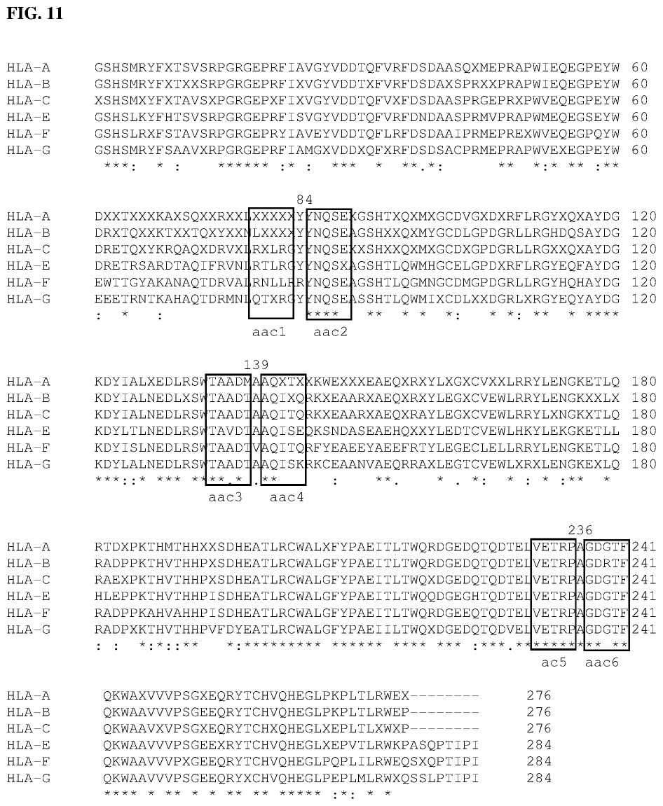

Non-limiting examples of suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Non-limiting examples of suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA E, -F and -G alleles without all, or substantially all, of the leader, transmembrane and cytoplasmic sequences are provided in FIG. 10, and aligned with consensus sequences of the above-mentioned HLA-A, -B and -C alleles in FIG. 11.

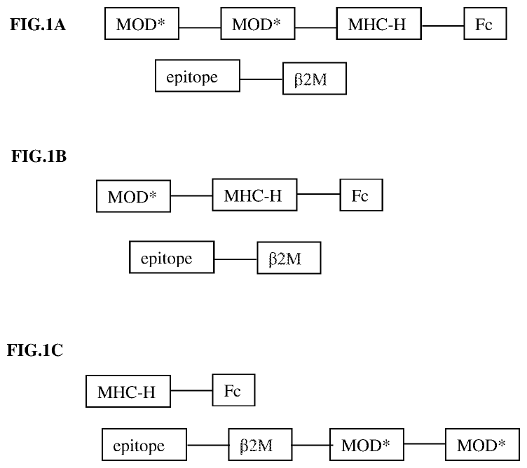

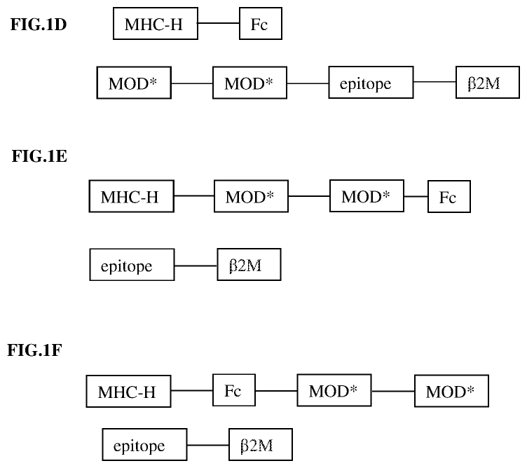

FIG. 1—provides a consensus sequence for each of HLA-E, -F, and -G with the variable aa positions indicated as “X” residues sequentially numbered and the locations of aas 84, 139 and 236 double underlined.

FIG. 11 provides an alignment of the consensus amino acid sequences for HLA-A, -B, -C, -E, -F, and -G, which are given in FIGS. 7-11. Variable residues in each sequence are listed as “X” with the sequential numbering removed. As indicated in FIG. 6, the locations of aas 84, 139 and 236 are indicated with their flanking five-amino acid clusters that may be replaced by 1 to 5 amino acids selected independently from (i) any naturally occurring amino acid or (ii) any naturally occurring amino acid except proline or glycine are also shown.

Any of the above-mentioned HLA-E, -F, and/or -G alleles may comprise a substitution at one or more of positions 84, 139 and/or 236 as shown in FIG. 11 for the consensus sequences. In some cases, the substitutions may be selected from a: position 84 tyrosine to alanine (Y84A) or cysteine (Y84C), or, in the case of HLA-F, an R84A or R84C substitution; a position 139 alanine to cysteine (A139C), or, in the case of HLA-F, a V139C; and an alanine to cysteine substitution at position 236 (A236C). In addition, an HLA-E, -F and/or -G sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of any of the consensus sequences of set forth in FIG. 11 may also be employed (e.g., the sequences may comprise 1-25, 1-5, 5-10, 10-15, 15-20, 20-25, or 25-30 amino acid insertions, deletions, and/or substitutions in addition to changes at variable residues listed therein).

### Mouse H2K

In some cases, a MHC Class I heavy chain polypeptide present in a TMMP of the present disclosure comprises an amino acid sequence of MOUSE H2K (SEQ ID NO:45) (MOUSE H2K in FIG. 6), or a sequence having at least 75%, at least 80%, at least 85%, at least 90%, at least 95%, at least 98%, at least 99%, or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the MOUSE H2K heavy chain polypeptide of a TMMP of the present disclosure has less than 100% identity to the sequence labeled MOUSE H2K in FIG. 6, it may comprise a mutation at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In some cases, the MOUSE H2K heavy chain polypeptide of a TMMP of the present disclosure comprises Y84A and A236C substitutions. In some cases, the MOUSE H2K heavy chain polypeptide of a TMMP of the present disclosure comprises Y84C and A139C substitutions. In some cases, the MOUSE H2K heavy chain polypeptide of a TMMP of the present disclosure comprises Y84C, A139C and A236C substitutions.

### Exemplary Combinations

Table 1, below, presents various combinations of MHC Class I heavy chain sequence modifications that can be incorporated in a TMMP of the present disclosure.

TABLE 1

| | | | Specific |
|---|---|---|---|
| | | Sequence | Substitutions at aa |
| | HLA Heavy | Identity | positions 84, 139 |
| Entry | Chain Sequence | Range [symbol] | and/or 236 |
| 1 | HLA-A | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 7B) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 2 | A*0101, A*0201, | 75%-99.8%, 80%-99.8%, 85%-99.8%, | None; Y84C; Y84A; |
| | A*0301, A*1101, | 90%-99.8%, 95%-99.8%, 98%-99.8%, | A139C; A236C; |
| | A*2402, A*2301, | or 99%-99.8%; or 1-25, 1-5, 5-10, 10- | (Y84A & A236C); |
| | A*2402, A*2407, | 15, 15-20, or 20-25 aa insertions, | (Y84C & A139C); or |
| | A*3303, or | deletions, and/or substitutions | (Y84C, A139C & |
| | A*3401 | | A236C) |
| | (FIG. 7A) | | |
| 3 | HLA-B | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 8B) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 4 | B*0702, B*0801, | 75%-99.8%, 80%-99.8%, 85%-99.8%, | None; Y84C; Y84A; |
| | B*1502, B*3802, | 90%-99.8%, 95%-99.8%, 98%-99.8%, | A139C; A236C; |
| | B*4001, B*4601, | or 99%-99.8%; or 1-25, 1-5, 5-10, 10- | (Y84A & A236C); |
| | or B*5301 | 15, 15-20, or 20-25 aa insertions, | (Y84C & A139C); or |
| | (FIG. 8A) | deletions, and/or substitutions | (Y84C, A139C & |
| | | | A236C) |
| 5 | HLA-C | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 9B) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 6 | C*0102, C*0303, | 75%-99.8%, 80%-99.8%, 85%-99.8%, | None; Y84C; Y84A; |
| | C*0304, C*0401, | 90%-99.8%, 95%-99.8%, 98%-99.8%, | A139C; A236C; |
| | C*0602, C*0701, | or 99%-99.8%; or 1-25, 1-5, 5-10, 10- | (Y84A & A236C); |
| | C*0801, or | 15, 15-20, or 20-25 aa insertions, | (Y84C & A139C); or |
| | C*1502 | deletions, and/or substitutions | (Y84C, A139C & |
| | (FIG. 9A) | | A236C) |
| 7 | HLA-E, F, or G | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 10) | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions (not | (Y84C, A139C & |
| | | counting variable residues) | A236C) |
| 8 | MOUSE H2K | 75%-99.8%, 80%-99.8%, 85%-99.8%, | None; Y84C; Y84A; |
| | (FIG. 6) | 90%-99.8%, 95%-99.8%, 98%-99.8%, | A139C; A236C; |
| | | or 99%-99.8%; or 1-25, 1-5, 5-10, 10- | (Y84A & A236C); |
| | | 15, 15-20, or 20-25 aa insertions, | (Y84C & A139C); or |
| | | deletions, and/or substitutions | (Y84C, A139C & |
| | | | A236C) |

[symbol]

The Sequence Identity Range is the permissible range in sequence identity of a MHC-H polypeptide sequence incorporated into a TMMP relative to the corresponding portion of the sequences listed in FIG. 6-11 not counting the variable residues in the consensus sequences.

### Beta-2 Microglobulin

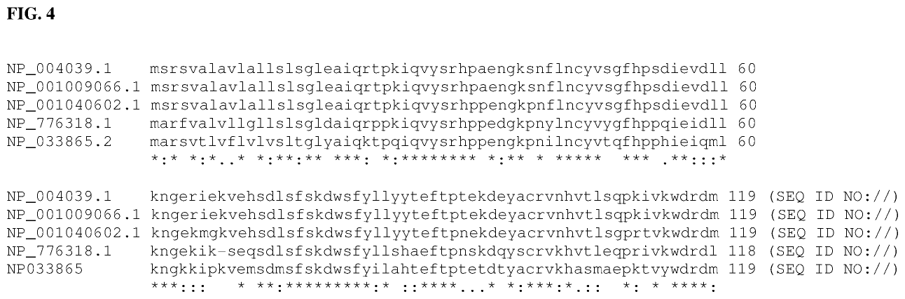

A β2-microglobulin (β2M) polypeptide of a TMMP of the present disclosure can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to a β2M amino acid sequence depicted in FIG. 6. In some instances, a β2M polypeptide comprises an amino acid sequence having at least 75%, at least 80%, at least 85%, at least 90%, at least 95%, at least 98%, at least 99%, or 100%, amino acid sequence identity to amino acids 21 to 119 of a β2M amino acid sequence depicted in FIG. 4.

In some cases, a suitable β2M polypeptide comprises the following amino acid sequence:

IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:126); and the HLA Class I heavy chain polypeptide comprises the following amino acid sequence:

GSHSMRYFFTSVSRPGRGEPRFIAVGYVDDTQFVRFDSDAASQRMEPRAPWIEQE GPEYWDGETRKVKAHSQTHRVDL(aa1){C}(aa2)AGSHTVQRMYGCDVGSDWRFLRGYHQYAY DGKDYIALKEDLRSW(aa3){C}(aa4))HKWEAAHVAEQLRAYLEGTCVEWLRRYLENGKETLQR TDAPKTHMTHHAVSDHEATLRCWALSFYPAEITLTWQRDGEDQTQDTEL(aa5)I(aa6)QKWAAV VVPSGQEQRYTCHVQHEGLPKPLTLRWEP (SEQ ID NO:127), where the cysteine residues indicated as {C} form an disulfide bond between the α1 and α2-1 helices and tII residue forms a disulfide bond with the β2M polypeptide cysteine at position 12. In the sequence above, “aa1” is “amino acid cluster 1”; “aa2” is “amino acid cluster 2”; “aa3” is “amino acid cluster 3”; “aa4” is “amino acid cluster 4”; “aa5” is “amino acid cluster 5”; and “aa6” is “amino acid cluster 6”; see, e.g., FIG. 8. Each occurrence of aa1, aa2, aa3, aa4, aa5, and aa6 is and independently selected to be 1-5 amino acid residues, wherein the amino acid residues are i) selected independently from any naturally occurring (e.g., encoded) amino acid or ii) any naturally occurring amino acid except proline or glycine.

In some cases, an MHC polypeptide comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such cysteine residues, when present in an MHC polypeptide of a first polypeptide of a TMMP of the present disclosure, can form a disulfide bond with a cysteine residue present in a second polypeptide chain of a TMMP of the present disclosure.

In some cases, a first MHC polypeptide in a first polypeptide of a TMMP of the present disclosure, and/or the second MHC polypeptide in the second polypeptide of a TMMP of the present disclosure, includes an amino acid substitution to substitute an amino acid with a cysteine, where the substituted cysteine in the first MHC polypeptide forms a disulfide bond with a cysteine in the second MHC polypeptide, where a cysteine in the first MHC polypeptide forms a disulfide bond with the substituted cysteine in the second MHC polypeptide, or where the substituted cysteine in the first MHC polypeptide forms a disulfide bond with the substituted cysteine in the second MHC polypeptide.

For example, in some cases, one of following pairs of residues in an HLA 02-microglobulin and an HLA Class I heavy chain is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in some cases, residue 236 of the mature HLA-A amino acid sequence is substituted with a Cys. In some cases, residue 236 of the mature HLA-B amino acid sequence is substituted with a Cys. In some cases, residue 236 of the mature HLA-C amino acid sequence is substituted with a Cys. In some cases, residue 32 (corresponding to Arg-12 of mature β2M) of an amino acid sequence depicted in FIG. 4 is substituted with a Cys.

In some cases, a β2M polypeptide comprises the amino acid sequence: IQRTPKIQVY SRHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:128). In some cases, a β2M polypeptide comprises the amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:129).

In some cases, an HLA Class I heavy chain polypeptide comprises the amino acid sequence:

(SEQ ID NO: 130)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP.

In some cases, an HLA Class I heavy chain polypeptide comprises the amino acid sequence:

(SEQ ID NO: 131)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWEP.

In some cases, an HLA Class I heavy chain polypeptide comprises the amino acid sequence:

(SEQ ID NO: 132)
GSHSMRYFFTSVSRPGRGEPRFIAVGYVDDTQFVRFDSDAASQRMEPRAP
WIEQEGPEYWDGETRKVKAHSQTHRVDLGTLRGAYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWE.

In some cases, the β2M polypeptide comprises the following amino acid sequence:

IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:133); and the HLA Class I heavy chain polypeptide of a TMMP of the present disclosure comprises the following amino acid sequence:

GSHSMRYFFTSVSRPGRGEPRFIAVGYVDDTQFVRFDSDAASQRMEPRAPWIEQE GPEYWDGETRKVKAHSQTHRVDLGTLRGYYNQSEAGSHTVQRMYGCDVGSDWRFLRGYHQY AYDGKDYIALKEDLRSWTAADMAAQTTKHKWEAAHVAEQLRAYLEGTCVEWLRRYLENGKE TLQRTDAPKTHMTHHAVSDHEATLRCWALSFYPAEITLTWQRDGEDQTQDTELVETRPCGDGT FQKWAAVVVPSGQEQRYTCHVQHEGLPKPLTLRWEP (SEQ ID NO:134), where the Cys residues that are underlined and in bold form a disulfide bond with one another in the TMMP.

In some cases, the β2M polypeptide comprises the amino acid sequence:

(SEQ ID NO: 135)
IQRTPKIQVYSCHPAENGKSNFLNCYVSGFHPSDIEVDLLKNGERIEKVE
HSDLSFSKDWSFYLLYYTEFTPTEKDENACRVNHVTLSQPKIVKWDRDM.

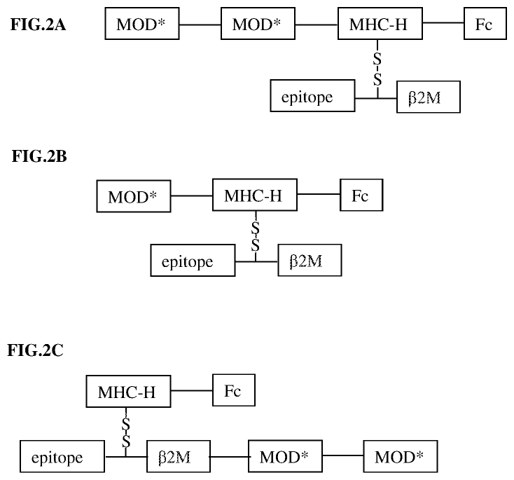

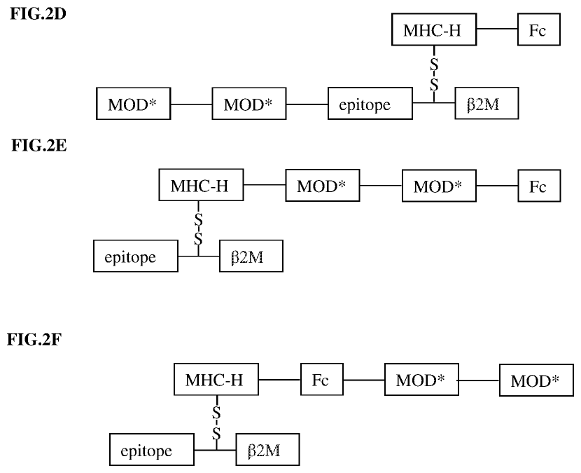

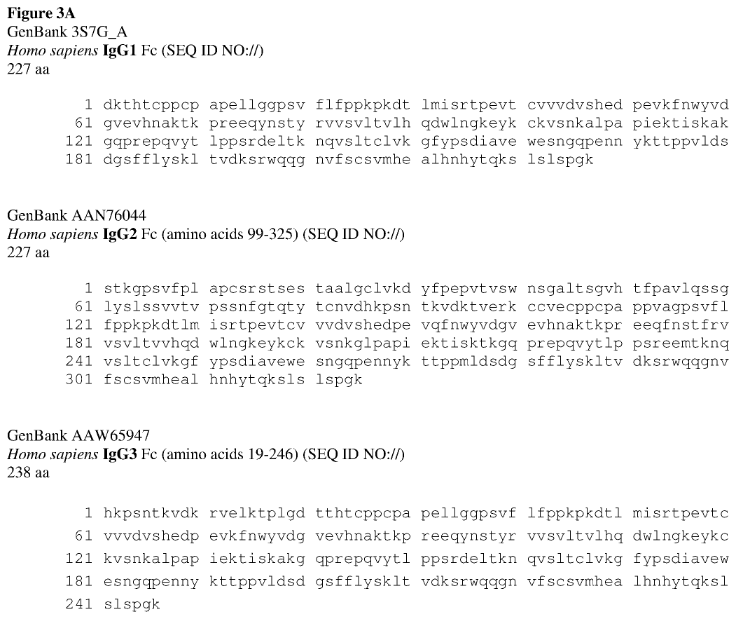

In some cases, the first polypeptide and the second polypeptide of a TMMP of the present disclosure are disulfide linked to one another through: i) a Cys residue present in a linker connecting the peptide epitope and a β2M polypeptide in the first polypeptide chain; and ii) a Cys residue present in an MHC Class I heavy chain in the second polypeptide chain. In some cases, the Cys residue present in the MHC Class I heavy chain is a Cys introduce as a Y84C substitution. In some cases, the linker connecting the peptide epitope and the β2M polypeptide in the first polypeptide chain is GCGGS(G4S)n (SEQ ID NO:136), where n is 1, 2, 3, 4, 5, 6, 7, 8, or 9. For example, in some cases, the linker comprises the amino acid sequence GCGGSGGGGSGGGGSGGGGS (SEQ ID NO:137). As another example, the linker comprises the amino acid sequence GCGGSGGGGSGGGGS (SEQ ID NO:138). Examples of disulfide-linked first and second polypeptides of a TMMP of the present disclosure are depicted schematically in FIG. 2A-2F.

### Multiple Disulfide Bonded TMMPs

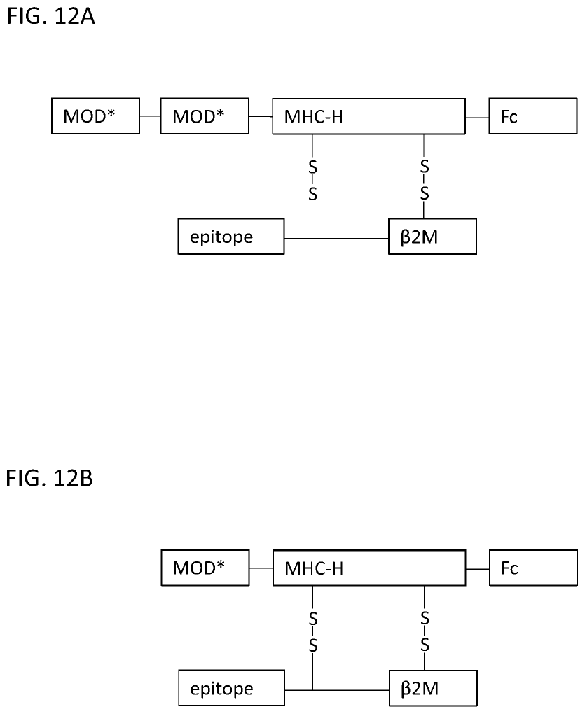

In some cases, the first polypeptide and the second polypeptide of a TMMP of the present disclosure are linked to one another by at least two disulfide bonds (i.e., two interchain disulfide bonds). Examples of such multiple disulfide-linked TMMP are depicted schematically in FIGS. 12A and 12B. In addition, where a TMMP of the present disclosure comprises an IgFc polypeptide, a heterodimeric TMMP can be dimerized, such that disulfide bonds link the IgFc polypeptides in the two heterodimeric TMMPs. Such an arrangement is depicted schematically in FIGS. 12C and 12D, where disulfide bonds are represented by dashed lines. Unless otherwise stated, the at least two disulfide bonds described in the multiple disulfide-linked TMMPPs in this section are not referring to disulfide bonds linking IgFc polypeptides in dimerized TMMPs.

As noted above, in some cases, the first polypeptide and the second polypeptide of a TMMP of the present disclosure are linked to one another by at least two disulfide bonds (i.e., two interchain disulfide bonds). For example, in some instances, the first polypeptide and the second polypeptide of a TMMP of the present disclosure are linked to one another by 2 interchain disulfide bonds. As another example, in some instances, the first polypeptide and the second polypeptide of a TMMP of the present disclosure are linked to one another by 3 interchain disulfide bonds. As another example, in some instances, the first polypeptide and the second polypeptide of a TMMP of the present disclosure are linked to one another by 4 interchain disulfide bonds.

In some cases where a peptide epitope in a first polypeptide of a TMMP of the present disclosure is linked to a β2M polypeptide by a linker comprising a Cys, at least one of the at least two disulfide bonds links a Cys in the linker to a Cys in an MHC Class I heavy chain in the second polypeptide. In some cases, where a peptide epitope in a first polypeptide of a TMMP of the present disclosure is linked to an MHC Class I heavy chain polypeptide by a linker, at least one of the at least two disulfide bonds links a Cys in the linker to a Cys in a β2M polypeptide present in the second polypeptide.

In some cases, a multiple disulfide-linked TMMP of the present disclosure (e.g., a double disulfide-linked TMMP) exhibits increased stability, compared to a control TMMP that includes only one of the at least two disulfide bonds. In some cases, a multiple disulfide-linked TMMP (e.g., a double disulfide-linked TMMP) of the present disclosure exhibits increased in vitro stability, compared to a control TMMP that includes only one of the at least two disulfide bonds. For example, in some cases, a multiple disulfide-linked TMMP of the present disclosure (e.g., a double disulfide-linked TMMP) exhibits at least 5%, at least 10%, at least 15%, at least 20%, at least 25%, at least 50%, at least 2-fold, at least 5-fold, or at least 10-fold, greater in vitro stability, compared to a control TMMP that includes only one of the at least two disulfide bonds.

Whether a multiple disulfide-linked TMMP of the present disclosure exhibits increased in vitro stability, compared to a control TMMP that includes only one of the at least two disulfide bonds, can be determined by measuring the amount disulfide-linked heterodimeric TMMP present in a sample over time and/or under a specified condition and/or during purification of the TMMP.

For example, in some cases, a multiple disulfide-linked TMMP (e.g., a double disulfide-linked TMMP) of the present disclosure exhibits at least 5%, at least 10%, at least 15%, at least 20%, at least 25%, at least 50%, at least 2-fold, at least 5-fold, or at least 10-fold, greater in vitro stability, compared to a control TMMP that includes only one of the at least two disulfide bonds, when the TMMP is stored at 37° C. for a period of time (e.g., for a period of time of from about 1 week to about 2 weeks, from about 2 weeks to about 4 weeks, or from about 4 weeks to about 2 months). For example, in some cases, the amount of disulfide-linked heterodimeric TMMP remaining after storing a multiple disulfide-linked TMMP (e.g., a double disulfide-linked TMMP) of the present disclosure in vitro at 37° C. for 28 days is at least at least 5%, at least 10%, at least 15%, at least 20%, at least 25%, at least 50%, at least 2-fold, at least 5-fold, or at least 10-fold, greater than the amount of disulfide-linked heterodimeric TMMP remaining after storing a control TMMP (a TMMP that includes only one of the at least two disulfide bonds present in the multiple disulfide-linked TMMP) in vitro at 37° C. for 28 days.

In some cases, a multiple disulfide-linked TMMP of the present disclosure exhibits increased in vivo stability, compared to a control TMMP that includes only one of the at least two disulfide bonds. For example, in some cases, a multiple disulfide-linked TMMP of the present disclosure exhibits at least 5%, at least 10%, at least 15%, at least 20%, at least 25%, at least 50%, at least 2-fold, at least 5-fold, or at least 10-fold, greater in vivo stability, compared to a control TMMP that includes only one of the at least two disulfide bonds.

In some cases, the presence of two disulfide bonds in a multiple disulfide-linked TMMP of the present disclosure (e.g., a double disulfide-linked TMMP) provides for increased production of disulfide-linked heterodimeric TMMP, compared to the amount of disulfide-linked heterodimeric TMMP produced when the TMMP is a control TMMP that includes only one of the at least two disulfide bonds. For example, a multiple disulfide-linked TMMP of the present disclosure (e.g., a double disulfide-linked TMMP) can be produced in a mammalian cell in in vitro cell culture, where the mammalian cell is cultured in a liquid cell culture medium. The TMMP can be secreted into the cell culture medium. The cells can be lysed, generating a cell lysate, and the TMMP can be present in the cell lysate. The TMMP can be purified from the cell culture medium and/or the cell lysate. For example, where the TMMP comprises an IgG1 Fc polypeptide, the cell culture medium and/or the cell lysate can be contacted with immobilized protein A (e.g., the cell culture medium and/or the cell lysate can be applied to a protein A column, where protein A is immobilized onto beads). TMMP present in the cell culture medium and/or the cell lysate becomes bound to the immobilized protein A. After washing the column to remove unbound materials, the bound TMMP is eluted, generating a protein A eluate. The amount of disulfide-linked heterodimeric TMMP present in the protein A eluate is a least 0.5%, at least 1%, at least 2%, at least 3%, at least 4%, at least 5%, at least 6%, at least 7%, at least 8%, at least 9%, or at least 10%, higher than the amount of disulfide-linked heterodimeric TMMP present in the protein A eluate when the TMMP is a control TMMP that includes only one of the at least two disulfide bonds present in the multiple disulfide-linked TMMP (e.g., a double disulfide-linked TMMP). In some cases, the percent of the total TMMP protein in the eluate that is non-aggregated disulfide-linked heterodimeric TMMP is at least 70%, at least 75%, at least 80%, at least 85%, at least 90%, at least 95%, or at least 99%. The protein A eluate can be subjected to size exclusion chromatography (SEC) and/or one or more other additional purification steps.

In some cases, a T-cell modulatory multimeric polypeptide of the present disclosure comprises at least one heterodimer comprising: a) a first polypeptide comprising: i) a MUC1 peptide, where the MUC1 peptide has a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, and peptides within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length); and ii) first MHC polypeptide; b) a second polypeptide comprising a second MHC polypeptide, and c) at least one immunomodulatory polypeptide, where the first and/or the second polypeptide comprises the immunomodulatory polypeptide, and where the heterodimer comprises at least two disulfide bonds (e.g., two disulfide bonds) between the first polypeptide and the second polypeptide (e.g., the heterodimer comprises: i) a first disulfide bond linking the first polypeptide and the second polypeptide; and ii) a second disulfide bond linking the first polypeptide and the second polypeptide). Expressed another way, the first polypeptide comprises a first Cys residue that forms a disulfide bond (a first disulfide bond) with a first Cys residue in the second polypeptide; and the first polypeptide comprises a second Cys residue that forms a disulfide bond (a second disulfide bond) with a second Cys residue in the second polypeptide.

In some cases, a TMMP of the present disclosure comprises: a) a first polypeptide comprising, in order from N-terminus to C-terminus: i) a MUC1 peptide; ii) a peptide linker; and iii) a β2M polypeptide; and b) a second polypeptide comprising an MHC Class I heavy chain polypeptide, where one or both of the first and the second polypeptides comprises at least one immunomodulatory polypeptide, where the TMMP comprises: a) a first disulfide linkage between: i) a Cys present in the linker between the MUC1 peptide and the β2M polypeptide; and ii) a first Cys introduced into the MHC Class I heavy chain polypeptide; and b) at least a second disulfide linkage between the first polypeptide and the second polypeptide, where the at least a second disulfide linkage is between: i) a Cys in the first polypeptide that is C-terminal to the Cys present in the linker; and ii) a Cys in the second polypeptide that is C-terminal to the first Cys introduced into the MHC Class I heavy chain polypeptide.

In some cases, a first and a second disulfide bond-forming Cys residues in a first or a second polypeptide of a TMMP of the present disclosure are from about 10 amino acids to about 200 amino acids apart from one another. For example, in some cases, a first and a second disulfide bond-forming Cys residues in a first or a second polypeptide of a TMMP are from about 10 amino acids (aa) to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 60 aa, from about 60 aa to about 70 aa, from about 70 aa to about 80 aa, from about 80 aa to about 90 aa, from about 90 aa to about 100 aa, from about 100 aa to about 110 aa, from about 110 aa to about 120 aa, from about 120 aa to about 130 aa, from about 130 aa to about 140 aa, from about 140 aa to about 150 aa, from about 150 aa to about 160 aa, from about 160 aa to about 170 aa, from about 170 aa to about 180 aa, from about 180 aa to about 190 aa, or from about 190 aa to about 200 aa.

As an example, in some cases, the first and second disulfide bond-forming Cys residues in the first polypeptide of a TMMP of the present disclosure are from about 10 amino acids to about 80 amino acid residues apart from one another. For example, in some cases, the second disulfide bond-forming Cys residue in the first polypeptide is from about 10 amino acids to about 80 amino acids (e.g., from about 10 amino acids (aa) to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 60 aa, from about 60 aa to about 70 aa, or from about 70 aa to about 80 aa) C-terminal to the first disulfide bond-forming Cys residue in the first polypeptide. In some cases, the second disulfide bond-forming Cys residue in the first polypeptide is 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, C-terminal to the first disulfide bond-forming Cys residue in the first polypeptide. In some cases, the second disulfide bond-forming Cys residue in the first polypeptide is 15 aa C-terminal to the first disulfide bond-forming Cys residue in the first polypeptide. In some cases, the second disulfide bond-forming Cys residue in the first polypeptide is 20 aa C-terminal to the first disulfide bond-forming Cys residue in the first polypeptide. In some cases, the second disulfide bond-forming Cys residue in the first polypeptide is 25 aa C-terminal to the first disulfide bond-forming Cys residue in the first polypeptide.

As another example, in some cases, the first and second disulfide bond-forming Cys residues in the second polypeptide of a TMMP of the present disclosure are from about 140 amino acids to about 160 amino acids apart from one another. For example, in some cases, the second disulfide bond-forming Cys residue in the second polypeptide is from about 140 amino acids to about 160 amino acids C-terminal to the first disulfide bond-forming Cys residue in the second polypeptide. In some cases, the second disulfide bond-forming Cys residue in the second polypeptide is 140 amino acids (aa), 141 aa, 142 aa, 143 aa, 144 aa, 145 aa, 146 aa, 147 aa, 148 aa, 149 aa, 150 aa, 151 aa, 152 aa, 153 aa, 154 aa, 155 aa, 156 aa, 157 aa, 158 aa, 159 aa, or 160 aa, C-terminal to the first disulfide bond-forming Cys residue in the second polypeptide.

A multiple disulfide-linked TMMP of the present disclosure (e.g., a double disulfide-linked TMMP) can comprise, for example: a) a first polypeptide comprising: i) a MUC1 peptide (e.g., a MUC1 peptide of from 4 amino acids to about 25 amino acids in length, that is bound by a TCR when the peptide is complexed with MHC polypeptides); and ii) a first MHC polypeptide, where the first polypeptide comprises a peptide linker between the MUC1 peptide and the first MHC polypeptide, where the peptide linker comprises a Cys residue, and where the first MHC polypeptide is a β2M polypeptide that comprises an amino acid substitution that introduces a Cys residue; b) and a second polypeptide comprising a second MHC polypeptide, where the second MHC polypeptide is a Class I heavy chain comprising a Y84C substitution and an A236C substitution, based on the amino acid numbering of HLA-A*0201 (depicted in FIG. 7A), or at corresponding positions in another Class I heavy chain allele, where the TMMP comprises a disulfide bond between the Cys residue in the peptide linker and the Cys residue at amino acid position 84 of the Class I heavy chain or corresponding position of another Class I heavy chain allele, and where the TMMP comprises a disulfide bond between the introduced Cys residue in the β2M polypeptide and the Cys at amino acid position 236 of the Class I heavy chain or corresponding position of another Class I heavy chain allele; and c) at least one immunomodulatory polypeptide, where the first and/or the second polypeptide comprises the at least one immunomodulatory polypeptide. Examples are depicted schematically in FIG. 12A and FIG. 12B.

In some cases, the peptide linker comprises the amino acid sequence GCGGS (SEQ ID NO:139). In some cases, the peptide linker comprises the amino acid sequence GCGGS(GGGGS)n (SEQ ID NO: 140), where n is an integer from 1 to 10. In some cases, the peptide linker comprises the amino acid sequence GCGGS(GGGGS)n (SEQ ID NO: 140), where n is 1. In some cases, the peptide linker comprises the amino acid sequence GCGGS(GGGGS)n (SEQ ID NO: 140), where n is 2. In some cases, the peptide linker comprises the amino acid sequence GCGGS(GGGGS)n (SEQ ID NO: 140), where n is 3. In some cases, the peptide linker comprises the amino acid sequence GCGGS(GGGGS)n (SEQ ID NO: 140), where n is 4. In some cases, the peptide linker comprises the amino acid sequence GCGGS(GGGGS)n (SEQ ID NO: 140), where n is 5. In some cases, the peptide linker comprises the amino acid sequence GCGGS(GGGGS)n (SEQ ID NO: 140), where n is 6. In some cases, the peptide linker comprises the amino acid sequence GCGGS(GGGGS)n (SEQ ID NO: 140), where n is 7. In some cases, the peptide linker comprises the amino acid sequence GCGGS(GGGGS)n (SEQ ID NO: 140), where n is 8. In some cases, the peptide linker comprises the amino acid sequence GCGGS(GGGGS)n (SEQ ID NO: 140), where n is 9. In some cases, the peptide linker comprises the amino acid sequence GCGGS(GGGGS)n (SEQ ID NO: 140), where n is 10.

In some cases, the peptide linker comprises the amino acid sequence CGGGS (SEQ ID NO:141). In some cases, the peptide linker comprises the amino acid sequence CGGGS(GGGGS)n (SEQ ID NO: 142), where n is an integer from 1 to 10. In some cases, the peptide linker comprises the amino acid sequence CGGGS(GGGGS)n (SEQ ID NO: 142), where n is 1. In some cases, the peptide linker comprises the amino acid sequence CGGGS(GGGGS)n (SEQ ID NO: 142), where n is 2. In some cases, the peptide linker comprises the amino acid sequence CGGGS(GGGGS)n (SEQ ID NO: 142), where n is 3. In some cases, the peptide linker comprises the amino acid sequence CGGGS(GGGGS)n (SEQ ID NO: 142), where n is 4. In some cases, the peptide linker comprises the amino acid sequence CGGGS(GGGGS)n (SEQ ID NO: 142), where n is 5. In some cases, the peptide linker comprises the amino acid sequence CGGGS(GGGGS)n (SEQ ID NO: 142), where n is 6. In some cases, the peptide linker comprises the amino acid sequence CGGGS(GGGGS)n (SEQ ID NO: 142), where n is 7. In some cases, the peptide linker comprises the amino acid sequence CGGGS(GGGGS)n (SEQ ID NO: 142), where n is 8. In some cases, the peptide linker comprises the amino acid sequence CGGGS(GGGGS)n (SEQ ID NO: 142), where n is 9. In some cases, the peptide linker comprises the amino acid sequence CGGGS(GGGGS)n (SEQ ID NO: 142), where n is 10.

The following are non-limiting examples of MHC Class I heavy chain comprising a Y84C substitution and an A236C substitution, based on the amino acid numbering of HLA-A*0201 (depicted in FIG. 7A), or at corresponding positions in another Class I heavy chain allele.

### HLA-A

In some cases, a multiple disulfide-linked TMMP of the present disclosure (e.g., a double disulfide-linked TMMP) comprises: a) a first polypeptide comprising: i) a MUC1 peptide (e.g., a MUC1 peptide of from 4 amino acids to 25 amino acids in length, that is bound by a TCR when the peptide is complexed with MHC polypeptides); and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; and b) a second polypeptide comprising an HLA-A MHC Class I heavy chain comprising an amino acid sequence having at least 60%, at least 70%, at least 80%, at least 90%, at least 95%, at least 98%, at least 99%, or 100%, amino acid sequence identity to the following amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWEP (SEQ ID NO:143), where amino acid 84 is a Cys and amino acid 236 is a Cys; and c) at least one immunomodulatory polypeptide, where the first and/or the second polypeptide comprises the at least one immunomodulatory polypeptide. In some cases, the peptide linker comprises the amino acid sequence GCGGS (SEQ ID NO:139). In some cases, the peptide linker comprises the amino acid sequence GCGGS(GGGGS)n (SEQ ID NO: 140), where n is an integer from 1 to 10. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:144), where amino acid 12 is a Cys. The at least one immunomodulatory polypeptide can be a cytokine, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, or a PD-L2 polypeptide. In some cases, the at least one immunomodulatory polypeptide is a reduced affinity variant, as described elsewhere herein. In some cases, the first or the second polypeptide comprises an Ig Fc polypeptide.

In some cases, a multiple disulfide-linked TMMP of the present disclosure (e.g., a double disulfide-linked TMMP) comprises an HLA-A Class I heavy chain polypeptide. In some cases, the HLA-A heavy chain polypeptide present in a multiple disulfide-linked TMMP of the present disclosure (e.g., a double disulfide-linked TMMP) comprises an amino acid sequence having at least 95%, at least 98%, or at least 99%, amino acid sequence identity to the HLA-A*0101, HLA-A*0201, HLA-A*0202, HLA-A*1101, HLA-A*2301, HLA-A*2402, HLA-A*2407, HLA-A*3303, or HLA-A*3401 amino acid sequence depicted in FIG. 7A, where the HLA-A heavy chain polypeptide comprises Y84C and A236C substitutions.

### HLA-A*0101 (Y84C; A236C)

In some cases, the HLA-A heavy chain polypeptide present in a multiple disulfide-linked TMMP of the present disclosure (e.g., a double disulfide-linked TMMMP) comprises an amino acid sequence having at least 95%, at least 98%, at least 99%, or 100%, amino acid sequence identity to the following HLA-A*0101 (Y84C; A236C) amino acid sequence:

GSHSMRYFFTSVSRPGRGEPRFIAVGYVDDTQFVRFDSDAASQKMEPRAPWIEQE GPEYWDQETRNMKAHSQTDRANLGTLRGCYNQSEDGSHTIQIMYGCDVGPDGRFLRGYRQDA YDGKDYIALNEDLRSWTAADMAAQITKRKWEAVHAAEQRRVYLEGRCVDGLRRYLENGKET LQRTDPPKTHMTHHPISDHEATLRCWALGFYPAEITLTWQRDGEDQTQDTELVETRPCGDGTF QKWAAVVVPSGEEQRYTCHVQHEGLPKPLTLRWE (SEQ ID NO:145), where amino acid 84 is a Cys and amino acid 236 is a Cys.

### HLA-A*0201 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*0201 (Y84C; A236C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWEP (SEQ ID NO:146), where amino acid 84 is a Cys and amino acid 236 is a Cys.

### HLA-A*0202 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*0202 (Y84C; A236C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWE (SEQ ID NO:147), where amino acid 84 is a Cys and amino acid 236 is a Cys.

### HLA-A*1101 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*1101 (Y84C; A236C) amino acid sequence: GSHSMRYFYTSVSRPGRGEPRFIAVGYVDDTQFVRFDSDAASQRMEPRAPWIEQEGPEYWDQE TRNVKAQSQTDRVDLGTLRGCYNQSEDGSHTIQIMYGCDVGPDGRFLRGYRQDAYDGKDYIA LNEDLRSWTAADMAAQITKRKWEAAHAAEQQRAYLEGRCVEWLRRYLENGKETLQRTDPPK THMTHHPISDHEATLRCWALGFYPAEITLTWQRDGEDQTQDTELVETRPCGDGTFQKWAAVV VPSGEEQRYTCHVQHEGLPKPLTLRWE (SEQ ID NO:148), where amino acid 84 is a Cys and amino acid 236 is a Cys.

### HLA-A*2301 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*2301 (Y84C; A236C) amino acid sequence: GSHSMRYFSTSVSRPGRGEPRFIAVGYVDDTQFVRFDSDAASQRMEPRAPWIEQEGPEYWDEET GKVKAHSQTDRENLRIALRCYNQSEAGSHTLQMMFGCDVGSDGRFLRGYHQYAYDGKDYIAL KEDLRSWTAADMAAQITQRKWEAARVAEQLRAYLEGTCVDGLRRYLENGKETLQRTDPPKTH MTHHPISDHEATLRCWALGFYPAEITLTWQRDGEDQTQDTELVETRPCGDGTFQKWAAVVVPS GEEQRYTCHVQHEGLPKPLTLRWE (SEQ ID NO:149), where amino acid 84 is a Cys and amino acid 236 is a Cys.

### HLA-A*2402 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*2402 (Y84C; A236C) amino acid sequence: GSHSMRYFSTSVSRPGRGEPRFIAVGYVDDTQFVRFDSDAASQRMEPRAPWIEQEGPEYWDEET GKVKAHSQTDRENLRIALRCYNQSEAGSHTLQMMFGCDVGSDGRFLRGYHQYAYDGKDYIAL KEDLRSWTAADMAAQITKRKWEAAHVAEQQRAYLEGTCVDGLRRYLENGKETLQRTDPPKT HMTHHPISDHEATLRCWALGFYPAEITLTWQRDGEDQTQDTELVETRPCGDGTFQKWAAVVV PSGEEQRYTCHVQHEGLPKPLTLRWE (SEQ ID NO:150), where amino acid 84 is a Cys and amino acid 236 is a Cys.

### HLA-A*2407 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*2407 (Y84C; A236C) amino acid sequence: GSHSMRYFSTSVSRPGRGEPRFIAVGYVDDTQFVRFDSDAASQRMEPRAPWIEQEGPEYWDEET GKVKAQSQTDRENLRIALRCYNQSEAGSHTLQMMFGCDVGSDGRFLRGYHQYAYDGKDYIAL KEDLRSWTAADMAAQITKRKWEAAHVAEQQRAYLEGTCVDGLRRYLENGKETLQRTDPPKT HMTHHPISDHEATLRCWALGFYPAEITLTWQRDGEDQTQDTELVETRPCGDGTFQKWAAVVV PSGEEQRYTCHVQHEGLPKPLTLRWE (SEQ ID NO:151), where amino acid 84 is a Cys and amino acid 236 is a Cys.

### HLA-A*3303 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*3303 (Y84C; A236C) amino acid sequence:

GSHSMRYFTTSVSRPGRGEPRFIAVGYVDDTQFVRFDSDAASQRMEPRAPWIEQE GPEYWDRNTRNVKAHSQIDRVDLGTLRGCYNQSEAGSHTIQMMYGCDVGSDGRFLRGYQQD AYDGKDYIALNEDLRSWTAADMAAQITQRKWEAARVAEQLRAYLEGTCVEWLRRYLENGKE TLQRTDPPKTHMTHHAVSDHEATLRCWALSFYPAEITLTWQRDGEDQTQDTELVETRPCGDGT FQKWASVVVPSGQEQRYTCHVQHEGLPKPLTLRWE (SEQ ID NO:152), where amino acid 84 is a Cys and amino acid 236 is a Cys.

### HLA-A*3401 (Y84C; A236C)

In some cases, the HLA-A heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-A*3401 (Y84C; A236C) amino acid sequence: GSHSMRYFYTSVSRPGRGEPRFIAVGYVDDTQFVRFDSDAASQRMEPRAPWIEQEGPEYWDRN TRKVKAQSQTDRVDLGTLRGCYNQSEDGSHTIQRMYGCDVGPDGRFLRGYQQDAYDGKDYIA LNEDLRSWTAADMAAQITQRKWETAHEAEQWRAYLEGTCVEWLRRYLENGKETLQRTDAPK THMTHHAVSDHEATLRCWALSFYPAEITLTWQRDGEDQTQDTELVETRPCGDGTFQKWASVV VPSGQEQRYTCHVQHEGLPKPLTLRWE (SEQ ID NO:153), where amino acid 84 is a Cys and amino acid 236 is a Cys.

### HLA-B

In some cases, a multiple disulfide-linked TMMP of the present disclosure (e.g., a double disulfide-linked TMMP) comprises: a) a first polypeptide comprising: i) a MUC1 peptide of at least 4 amino acids in length (e.g., a MUC1 peptide of from 4 amino acids to about 25 amino acids in length), that is bound by a TCR when the peptide is complexed with MHC polypeptides); and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; and b) a second polypeptide comprising an HLA-B MHC Class I heavy chain comprising an amino acid sequence having at least 60%, at least 70%, at least 80%, at least 90%, at least 95%, at least 98%, at least 99%, or 100%, amino acid sequence identity to the following amino acid sequence: GSHSMRYFYTSVSRPGRGEPRFISVGYVDDTQFVRFDSDAASPREEPRAPWIEQEGPEYWDRNT QIYKAQAQTDRESLRNLRGCYNQSEAGSHTLQSMYGCDVGPDGRLLRGHDQYAYDGKDYIAL NEDLRSWTAADTAAQITQRKWEAAREAEQRRAYLEGECVEWLRRYLENGKDKLERADPPKTH VTHHPISDHEATLRCWALGFYPAEITLTWQRDGEDQTQDTELVETRPCGDRTFQKWAAVVVPS GEEQRYTCHVQHEGLPKPLTLRWEP (SEQ ID NO:154), where amino acid 84 is a Cys and amino acid 236 is a Cys; and c) at least one immunomodulatory polypeptide, where the first and/or the second polypeptide comprises the at least one immunomodulatory polypeptide. In some cases, the peptide linker comprises the amino acid sequence GCGGS (SEQ ID NO:139). In some cases, the peptide linker comprises the amino acid sequence GCGGS(GGGGS)n (SEQ ID NO:140), where n is an integer from 1 to 10. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:155), where amino acid 12 is a Cys. The at least one immunomodulatory polypeptide can be a cytokine, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, or a PD-L2 polypeptide. In some cases, the at least one immunomodulatory polypeptide is a reduced affinity variant, as described elsewhere herein. In some cases, the first or the second polypeptide comprises an Ig Fc polypeptide.

In some cases, a multiple disulfide-linked TMMP of the present disclosure comprises an HLA-B Class I heavy chain polypeptide. In some cases, the HLA-B heavy chain polypeptide present in a multiple disulfide-linked TMMP of the present disclosure (e.g., a double disulfide-linked TMMP) comprises an amino acid sequence having at least 95%, at least 98%, or at least 99%, amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A, where the HLA-B heavy chain polypeptide comprises Y84C and A236C substitutions.

### HLA-B*0702 (Y84C; A236C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*0702 (Y84C; A236C) amino acid sequence:

GSHSMRYFYTSVSRPGRGEPRFISVGYVDDTQFVRFDSDAASPREEPRAPWIEQEG PEYWDRNTQIYKAQAQTDRESLRNLRGCYNQSEAGSHTLQSMYGCDVGPDGRLLRGHDQYAY DGKDYIALNEDLRSWTAADTAAQITQRKWEAAREAEQRRAYLEGECVEWLRRYLENGKDKLE RADPPKTHVTHHPISDHEATLRCWALGFYPAEITLTWQRDGEDQTQDTELVETRPCGDRTFQK WAAVVVPSGEEQRYTCHVQHEGLPKPLTLRWE (SEQ ID NO:156), where amino acid 84 is a Cys and amino acid 236 is a Cys.

### HLA-B*0801 (Y84C; A236C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*0801 (Y84C; A236C) amino acid sequence:

GSHSMRYFDTAMSRPGRGEPRFISVGYVDDTQFVRFDSDAASPREEPRAPWIEQE GPEYWDRNTQIFKTNTQTDRESLRNLRGCYNQSEAGSHTLQSMYGCDVGPDGRLLRGHNQYA YDGKDYIALNEDLRSWTAADTAAQITQRKWEAARVAEQDRAYLEGTCVEWLRRYLENGKDTL ERADPPKTHVTHHPISDHEATLRCWALGFYPAEITLTWQRDGEDQTQDTELVETRPCGDRTFQK WAAVVVPSGEEQRYTCHVQHEGLPKPLTLRWE (SEQ ID NO:157), where amino acid 84 is a Cys and amino acid 236 is a Cys.

### HLA-B*1502 (Y84C; A236C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*1502 (Y84C; A236C) amino acid sequence:

GSHSMRYFYTAMSRPGRGEPRFIAVGYVDDTQFVRFDSDAASPRMAPRAPWIEQE GPEYWDRNTQISKTNTQTYRESLRNLRGCYNQSEAGSHIIQRMYGCDVGPDGRLLRGYDQSAY DGKDYIALNEDLSSWTAADTAAQITQRKWEAAREAEQLRAYLEGLCVEWLRRYLENGKETLQ RADPPKTHVTHHPISDHEATLRCWALGFYPAEITLTWQRDGEDQTQDTELVETRPCGDRTFQK WAAVVVPSGEEQRYTCHVQHEGLPKPLTLRWE (SEQ ID NO:158), where amino acid 84 is a Cys and amino acid 236 is a Cys.

### HLA-B*3802 (Y84C; A236C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*3802 (Y84C; A236C) amino acid sequence:

GSHSMRYFYTSVSRPGRGEPRFISVGYVDDTQFVRFDSDAASPREEPRAPWIEQEG PEYWDRNTQICKTNTQTYRENLRTALRCYNQSEAGSHTLQRMYGCDVGPDGRLLRGHNQFAY DGKDYIALNEDLSSWTAADTAAQITQRKWEAARVAEQLRTYLEGTCVEWLRRYLENGKETLQ RADPPKTHVTHHPISDHEATLRCWALGFYPAEITLTWQRDGEDQTQDTELVETRPCGDRTFQK WAAVVVPSGEEQRYTCHVQHEGLPKPLTLRWE (SEQ ID NO:159), where amino acid 84 is a Cys and amino acid 236 is a Cys.

### HLA-B*4001 (Y84C; A2346C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*4001 (Y84C; A236C) amino acid sequence:

GSHSMRYFHTAMSRPGRGEPRFITVGYVDDTLFVRFDSDATSPRKEPRAPWIEQE GPEYWDRETQISKTNTQTYRESLRNLRGCYNQSEAGSHTLQRMYGCDVGPDGRLLRGHNQYA YDGKDYIALNEDLRSWTAADTAAQISQRKLEAARVAEQLRAYLEGECVEWLRRYLENGKDKL ERADPPKTHVTHHPISDHEATLRCWALGFYPAEITLTWQRDGEDQTQDTELVETRPCGDRTFQK WAAVVVPSGEEQRYTCHVQHEGLPKPLTLRWE (SEQ ID NO:160) where amino acid 84 is a Cys and amino acid 236 is a Cys.

### HLA-B*4601 (Y84C; A236C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*4601 (Y84C; A236C) amino acid sequence:

GSHSMRYFYTAMSRPGRGEPRFIAVGYVDDTQFVRFDSDAASPRMAPRAPWIEQE GPEYWDRETQKYKRQAQTDRVSLRNLRGCYNQSEAGSHTLQRMYGCDVGPDGRLLRGHDQS AYDGKDYIALNEDLSSWTAADTAAQITQRKWEAAREAEQWRAYLEGLCVEWLRRYLENGKET LQRADPPKTHVTHHPISDHEATLRCWALGFYPAEITLTWQRDGEDQTQDTELVETRPCGDRTFQ KWAAVVVPSGEEQRYTCHVQHEGLPKPLTLRWE (SEQ ID NO:161) where amino acid 84 is a Cys and amino acid 236 is a Cys.

### HLA-B*5301 (Y84C; A236C)

In some cases, the HLA-B heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-B*5301 (Y84C; A236C) amino acid sequence:

GSHSMRYFYTAMSRPGRGEPRFIAVGYVDDTQFVRFDSDAASPRTEPRAPWIEQE GPEYWDRNTQIFKTNTQTYRENLRIALRCYNQSEAGSHIIQRMYGCDLGPDGRLLRGHDQSAY DGKDYIALNEDLSSWTAADTAAQITQRKWEAARVAEQLRAYLEGLCVEWLRRYLENGKETLQ RADPPKTHVTHHPVSDHEATLRCWALGFYPAEITLTWQRDGEDQTQDTELVETRPCGDRTFQK WAAVVVPSGEEQRYTCHVQHEGLPKPLTLRWE (SEQ ID NO:162) where amino acid 84 is a Cys and amino acid 236 is a Cys.

### HLA-C

In some cases, a multiple disulfide-linked TMMP of the present disclosure (e.g., a double disulfide-linked TMMP) comprises: a) a first polypeptide comprising: i) a MUC1 peptide of at least 4 amino acids in length (e.g., a MUC1 peptide of from 4 amino acids to about 25 amino acids in length) that is bound by a TCR when the peptide is complexed with MHC polypeptides); and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; and b) a second polypeptide comprising an HLA-C MHC Class I heavy chain comprising an amino acid sequence having at least 60%, at least 70%, at least 80%, at least 90%, at least 95%, at least 98%, at least 99%, or 100%, amino acid sequence identity to the following amino acid sequence: CSHSMRYFDTAVSRPGRGEPRFISVGYVDDTQFVRFDSDAASPRGEPRAPWVEQEGPEYWDRE TQNYKRQAQADRVSLRNLRGCYNQSEDGSHTLQRMYGCDLGPDGRLLRGYDQSAYDGKDYI ALNEDLRSWTAADTAAQITQRKLEAARAAEQLRAYLEGTCVEWLRRYLENGKETLQRAEPPKT HVTHHPLSDHEATLRCWALGFYPAEITLTWQRDGEDQTQDTELVETRPCGDGTFQKWAAVVV PSGQEQRYTCHMQHEGLQEPLTLSWEP (SEQ ID NO:163), where amino acid 84 is a Cys and amino acid 236 is a Cys; and c) at least one immunomodulatory polypeptide, where the first and/or the second polypeptide comprises the at least one immunomodulatory polypeptide. In some cases, the peptide linker comprises the amino acid sequence GCGGS (SEQ ID NO:139). In some cases, the peptide linker comprises the amino acid sequence GCGGS(GGGGS)n (SEQ ID NO: 140), where n is an integer from 1 to 10. In some cases, the β2M polypeptide comprises an R12C substitution. For example, the β2M polypeptide can comprises an amino acid sequence having at least 90%, at least 95%, at least 98%, at least 99%, or 100%, amino acid sequence identity to the following amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:164), where amino acid 12 is a Cys. The at least one immunomodulatory polypeptide can be a cytokine, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, or a PD-L2 polypeptide. In some cases, the at least one immunomodulatory polypeptide is a reduced affinity variant, as described elsewhere herein. In some cases, the first or the second polypeptide comprises an Ig Fc polypeptide.

In some cases, a multiple disulfide-linked TMMP of the present disclosure (e.g., a double disulfide-linked TMMP) comprises an HLA-C Class I heavy chain polypeptide. In some cases, the HLA-C heavy chain polypeptide present in a multiple disulfide-linked TMMP of the present disclosure (e.g., a double disulfide-linked TMMP) comprises an amino acid sequence having at least 95%, at least 98%, or at least 99%, amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 amino acid sequence depicted in FIG. 9A, where the HLA-C heavy chain polypeptide comprises Y84C and A236C substitutions.

### HLA-C*01:02 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*01:02 (Y84C; A236C) amino acid sequence:

CSHSMKYFFTSVSRPGRGEPRFISVGYVDDTQFVRFDSDAASPRGEPRAPWVEQE GPEYWDRETQKYKRQAQTDRVSLRNLRGCYNQSEAGSHTLQWMCGCDLGPDGRLLRGYDQY AYDGKDYIALNEDLRSWTAADTAAQITQRKWEAAREAEQRRAYLEGTCVEWLRRYLENGKET LQRAEHPKTHVTHHPVSDHEATLRCWALGFYPAEITLTWQWDGEDQTQDTELVETRPCGDGTF QKWAAVMVPSGEEQRYTCHVQHEGLPEPLTLRWEP (SEQ ID NO:165), where amino acid 84 is a Cys and amino acid 236 is a Cys.

### HLA-C*0303 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*03:03 (Y84C; A236C) amino acid sequence:

GSHSMRYFYTAVSRPGRGEPHFIAVGYVDDTQFVRFDSDAASPRGEPRAPWVEQE GPEYWDRETQKYKRQAQTDRVSLRNLRGCYNQSEARSHIIQRMYGCDVGPDGRLLRGYDQYA YDGKDYIALNEDLRSWTAADTAAQITQRKWEAAREAEQLRAYLEGLCVEWLRRYLKNGKETL QRAEHPKTHVTHHPVSDHEATLRCWALGFYPAEITLTWQWDGEDQTQDTELVETRPCGDGTF QKWAAVVVPSGEEQRYTCHVQHEGLPEPLTLRWEP (SEQ ID NO:166), where amino acid 84 is a Cys and amino acid 236 is a Cys.

### HLA-C*0304 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*03:04 (Y84C; A236C) amino acid sequence:

GSHSMRYFYTAVSRPGRGEPHFIAVGYVDDTQFVRFDSDAASPRGEPRAPWVEQE GPEYWDRETQKYKRQAQTDRVSLRNLRGCYNQSEAGSHIIQRMYGCDVGPDGRLLRGYDQYA YDGKDYIALNEDLRSWTAADTAAQITQRKWEAAREAEQLRAYLEGLCVEWLRRYLKNGKETL QRAEHPKTHVTHHPVSDHEATLRCWALGFYPAEITLTWQWDGEDQTQDTELVETRPCGDGTF QKWAAVVVPSGEEQRYTCHVQHEGLPEPLTLRWEP (SEQ ID NO:167), where amino acid 84 is a Cys and amino acid 236 is a Cys.

### HLA-C*0401 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*04:01 (Y84C; A236C) amino acid sequence:

GSHSMRYFSTSVSWPGRGEPRFIAVGYVDDTQFVRFDSDAASPRGEPREPWVEQE GPEYWDRETQKYKRQAQADRVNLRKLRGCYNQSEDGSHTLQRMFGCDLGPDGRLLRGYNQF AYDGKDYIALNEDLRSWTAADTAAQITQRKWEAAREAEQRRAYLEGTCVEWLRRYLENGKET LQRAEHPKTHVTHHPVSDHEATLRCWALGFYPAEITLTWQWDGEDQTQDTELVETRPCGDGTF QKWAAVVVPSGEEQRYTCHVQHEGLPEPLTLRWKP (SEQ ID NO:168), where amino acid 84 is a Cys and amino acid 236 is a Cys.

### HLA-C*0602 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*06:02 (Y84C; A236C) amino acid sequence:

CSHSMRYFDTAVSRPGRGEPRFISVGYVDDTQFVRFDSDAASPRGEPRAPWVEQE GPEYWDRETQKYKRQAQADRVNLRKLRGCYNQSEDGSHTLQWMYGCDLGPDGRLLRGYDQS AYDGKDYIALNEDLRSWTAADTAAQITQRKWEAAREAEQWRAYLEGTCVEWLRRYLENGKE TLQRAEHPKTHVTHHPVSDHEATLRCWALGFYPAEITLTWQRDGEDQTQDTELVETRPCGDGT FQKWAAVVVPSGEEQRYTCHVQHEGLPEPLTLRWEP (SEQ ID NO:169), where amino acid 84 is a Cys and amino acid 236 is a Cys.

### HLA-C*0701 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*07:01 (Y84C; A236C) amino acid sequence:

CSHSMRYFDTAVSRPGRGEPRFISVGYVDDTQFVRFDSDAASPRGEPRAPWVEQE GPEYWDRETQNYKRQAQADRVSLRNLRGCYNQSEDGSHTLQRMYGCDLGPDGRLLRGYDQS AYDGKDYIALNEDLRSWTAADTAAQITQRKLEAARAAEQLRAYLEGTCVEWLRRYLENGKET LQRAEPPKTHVTHHPLSDHEATLRCWALGFYPAEITLTWQRDGEDQTQDTELVETRPCGDGTF QKWAAVVVPSGQEQRYTCHMQHEGLQEPLTLSWEP (SEQ ID NO:170), where amino acid 84 is a Cys and amino acid 236 is a Cys.

### HLA-C*0702 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*07:02 (Y84C; A236C) amino acid sequence:

CSHSMRYFDTAVSRPGRGEPRFISVGYVDDTQFVRFDSDAASPRGEPRAPWVEQE GPEYWDRETQKYKRQAQADRVSLRNLRGCYNQSEDGSHTLQRMSGCDLGPDGRLLRGYDQS AYDGKDYIALNEDLRSWTAADTAAQITQRKLEAARAAEQLRAYLEGTCVEWLRRYLENGKET LQRAEPPKTHVTHHPLSDHEATLRCWALGFYPAEITLTWQRDGEDQTQDTELVETRPCGDGTF QKWAAVVVPSGQEQRYTCHMQHEGLQEPLTLSWEP (SEQ ID NO:171), where amino acid 84 is a Cys and amino acid 236 is a Cys.

### HLA-C*0801 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*08:01 (Y84C; A236C) amino acid sequence:

CSHSMRYFYTAVSRPGRGEPRFIAVGYVDDTQFVQFDSDAASPRGEPRAPWVEQE GPEYWDRETQKYKRQAQTDRVSLRNLRGCYNQSEAGSHTLQRMYGCDLGPDGRLLRGYNQF AYDGKDYIALNEDLRSWTAADTAAQITQRKWEAARTAEQLRAYLEGTCVEWLRRYLENGKKT LQRAEHPKTHVTHHPVSDHEATLRCWALGFYPAEITLTWQRDGEDQTQDTELVETRPCGDGTF QKWAAVVVPSGEEQRYTCHVQHEGLPEPLTLRWGP (SEQ ID NO:172), where amino acid 84 is a Cys and amino acid 236 is a Cys.

### HLA-C*1502 (Y84C; A236C)

In some cases, the HLA-C heavy chain polypeptide present in a multiple disulfide-linked TMMP of the present disclosure (e.g., a double disulfide-linked TMMP) comprises an amino acid sequence having at least 95%, at least 98%, at least 99%, or 100%, amino acid sequence identity to the following HLA-C*15:02 (Y84C; A236C) amino acid sequence:

CSHSMRYFYTAVSRPGRGEPHFIAVGYVDDTQFVRFDSDAASPRGEPRAPWVEQE GPEYWDRETQNYKRQAQTDRVNLRKLRGCYNQSEAGSHIIQRMYGCDLGPDGRLLRGHDQLA YDGKDYIALNEDLRSWTAADTAAQITQRKWEAAREAEQLRAYLEGTCVEWLRRYLENGKETL QRAEHPKTHVTHHPVSDHEATLRCWALGFYPAEITLTWQRDGEDQTQDTELVETRPCGDGTFQ KWAAVVVPSGEEQRYTCHVQHEGLPEPLTLRWEP (SEQ ID NO:173), where amino acid 84 is a Cys and amino acid 236 is a Cys.

### Scaffold Polypeptides

A TMMP can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:59), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMMP, compared to a control TMMP lacking the scaffold polypeptide. For example, in some cases, a scaffold polypeptide increases the in vivo half-life (e.g., the serum half-life) of the TMMP, compared to a control TMMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMMP, compared to a control TMMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

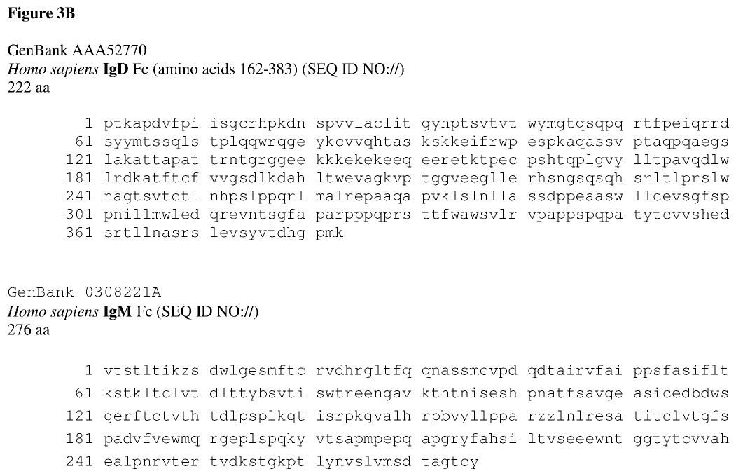

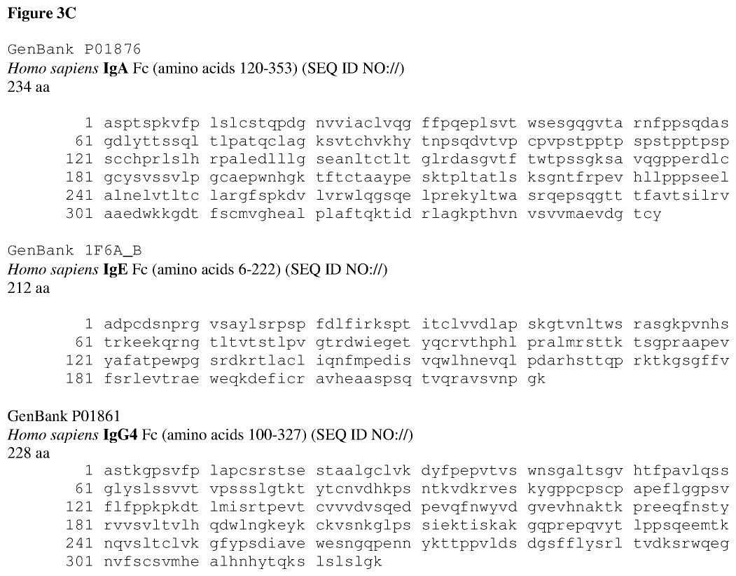

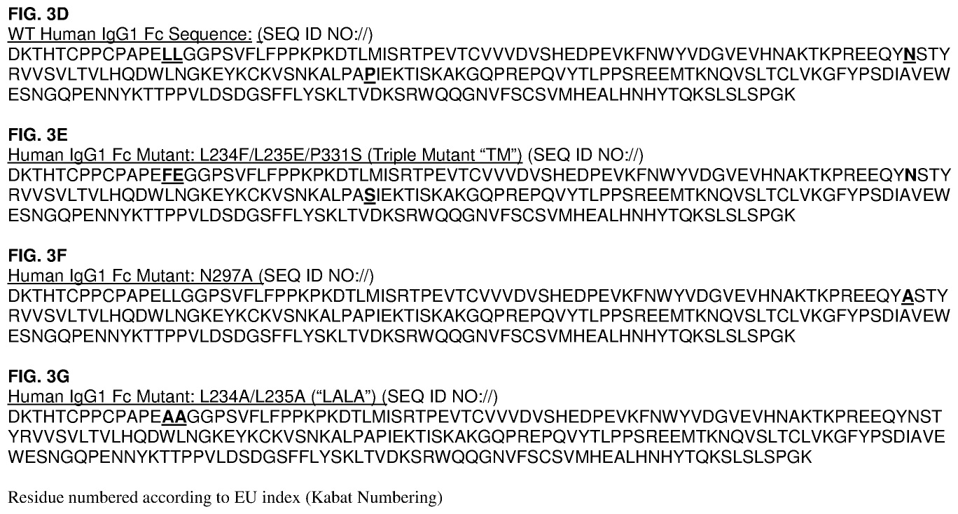

In some cases, the first and/or the second polypeptide chain of a TMMP of the present disclosure comprises an Fc polypeptide. The Fc polypeptide of a TMMP of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 3A-3G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A; and comprises a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 3B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 3B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 3C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 3C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 3C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 3C.

In some cases, the IgG4 Fc polypeptide comprises the following amino acid sequence:

(SEQ ID NO: 175)
PPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSPG.

In some cases, the Fc polypeptide present in a TMMP comprises the amino acid sequence depicted in FIG. 3A (human IgG1 Fc). In some cases, the Fc polypeptide present in a TMMP comprises the amino acid sequence depicted in FIG. 3A (human IgG1 Fc), except for a substitution of N297 (N77 of the amino acid sequence depicted in FIG. 3A) with an amino acid other than asparagine. In some cases, the Fc polypeptide present in a TMMP comprises the amino acid sequence depicted in FIG. 3C (human IgG1 Fc comprising an N297A substitution, which is N77 of the amino acid sequence depicted in FIG. 3A). In some cases, the Fc polypeptide present in a TMMP comprises the amino acid sequence depicted in FIG. 3A (human IgG1 Fc), except for a substitution of L234 (L14 of the amino acid sequence depicted in FIG. 3A) with an amino acid other than leucine. In some cases, the Fc polypeptide present in a TMMP comprises the amino acid sequence depicted in FIG. 3A (human IgG1 Fc), except for a substitution of L235 (L15 of the amino acid sequence depicted in FIG. 3A) with an amino acid other than leucine.

In some cases, the Fc polypeptide present in a TMMP comprises the amino acid sequence depicted in FIG. 3E. In some cases, the Fc polypeptide present in a TMMP comprises the amino acid sequence depicted in FIG. 3F. In some cases, the Fc polypeptide present in a TMMP comprises the amino acid sequence depicted in FIG. 5G (human IgG1 Fc comprising an L234A substitution and an L235A substitution, corresponding to positions 14 and 15 of the amino acid sequence depicted in FIG. 3G). In some cases, the Fc polypeptide present in a TMMP comprises the amino acid sequence depicted in FIG. 3A (human IgG1 Fc), except for a substitution of P331 (P111 of the amino acid sequence depicted in FIG. 3A) with an amino acid other than proline; in some cases, the substitution is a P331S substitution. In some cases, the Fc polypeptide present in a TMMP comprises the amino acid sequence depicted in FIG. 3A (human IgG1 Fc), except for substitutions at L234 and L235 (L14 and L15 of the amino acid sequence depicted in FIG. 3A) with amino acids other than leucine. In some cases, the Fc polypeptide present in a TMMP comprises the amino acid sequence depicted in FIG. 3A (human IgG1 Fc), except for substitutions at L234 and L235 (L14 and L15 of the amino acid sequence depicted in FIG. 3A) with amino acids other than leucine, and a substitution of P331 (P111 of the amino acid sequence depicted in FIG. 3A) with an amino acid other than proline. In some cases, the Fc polypeptide present in a TMMP comprises the amino acid sequence depicted in FIG. 3E (human IgG1 Fc comprising L234F, L235E, and P331S substitutions (corresponding to amino acid positions 14, 15, and 111 of the amino acid sequence depicted in FIG. 3E). In some cases, the Fc polypeptide present in a TMMP is an IgG1 Fc polypeptide that comprises L234A and L235A substitutions (substitutions of L14 and L15 of the amino acid sequence depicted in FIG. 3A with Ala), as depicted in FIG. 3G.

### Tumor-Targeting Polypeptide

A TMMP of the present disclosure can include a tumor-targeting polypeptide (TTP). For example, a TTP can be specific for a MUC1 polypeptide present on a cancer cell. In some cases, the TTP is specific for the cleaved form of MUC1; see, e.g., Fessler et al. (2009) Breast Cancer Res. Treat. 118:113. A TTP can be specific for a cancer-associated antigen other than MUC1. In some cases, the TTP is an antibody. In some cases, the TTP is a single-chain T-cell receptor (scTCR).

In some cases, the TTP is an antibody. In some cases, the TTP is a single-chain antibody. In some cases, the TTP is a scFv. In some cases, the TTP is a nanobody (also referred to as a single domain antibody (sdAb)). In some cases, the TTP is a heavy chain nanobody. In some cases, the TTP is a light chain nanobody. In some cases, the TTP is an antibody specific for a glycosylated MUC1 peptide; see, e.g., Naito et al. (2017) ACS Omega 2:7493; and U.S. Pat. No. 10,017,580. In some cases, the TTP is an antibody specific for a cancer-associated antigen other than MUC1.

### Anti-MUC1 Antibodies

As one non-limiting example, a TTP can be a single-chain Fv specific for MUC1. See, e.g., Singh et al. (2007) Mol. Cancer Ther. 6:562; Thie et al. (2011) PLoSOne 6:e15921; Imai et al. (2004) Leukemia 18:676; Posey et al. (2016) Immunity 44:1444; EP3130607; EP3164418; WO 2002/044217; and US 2018/0112007. In some cases, a TTP is a scFv specific for the MUC1 peptide VTSAPDTRPAPGSTAPPAHG (SEQ ID NO:319). In some cases, a TTP is a scFv specific for the MUC1 peptide SNIKFRPGSVVVQLTLAFREGTINVHDVETQFNQYKTEAASRY (SEQ ID NO:320). In some cases, a TTP is a scFv specific for the MUC1 peptide SVVVQLTLAFREGTINVHDVETQFNQYKTEAASRY (SEQ ID NO:321). In some cases, a TTP is a scFv specific for the MUC1 peptide LAFREGTINVHDVETQFNQY (SEQ ID NO:322). In some cases, a TTP is a scFv specific for the MUC1 peptide SNIKFRPGSVVVQLTLAAFREGTIN (SEQ ID NO:323).

As an example, an anti-MUC1 antibody can comprise: a VH CDR1 having the amino acid sequence RYGMS (SEQ ID NO:324); a VH CDR2 having the amino acid sequence TISGGGTYIYYPDSVKG (SEQ ID NO:325); a VH CDR3 having the amino acid sequence DNYGRNYDYGMDY (SEQ ID NO:326); a VL CDR1 having the amino acid sequence SATSSVSYIH (SEQ ID NO:327); a VL CDR2 having the amino acid sequence STSNLAS (SEQ ID NO:328); and a VL CDR3 having the amino acid sequence QQRSSSPFT (SEQ ID NO:329). See, e.g., US 2018/0112007.

As another example, an anti-MUC1 antibody can comprise a VH CDR1 having the amino acid sequence GYAMS (SEQ ID NO:330); a VH CDR2 having the amino acid sequence TISSGGTYIYYPDSVKG (SEQ ID NO:331); a VH CDR3 having the amino acid sequence LGGDNYYEYFDV (SEQ ID NO:332); a VL CDR1 having the amino acid sequence RASKSVSTSGYSYMH (SEQ ID NO:333); a VL CDR2 having the amino acid sequence LASNLES (SEQ ID NO:334); and a VL CDR3 having the amino acid sequence QHSRELPFT (SEQ ID NO:335). See, e.g., US 2018/0112007.

As another example, an anti-MUC1 antibody can comprise a VH CDR1 having the amino acid sequence DYAMN (SEQ ID NO:336); a VH CDR2 having the amino acid sequence VISTFSGNINFNQKFKG (SEQ ID NO:337); a VH CDR3 having the amino acid sequence SDYYGPYFDY (SEQ ID NO:338); a VL CDR1 having the amino acid sequence RSSQTIVHSNGNTYLE (SEQ ID NO:339); a VL CDR2 having the amino acid sequence KVSNRFS (SEQ ID NO:340); and a VL CDR3 having the amino acid sequence (FQGSHVPFT (SEQ ID NO:341). See, e.g., US 2018/0112007.

As another example, an anti-MUC1 antibody can comprise a VH CDR1 having the amino acid sequence GYAMS (SEQ ID NO:342); a VH CDR2 having the amino acid sequence TISSGGTYIYYPDSVKG (SEQ ID NO:343); a VH CDR3 having the amino acid sequence LGGDNYYEY (SEQ ID NO:344); a VL CDR1 having the amino acid sequence TASKSVSTSGYSYMH (SEQ ID NO:345); a VL CDR2 having the amino acid sequence LVSNLES (SEQ ID NO:346); and a VL CDR3 having the amino acid sequence QHIRELTRSE (SEQ ID NO:347). See, e.g., US 2018/0112007.

### Targets

As noted above, in some cases, a TTP present in a TMMP of the present disclosure can be specific for MUC1, or a cancer-associated antigen (“CAA”) other than MUC1. In some cases, the target of a TTP is a peptide/HLA (pHLA) complex on the surface of a cancer cell, where the peptide can be a cancer-associated peptide (e.g., a peptide fragment of a cancer-associated antigen).

### CAAs

CAAs that can be targeted with a tumor-targeting polypeptide present in a TMPP of the present disclosure include, e.g., NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, myelin, NKG2D (Natural Killer Group 2D) ligands, PSMA (prostate specific membrane antigen), and ROR1 (type I receptor tyrosine kinase-like orphan receptor).

CAAs that can be targeted with a TTP present in a TMPP of the present disclosure include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Le^{gamma}, macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, ituximabrv ituximabrvivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the CAA is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the CAA is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the CAA is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma} Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

### Peptide/HLA Complexes

In some cases, the target of a TTP is a peptide/HLA (pHLA) complex on the surface of a cancer cell, where the peptide can be a cancer-associated peptide (e.g., a peptide fragment of a cancer-associated antigen). Cancer-associated peptides are known in the art. In some cases, a cancer-associated peptide is bound to a HLA complex comprising an HLA-A*0201 heavy chain and a β2M polypeptide.

In some cases, the peptide epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the peptide epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the peptide epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, a cancer-associated peptide is a peptide of at least 4 amino acids in length, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of any one of the following cancer-associated antigens: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypepti ituximabrvivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, a Fos-related antigen-1 (FOSL) polypeptide; a human papilloma virus (HPV) antigen; an alpha-feto protein (AFP) antigen; and a Wilms tumor-1 (WT1) antigen.

For example, in some cases, a TTP present in a TMMP of the present disclosure binds to: a) a WT-1 peptide bound to an HLA complex comprising an HLA heavy chain (e.g., an HLA-A*0201 heavy chain or an HLA-A*2402 heavy chain) and a β2M polypeptide; b) an HPV peptide bound to an HLA complex comprising a class I HLA heavy chain and a β2M polypeptide; c) a mesothelin peptide bound to an HLA complex comprising a class I HLA heavy chain and a β2M polypeptide; d) a Her2 peptide bound to an HLA complex comprising a class I HLA heavy chain and a β2M polypeptide; or e) a BCMA peptide bound to an HLA complex comprising a class I HLA heavy chain and a β2M polypeptide.

In some cases, a cancer-associated peptide is a peptide of at least 4 amino acids in length, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a mesothelin polypeptide having at least 90%, at least 95%, at least 98%, at least 99%, or 100%, amino acid sequence identity to the following mesothelin amino acid sequence:

LAGE TGQEAAPLDG VLANPPNISS LSPRQLLGFP CAEVSGLSTE RVRELAVALA QKNVKLSTEQ LRCLAHRLSE PPEDLDALPL DLLLFLNPDA FSGPQACTRF FSRITKANVD LLPRGAPERQ RLLPAALACW GVRGSLLSEA DVRALGGLAC DLPGRFVAES AEVLLPRLVS CPGPLDQDQQ EAARAALQGG GPPYGPPSTW SVSTMDALRG LLPVLGQPII RSIPQGIVAA WRQRSSRDPS WRQPERTILR PRFRREVEKT ACPSGKKARE IDESLIFYKK WELEACVDAA LLATQMDRVN AIPFTYEQLD VLKHKLDELY PQGYPESVIQ HLGYLFLKMS PEDIRKWNVT SLETLKALLE VNKGHEMSPQ VATLIDRFVK GRGQLDKDTL DTLTAFYPGY LCSLSPEELS SVPPSSIWAV RPQDLDTCDP RQLDVLYPKA RLAFQNMNGS EYFVKIQSFL GGAPTEDLKA LSQQNVSMDL ATFMKLRTDA VLPLTVAEVQ KLLGPHVEGL KAEERHRPVR DWILRQRQDD LDTLGLGLQG GIPNGYLVLD LSMQEALSGT PCLLGPGPVL TVLALLLAST LA (SEQ ID NO:176). For example, a mesothelin peptide present in a pHLA complex can be: i) KLLGPHVEGL (SEQ ID NO:526); ii) AFYPGYLCSL (SEQ ID NO:177), which can bind HLA-A*2402/02M; iii) VLPLTVAEV (SEQ ID NO:178); iv) ELAVALAQK (SEQ ID NO:179); v) ALQGGGPPY (SEQ ID NO:180); vi) FYPGYLCSL (SEQ ID NO:181); vii) LYPKARLAF (SEQ ID NO:182); viii) LLFLLFSLGWVGPSR (SEQ ID NO:183); ix) VNKGHEMSPQAPRRP (SEQ ID NO:184); x) FMKLRTDAVLPLTVA (SEQ ID NO:185); or xi) DAALLATQMD (SEQ ID NO:186).

In some cases, a cancer-associated peptide is a peptide of at least 4 amino acids in length, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a Her2 polypeptide having at least 90%, at least 95%, at least 98%, at least 99%, or 100%, amino acid sequence identity to the following Her2 (receptor tyrosine-protein kinase erbB2) amino acid sequence:

(SEQ ID NO: 187)
MELAALCRWG LLLALLPPGA ASTQVCTGTD MKLRLPASPE
THLDMLRHLY QGCQVVQGNL ELTYLPTNAS LSFLQDIQEV
QGYVLIAHNQ VRQVPLQRLR IVRGTQLFED NYALAVLDNG
DPLNNTTPVT GASPGGLREL QLRSLTEILK GGVLIQRNPQ
LCYQDTILWK DIFHKNNQLA LTLIDTNRSR ACHPCSPMCK
GSRCWGESSE DCQSLTRTVC AGGCARCKGP LPTDCCHEQC
AAGCTGPKHS DCLACLHFNH SGICELHCPA LVTYNTDTFE
SMPNPEGRYT FGASCVTACP YNYLSTDVGS CTLVCPLHNQ
EVTAEDGTQR CEKCSKPCAR VCYGLGMEHL REVRAVTSAN
IQEFAGCKKI FGSLAFLPES FDGDPASNTA PLQPEQLQVF
ETLEEITGYL YISAWPDSLP DLSVFQNLQV IRGRILHNGA
YSLTLQGLGI SWLGLRSLRE LGSGLALIHH NTHLCFVHTV
PWDQLFRNPH QALLHTANRP EDECVGEGLA CHQLCARGHC
WGPGPTQCVN CSQFLRGQEC VEECRVLQGL PREYVNARHC
LPCHPECQPQ NGSVTCFGPE ADQCVACAHY KDPPFCVARC
PSGVKPDLSY MPIWKFPDEE GACQPCPINC THSCVDLDDK
GCPAEQRASP LTSIISAVVG ILLVVVLGVV FGILIKRRQQ
KIRKYTMRRL LQETELVEPL TPSGAMPNQA QMRILKETEL
RKVKVLGSGA FGTVYKGIWI PDGENVKIPV AIKVLRENTS
PKANKEILDE AYVMAGVGSP YVSRLLGICL TSTVQLVTQL
MPYGCLLDHV RENRGRLGSQ DLLNWCMQIA KGMSYLEDVR
LVHRDLAARN VLVKSPNHVK ITDFGLARLL DIDETEYHAD
GGKVPIKWMA LESILRRRFT HQSDVWSYGV TVWELMTFGA
KPYDGIPARE IPDLLEKGER LPQPPICTID VYMIMVKCWM
IDSECRPRFR ELVSEFSRMA RDPQRFVVIQ NEDLGPASPL
DSTFYRSLLE DDDMGDLVDA EEYLVPQQGF FCPDPAPGAG
GMVHHRHRSS STRNM.

In some cases, a cancer-associated peptide is a peptide of at least 4 amino acids in length, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a BCMA polypeptide having at least 90%, at least 95%, at least 98%, at least 99%, or 100%, amino acid sequence identity to the following BCMA amino acid sequence:

(SEQ ID NO: 188)
MLQMAGQCSQ NEYFDSLLHA CIPCQLRCSS NTPPLTCQRY
CNASVTNSVK GTNAILWTCL GLSLIISLAV FVLMFLLRKI
SSEPLKDEFK NTGSGLLGMA NIDLEKSRTG DEIILPRGLE
YTVEECTCED CIKSKPKVDS DHCFPLPAME EGATILVTTK
TNDYCKSLPA ALSATEIEKS ISAR.

In some cases, a cancer-associated peptide is a peptide of at least 4 amino acids in length, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a WT-1 polypeptide having at least 90%, at least 95%, at least 98%, at least 99%, or 100%, amino acid sequence identity to the following WT-1 amino acid sequence:

(SEQ ID NO: 189)
MDFLLLQDPA STCVPEPASQ HTLRSGPGCL QQPEQQGVRD
PGGIWAKLGA AEASAERLQG RRSRGASGSE PQQMGSDVRD
LNALLPAVPS LGGGGGCALP VSGAAQWAPV LDFAPPGASA
YGSLGGPAPP PAPPPPPPPP PHSFIKQEPS WGGAEPHEEQ
CLSAFTVHFS GQFTGTAGAC RYGPFGPPPP SQASSGQARM
FPNAPYLPSC LESQPAIRNQ GYSTVTFDGT PSYGHTPSHH
AAQFPNHSFK HEDPMGQQGS LGEQQYSVPP PVYGCHTPTD
SCTGSQALLL RTPYSSDNLY QMTSQLECMT WNQMNLGATL
KGHSTGYESD NHTTPILCGA QYRIHTHGVF RGIQDVRRVP
GVAPTLVRSA SETSEKRPFM CAYPGCNKRY FKLSHLQMHS
RKHTGEKPYQ CDFKDCERRF SRSDQLKRHQ RRHTGVKPFQ
CKTCQRKFSR SDHLKTHTRT HTGEKPFSCR WPSCQKKFAR
SDELVRHHNM HQRNMTKLQL AL.

Non-limiting examples of WT-1 peptides include RMFPNAPYL (SEQ ID NO:190), CMTWNQMN (SEQ ID NO:191), CYTWNQMNL (SEQ ID NO:192), CMTWNQMNLGATLKG (SEQ ID NO:193), WNQMNLGATLKGVAA (SEQ ID NO:194), CMTWNYMNLGATLKG (SEQ ID NO:195), WNYMNLGATLKGVAA (SEQ ID NO:196), MTWNQMNLGATLKGV (SEQ ID NO:197), TWNQMNLGATLKGVA (SEQ ID NO:198), CMTWNLMNLGATLKG (SEQ ID NO:199), MTWNLMNLGATLKGV (SEQ ID NO:200), TWNLMNLGATLKGVA (SEQ ID NO:201), WNLMNLGATLKGVAA (SEQ ID NO:202), MNLGATLK (SEQ ID NO:203), MTWNYMNLGATLKGV (SEQ ID NO:204), TWNYMNLGATLKGVA (SEQ ID NO:205), CMTWNQMNLGATLKGVA (SEQ ID NO:206), CMTWNLMNLGATLKGVA (SEQ ID NO:207), CMTWNYMNLGATLKGVA (SEQ ID NO:208), GYLRNPTAC (SEQ ID NO:209), GALRNPTAL (SEQ ID NO:210), YALRNPTAC (SEQ ID NO:211), GLLRNPTAC (SEQ ID NO:212), RYRPHPGAL (SEQ ID NO:213), YQRPHPGAL (SEQ ID NO:214), RLRPHPGAL (SEQ ID NO:215), RIRPHPGAL (SEQ ID NO:216), QFPNHSFKHEDPMGQ (SEQ ID NO:217), HSFKHEDPY (SEQ ID NO:218), QFPNHSFKHEDPM (SEQ ID NO:219), QFPNHSFKHEDPY (SEQ ID NO:220), KRPFMCAYPGCNK (SEQ ID NO:221), KRPFMCAYPGCYK (SEQ ID NO:222), FMCAYPGCY (SEQ ID NO:223), FMCAYPGCK (SEQ ID NO:224), KRPFMCAYPGCNKRY (SEQ ID NO:225), SEKRPFMCAYPGCNK (SEQ ID NO:226), KRPFMCAYPGCYKRY (SEQ ID NO:227), NLMNLGATL (SEQ ID NO:228), and NYMNLGATL (SEQ ID NO:229).In some cases, a cancer-associated peptide is a peptide of at least 4 amino acids in length, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of an HPV polypeptide having at least 90%, at least 95%, at least 98%, at least 99%, or 100%, amino acid sequence identity to a human papilloma virus (HPV) peptide. An HPV peptide can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. Non-limiting examples of HPV peptides include: E6 18-26 (KLPQLCTEL; SEQ ID NO:230); E6 26-34 (LQTTIHDII; SEQ ID NO:231); E6 49-57 (VYDFAFRDL; SEQ ID NO:232); E6 52-60 (FAFRDLCIV; SEQ ID NO:233); E6 75-83 (KFYSKISEY; SEQ ID NO:234); E6 80-88 (ISEYRHYCY; SEQ ID NO:235); E7 7-15 (TLHEYMLDL; SEQ ID NO:236); E7 11-19 (YMLDLQPET; SEQ ID NO:237); E7 44-52 (QAEPDRAHY; SEQ ID NO:238); E7 49-57 (RAHYNIVTF (SEQ ID NO:239); E7 61-69 (CDSTLRLCV; SEQ ID NO:240); and E7 67-76 (LCVQSTHVDI; SEQ ID NO:241); E7 82-90 (LLMGTLGIV; SEQ ID NO:242); E7 86-93 (TLGIVCPI; SEQ ID NO:243); and E7 92-93 (LLMGTLGIVCPI; SEQ ID NO:244).

### Antibodies

As noted above, in some cases, a TTP present in a TMMP of the present disclosure is an antibody. In some cases, the TTP is an antibody that is specific for a CAA. In some cases, the TTP is an antibody specific for a peptide/HLA complex on the surface of a cancer cell, where the peptide can be a cancer-associated peptide (e.g., a peptide fragment of a cancer-associated antigen).

Non-limiting examples of CAA-targeted antibodies that can be included in a TMMP of the present disclosure include, but are not limited to, abituzumab (anti-CD51), LL1 (anti-CD74), LL2 or RFB4 (anti-CD22), veltuzumab (hA20; anti-CD20), rituximab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), R1 (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), ofatumumab (anti-CD20), and anetumab (anti-mesothelin).

In some cases, the tumor-targeting polypeptide is an antibody. In some cases, the tumor-targeting polypeptide is a single-chain antibody. In some cases, the tumor-targeting polypeptide is a scFv. In some cases, the tumor-targeting polypeptide is a nanobody (also referred to as a single domain antibody (sdAb)). In some cases, the tumor-targeting polypeptide is a heavy chain nanobody. In some cases, the tumor-targeting polypeptide is a light chain nanobody.

VH and VL amino acid sequences of various tumor antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of tumor antigen-binding antibodies.

### Anti-Her2

In some cases, an anti-Her2 antibody comprises: a) a light chain comprising an amino acid sequence having at least 90%, at least 95%, at least 98%, at least 99%, or 100%, amino acid sequence identity to the following amino acid sequence:

DIQMTQSPSSLSASVGDRVTITCRASQDVNTAVAWYQQKPGKAPKLLIYSASFLYS GVPSRFSGSRSGTDFTLTISSLQPEDFATYYCQQHYTTPPTFGQGTKVEIKRTVAAPSVFIFPPSDE QLKSGTASVVCLLNNFYPREAKVQWKVDNALQSGNSQESVTEQDSKDSTYSLSSTLTLSKADY EKHKVYACEVTHQGLSSPVTKSFNRGEC (SEQ ID NO:248); and b) a heavy chain comprising an amino acid sequence having at least 90%, at least 95%, at least 98%, at least 99%, or 100%, amino acid sequence identity to the following amino acid sequence:

(SEQ ID NO: 249)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:250); and b) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:251). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:252); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:253). Suitable linkers are described elsewhere herein and include, e.g., (GGGGS)n (SEQ ID NO: 254), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 2, above; and Kabat 1991). In some cases, the V_{H} and V_{L} CDRs are as defined by Chothia (see, e.g., Table 2, above; and Chothia 1987).

For example, an anti-Her2 antibody can comprise a VL CDR1 having the amino acid sequence RASQDVNTAVA (SEQ ID NO:255); a VL CDR2 having the amino acid sequence SASFLY (SEQ ID NO:256); a VL CDR3 having the amino acid sequence QQHYTTPP (SEQ ID NO:257); a VH CDR1 having the amino acid sequence GFNIKDTY (SEQ ID NO:258); a VH CDR2 having the amino acid sequence IYPTNGYT (SEQ ID NO:259); and a VH CDR3 having the amino acid sequence SRWGGDGFYAMDY (SEQ ID NO:260).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 261)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSGGGGSGGGGSGGGGSDIQMTQSPSSLSASV
GDRVTITCRASQDVNTAVAWYQQKPGKAPKLLIYSASFLYSGVPSRFSGS
RSGTDFTLTISSLQPEDFATYYCQQHYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:

DIQMTQSPSSLSASVGDRVTITCKASQDVSIGVAWYQQKPGKAPKLLIYSASYRYT GVPSRFSGSGSGTDFTLTISSLQPEDFATYYCQQYYIYPYTFGQGTKVEIKRTVAAPSVFIFPPSDE QLKSGTASVVCLLNNFYPREAKVQWKVDNALQSGNSQESVTEQDSKDSTYSLSSTLTLSKADY EKHKVYACEVTHQGLSSPVTKSFNRGEC (SEQ ID NO:262); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 263)
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO:264); and b) a VH comprising an amino acid sequence having at least 90%, at least 95%, at least 98%, at least 99%, or 100%, amino acid sequence identity to the amino acid sequence:

(SEQ ID NO: 265)
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 2, above; and Kabat 1991). In some cases, the V_{H} and V_{L} CDRs are as defined by Chothia (see, e.g., Table 2, above; and Chothia 1987).

For example, an anti-HER2 antibody can comprise a VL CDR1 having the amino acid sequence KASQDVSIGVA (SEQ ID NO:266); a VL CDR2 having the amino acid sequence SASYRY (SEQ ID NO:267); a VL CDR3 having the amino acid sequence QQYYIYPY (SEQ ID NO:268); a VH CDR1 having the amino acid sequence GFTFTDYTMD (SEQ ID NO:269); a VH CDR2 having the amino acid sequence ADVNPNSGGSIYNQRFKG (SEQ ID NO:270); and a VH CDR3 having the amino acid sequence ARNLGPSFYFDY (SEQ ID NO:271).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 272)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSGGGGSGGGGSGGGGSDIQMTQSPSSLSASV
GDRVTITCRASQDVNTAVAWYQQKPGKAPKLLIYSASFLYSGVPSRFSGS
RSGTDFTLTISSLQPEDFATYYCQQHYTTPPTFGQGTKVEIK.

### Anti-CD19

Anti-CD19 antibodies are known in the art; and the VH and VL, or the VH and VL CDRs, of any anti-CD19 antibody can be used in a TMMP of the present disclosure. See e.g., WO 2005/012493.

In some cases, an anti-CD19 antibody includes a VL CDR1 comprising the amino acid sequence KASQSVDYDGDSYLN (SEQ ID NO:273); a VL CDR2 comprising the amino acid sequence DASNLVS (SEQ ID NO:274); and a VL CDR3 comprising the amino acid sequence QQSTEDPWT (SEQ ID NO:275). In some cases, an anti-CD19 antibody includes a VH CDR1 comprising the amino acid sequence SYWMN (SEQ ID NO:276); a VH CDR2 comprising the amino acid sequence QIWPGDGDTNYNGKFKG (SEQ ID NO:277); and a VH CDR3 comprising the amino acid sequence RETTTVGRYYYAMDY (SEQ ID NO:278). In some cases, an anti-CD19 antibody includes a VL CDR1 comprising the amino acid sequence KASQSVDYDGDSYLN (SEQ ID NO:279); a VL CDR2 comprising the amino acid sequence DASNLVS (SEQ ID NO:280); a VL CDR3 comprising the amino acid sequence QQSTEDPWT (SEQ ID NO:281); a VH CDR1 comprising the amino acid sequence SYWMN (SEQ ID NO:282); a VH CDR2 comprising the amino acid sequence QIWPGDGDTNYNGKFKG (SEQ ID NO:283); and a VH CDR3 comprising the amino acid sequence RETTTVGRYYYAMDY (SEQ ID NO:284).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 285)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVS.

### Anti-Mesothelin

Anti-mesothelin antibodies are known in the art; and the VH and VL, or the VH and VL CDRs, of any anti-mesothelin antibody can be used in a TMMP of the present disclosure. See, e.g., U.S. 2019/0000944; WO 2009/045957; WO 2014/031476; U.S. Pat. No. 8,460,660; US 2013/0066055; and WO 2009/068204.

In some cases, an anti-mesothelin antibody comprises: a) a light chain comprising an amino acid sequence having at least 90%, at least 95%, at least 98%, at least 99%, or 100%, amino acid sequence identity to the following amino acid sequence:

(SEQ ID NO: 286)
DIALTQPASVSGSPGQSITISCTGTSSDIGGYNSVSWYQQHPGKAPKLMI
YGVNNRPSGVSNRFSGSKSGNTASLTISGLQAEDEADYYCSSYDIESATP
VFGGGTKLTVLGQPKAAPSVTLFPPSSEELQANKATLVCLISDFYPGAVT
VAWKGDSSPVKAGVETTTPSKQSNNKYAASSYLSLTPEQWKSHRSYSCQV
THEGSTVEKTVAPTESS;

and

b) a heavy chain comprising an amino acid sequence having at least 90%, at least 95%, at least 98%, at least 99%, or 100%, amino acid sequence identity to the following amino acid sequence:

(SEQ ID NO: 287)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO: 288); and b) a VH comprising an amino acid sequence having at least 90%, at least 95%, at least 98%, at least 99%, or 100%, amino acid sequence identity to the amino acid sequence:

(SEQ ID NO: 289)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 2, above; and Kabat 1991). In some cases, the V_{H} and V_{L} CDRs are as defined by Chothia (see, e.g., Table 2, above; and Chothia 1987).

For example, an anti-mesothelin antibody can comprise a VL CDR1 having the amino acid sequence TGTSSDIGGYNSVS (SEQ ID NO:290); a VL CDR2 having the amino acid sequence LMIYGVNNRPS (SEQ ID NO:291); a VL CDR3 having the amino acid sequence SSYDIESATP (SEQ ID NO:292); a VH CDR1 having the amino acid sequence GYSFTSYWIG (SEQ ID NO:293); a VH CDR2 having the amino acid sequence WMGIIDPGDSRTRYSP (SEQ ID NO:294); and a VH CDR3 having the amino acid sequence GQLYGGTYMDG (SEQ ID NO:295).

An anti-mesothelin antibody can be a scFv. As one non-limiting example, an anti-mesothelin scFv can comprise the following amino acid sequence:

(SEQ ID NO: 296)
QVQLQQSGAEVKKPGASVKVSCKASGYTFTGYYMHWVRQAPGQGLEWMGR
INPNSGGTNYAQKFQGRVTMTRDTSISTAYMELSRLRSEDTAVYYCARGR
YYGMDVWGQGTMVTVSSGGGGSGGGGSGGGGSGGGGSEIVLTQSPATLSL
SPGERATISCRASQSVSSNFAWYQQRPGQAPRLLIYDASNRATGIPPRFS
GSGSGTDFTLTISSLEPED FAAYYCHQRSNWLYTFGQGTKVDIK,

where VH CDR1, CDR2, and CDR3 are underlined; and VL CDR1, CDR2, and CDR3 are bolded and underlined.

As one non-limiting example, an anti-mesothelin scFv can comprise the following amino acid sequence:

(SEQ ID NO: 297)
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
DLRRTVVTPRAYYGMDVWGQGTTVTVSSGGGGSGGGGSGGGGSGGGGSD
IQLTQSPSTLSASVGDRVTITCQASQDISNSLNWYQQKAGKAPKLLIY
DASTLETGVPSRFSGSGSGTDFSF
TISSLQPEDIATYYCQQHDNLPLTFGQGTKVEIK,

where VH CDR1, CDR2, and CDR3 are underlined; and VL CDR1, CDR2, and CDR3 are bolded and underlined.

### Anti-BCMA

Anti-BCMA (B-cell maturation antigen) antibodies are known in the art; and the VH and VL, or the VH and VL CDRs, of any anti-BCMA antibody can be used in a TMMP of the present disclosure. See, e.g., WO 2014/089335; and US 2019/0153061.

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 298)
QSVLTQPPSASGTPGQRVTISCSGSSSNIGSNTVNWYQQLPGTAPKLLIF
NYHQRPSGVPDRFSGSKSGSSASLAISGLQSEDEADYYCAAWDDSLNGWV
FGGGTKLTVLGQPKAAPSVTLFPPSSEELQANKATLVCLISDFYPGAVTV
AWKADSSPVKAGVETTTPDSKQSNNKYAASSYLSLTPEQWKSHRSYSCQV
THEGSTVEKTVAPTECS;

and

b) a heavy chain comprising an amino acid sequence having at least 90%, at least 95%, at least 98%, at least 99%, or 100%, amino acid sequence identity to the following amino acid sequence

(SEQ ID NO: 299)
EVQLVESGGGLVKPGGSLRLSCAASGFTFGDYALSWFRQAPGKGLEWVGV
SRSKAYGGTTDYAASVKGRFTISRDDSKSTAYLQMNSLKTEDTAVYYCAS
SGYSSGWTPFDYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK.

In some cases, an anti-mesothelin antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:

QSVLTQPPSASGTPGQRVTISCSGSSSNIGSNTVNWYQQLPGTAPKLLIFNYHQRPS GVPDRFSGSKSGSSASLAISGLQSEDEADYYCAAWDDSLNGWVFGGGTKLTVLG (SEQ ID NO:300); and b) a VH comprising an amino acid sequence having at least 90%, at least 95%, at least 98%, at least 99%, or 100%, amino acid sequence identity to the amino acid sequence:

(SEQ ID NO: 301)
EVQLVESGGGLVKPGGSLRLSCAASGFTFGDYALSWFRQAPGKGLEWVGV
SRSKAYGGTTDYAASVKGRFTISRDDSKSTAYLQMNSLKTEDTAVYYCAS
SGYSSGWTPFDYWGQGTLVTVSSASTKGPSV.

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 2, above; and Kabat 1991). In some cases, the V_{H} and V_{L} CDRs are as defined by Chothia (see, e.g., Table 2, above; and Chothia 1987).

For example, an anti-BCMA antibody can comprise a VL CDR1 having the amino acid sequence SSNIGSNT (SEQ ID NO:302), a VL CDR2 having the amino acid sequence NYH, a VL CDR3 having the amino acid sequence AAWDDSLNGWV (SEQ ID NO:303)), a VH CDR1 having the amino acid sequence GFTFGDYA (SEQ ID NO:304), a VH CDR2 having the amino acid sequence SRSKAYGGTT (SEQ ID NO:305), and a VH CDR3 having the amino acid sequence ASSGYSSGWTPFDY (SEQ ID NO:306).

An anti-BCMA antibody can be a scFv. As one non-limiting example, an anti-BCMA scFv can comprise the following amino acid sequence:

(SEQ ID NO: 307)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYNGYDVLDNWGQGTLVTVSSGGGGSGGGGSGGGGSGGGGSDIQMTQSPS
SLSASVGDRVTITCSASQDISNYLNWYQQKPGKAPKLLIYYTSNLHSGVP
SRFSGSGSGTDFTLTISSLQPEDFATYYCQQYRKLPWTFGQGTKLEIKR.

As another example, an anti-BCMA scFv can comprise the following amino acid sequence:

(SEQ ID NO: 308)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKRGGGGSGGGGSGGGGSGGGGSQVQLVQSGAEVKKPGSSVKVSC
KASGGTFSNYWMHWVRQAPGQGLEWMGATYRGHSDTYYNQKFKGRVTITA
DKSTSTAYMELSSLRSEDTAVYYCARGAIYNGYDVLDNWGQGTLVTVSS.

### Single-Chain T-Cell Receptors

As noted above, in some cases, a TTP present in a TMMP of the present disclosure is a scTCR. A TTP can be a scTCR specific for a peptide/HLA complex on the surface of a cancer cell, where the peptide can be a cancer-associated peptide (e.g., a peptide fragment of a cancer-associated antigen). Amino acid sequences of scTCRs specific for cancer-associated peptides bound to an HLA complex are known in the art. See, e.g., US 2019/0135914; US 2019/0062398; and US 2018/0371049.

A scTCR includes an alpha chain variable region (Vα) and a beta chain variable region (Vβ) covalently linked through a suitable peptide linker sequence. For example, the Vα can be covalently linked to the Vβ through a suitable peptide linker (L) sequence fused to the C-terminus of the Vα and the N-terminus of the Vβ. An scTCR can have the structure Vα-L-Vβ. An scTCR can have the structure Vβ-L-Vα. An scTCR can also comprise a constant domain (also referred to as constant region). In some cases, an scTCR comprises, in order from N-terminus to C-terminus: i) a TCR α chain variable domain polypeptide; ii) a peptide linker; iii) a TCR β chain variable domain polypeptide; and iv) a TCR β chain constant region extracellular domain polypeptide. In some cases, an scTCR comprises, in order from N-terminus to C-terminus: i) a TCR β chain variable domain polypeptide; ii) a peptide linker; iii) a TCR α chain variable domain polypeptide; and iv) a TCR α chain constant region extracellular domain polypeptide.

Amino acid sequences of scTCRs specific for peptide/HLA complexes, where the peptide is a cancer-associated peptide, are known in the art. See, e.g., US 2019/0135914; US 2019/0062398; US 2018/0371049; US 2019/0144563; and US 2019/0119350.

For example, a scTCR can be specific for an NY-ESO epitope such as an SLLMWITQC peptide bound to an HLA complex comprising an HLA-A*0201 heavy chain and a β2M polypeptide. As an example, such an scTCR can comprise: i) a TCR α chain variable region comprising an amino acid sequence having at least 90%, at least 95%, at least 98%, at least 99%, or 100%, amino acid sequence identity to the amino acid sequence: MQEVTQIPAALSVPEGENLVLNCSFTDSAIYNLQWFRQDPGKGLTSLLLIQSSQREQTSGRLNAS LDKSSGRSTLYIAASQPGDSATYLCAVRPTSGGSYIPTFGRGTSLIVHPY (SEQ ID NO:353), where amino acid 20 can be V or A; amino acid 51 can be Q, P, S, T, or M; amino acid 52 can be S, P, F, or G, amino acid 53 can be S, W, H, or T; amino acid 94 can be P, H, or A; amino acid 95 can be T, L, M, A, Q, Y, E, I, F, V, N, G, S, D, or R; amino acid 96 can be S, L, T, Y, I, Q, V, E, A, W, R, G, H, D, or K; amino acid 97 can be G, D, N, V, S, T, or A; amino acid 98 can be G, P, H, S, T, W, or A; amino acid 99 can be S, T, Y, D, H, V, N, E, G, Q, K, A, I, or R; amino acid 100 can be Y, F, M, or D; amino acid 101 can be I, P, T, or M; and amino acid 103 can be T or A; and ii) a TCR β chain variable region comprising an amino acid sequence having at least 90%, at least 95%, at least 98%, at least 99%, or 100%, amino acid sequence identity to the amino acid sequence:

(SEQ ID NO: 354)
MGVTQTPKFQVLKTGQSMTLQCAQDMNHEYMSWYRQDPGMGLRLIHYSVG
AGITDQGEVPNGYNVSRSTTEDFPLRLLSAAPSQTSVYFCASSYVGNTGE
LFFGEGSRLTVL,

where amino acid 18 can be M or V; amino acid 50 can be G, V, or I; amino acid 52 can be G or Q; amino acid 53 can be I, T, or M; amino acid 55 can be D or R; amino acid 56 can be Q or R; amino acid 70 can be T or I; amino acid 94 can be Y, N, or F; amino acid 95 can be V or L; and amino acid 97 can be N, G, or D. For example, in some cases, a scTCR can comprise: i) a TCR α chain variable region comprising the amino acid sequence: MQEVTQIPAALSVPEGENLVLNCSFTDSAIYNLQWFRQDPGKGLTSLLLIMSHQREQTSGRLNA SLDKSSGRSTLYIAASQPGDSATYLCAVRPTSGGSYIPTFGRGTSLIVHPY (SEQ ID NO:355); and a TCR β chain variable region comprising the amino acid sequence:

(SEQ ID NO: 356)
MGVTQTPKFQVLKTGQSMTLQCAQDMNHEYMSWYRQDPGMGLRLIHYSVS
AGITDQGEVPNGYNVSRSTTEDFPLRLLSAAPSQTSVYFCASSYVGNTGE
LFFGEGSRLTVL.

As another example, a scTCR can be specific for an HPV peptide epitope (e.g., an HPV peptide of the amino acid sequence YIIFVYIPL (HPV 16 E5₆₃₋₇₁; SEQ ID NO:357), KLPQLCTEL (HPV 16 E6₁₁₋₁₉; SEQ ID NO:358), TIHEIILECV (HPV 16 E6; SEQ ID NO:359), YMLDLQPET (HPV 16 E7₁₁₋₁₉; SEQ ID NO:360), TLGIVCPI (HPV 16 E7₈₆₋₉₃; SEQ ID NO: 361), KCIDFYSRI (HPV 18 E6₆₇₋₇₅; SEQ ID NO:362), or FQQLFLNTL (HPV 18 E7₈₆₋₉₄; SEQ ID NO:363)) bound to an HLA complex comprising an HL heavy chain and a β2M polypeptide. As an example, such an scTCR can comprise: i) a TCR α chain variable region comprising an amino acid sequence having at least 90%, at least 95%, at least 98%, at least 99%, or 100%, amino acid sequence identity to the amino acid sequence: METLLGLLILQLQLQWVSSKQEVTQIPAALSVPEGENLVLNCSFTDSAIYNLQWFRQDPGKGLT SLLLIQSSQREQTSGRLNASLDKSSGRSTLYIAASQPGDSATYLCAVRETSGSRLTFGEGTQLTVN PD (SEQ ID NO:364); and ii) a TCR β chain variable region comprising an amino acid sequence having at least 90%, at least 95%, at least 98%, at least 99%, or 100%, amino acid sequence identity to the amino acid sequence:

(SEQ ID NO: 365)
MGIRLLCRVAFCFLAVGLVDVKVTQSSRYLVKRTGEKVFLECVQDMDHEN
MFWYRQDPGLGLRLIYFSYDVKMKEKGDIPEGYSVSREKKERFSLILESA
STNQTSMYLCASSFWGRSTDTQYFGPGTRLTVL.

Exemplary configurations The TTP can be present on the first polypeptide and/or the second polypeptide of a TMMP of the present disclosure. Thus, a TMMP of the present disclosure can comprise at least one heterodimer comprising: a) a first polypeptide comprising: i) a peptide epitope; and ii) first MHC polypeptide; b) a second polypeptide comprising a second MHC polypeptide, c) at least one immunomodulatory polypeptide, where the first and/or the second polypeptide comprises the immunomodulatory polypeptide; d) an Ig Fc polypeptide or a non-Ig scaffold, where the first and/or the second polypeptide comprises the Ig Fc polypeptide or the non-Ig scaffold; and e) a tumor-targeting polypeptide (TTP), where the first and/or the second polypeptide comprises the TTP. These components can be arranged in any of a variety of configurations. For example, in some cases, a TMMP of the present disclosure comprises: a) a first polypeptide comprising, in order from N-terminus to C-terminus: i) a MUC1 peptide; ii) a first MHC polypeptide; and iii) at least one immunomodulatory polypeptide; and b) a second polypeptide comprising, in order from N-terminus to C-terminus: i) a second MHC polypeptide; ii) an Ig Fc polypeptide; and iii) a TTP. As another example, in some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) at least one immunomodulatory polypeptide; ii) a second MHC polypeptide; iii) an Ig Fc polypeptide; and iv) a TTP. As another example, in some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) a second MHC polypeptide; ii) an Ig Fc polypeptide; iii) at least one immunomodulatory polypeptide; and iv) a TTP. As another example, in some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) at least one immunomodulatory polypeptide; ii) a second MHC polypeptide; iii) an Ig Fc polypeptide; and iv) a TTP. As another example, in some cases, a TMMP of the present disclosure comprises: a) a first polypeptide comprising, in order from N-terminus to C-terminus: i) at least one immunomodulatory polypeptide; ii) a MUC1 peptide; and iii) a first MHC polypeptide; and b) a second polypeptide comprising, in order from N-terminus to C-terminus: i) a second MHC polypeptide; ii) an Ig Fc polypeptide; and iii) a TTP. As another example, in some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) a second MHC polypeptide; ii) at least one immunomodulatory polypeptide; iii) an Ig Fc polypeptide; and iv) a TTP.

### Linkers

A TMMP of the present disclosure can include one or more linkers, where the one or more linkers are between one or more of: i) an MHC Class I polypeptide and an Ig Fc polypeptide, where such a linker is referred to herein as “L1”; ii) an immunomodulatory polypeptide and an MHC Class I polypeptide, where such a linker is referred to herein as “L2”; iii) a first immunomodulatory polypeptide and a second immunomodulatory polypeptide, where such a linker is referred to herein as “L3”; iv) a peptide antigen (“epitope”) and an MHC Class I polypeptide; v) an MHC Class I polypeptide and a dimerization polypeptide (e.g., a first or a second member of a dimerizing pair); and vi) a dimerization polypeptide (e.g., a first or a second member of a dimerizing pair) and an IgFc polypeptide.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

Exemplary linkers include glycine polymers (G)ₙ, glycine-serine polymers (including, for example, (GS)ₙ, (GSGGS)ₙ (SEQ ID NO:366) and (GGGS)ₙ (SEQ ID NO:367), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:368), GGSGG (SEQ ID NO: 369), GSGSG (SEQ ID NO:370), GSGGG (SEQ ID NO:371), GGGSG (SEQ ID NO:372), GSSSG (SEQ ID NO:373), and the like. Exemplary linkers can include, e.g., Gly(Ser₄)n (SEQ ID NO:374), where n is 1, 2, 3, 4, 5, 6, 7, 8, 9, or 10. In some cases, a linker comprises the amino acid sequence (GSSSS)n (SEQ ID NO:375), where n is 4. In some cases, a linker comprises the amino acid sequence (GSSSS)n (SEQ ID NO:376), where n is 5. In some cases, a linker comprises the amino acid sequence (GGGGS)n (SEQ ID NO:377), where n is 1. In some cases, a linker comprises the amino acid sequence (GGGGS)n (SEQ ID NO:378), where n is 2. In some cases, a linker comprises the amino acid sequence (GGGGS)n (SEQ ID NO:379), where n is 3. In some cases, a linker comprises the amino acid sequence (GGGGS)n (SEQ ID NO:380), where n is 4. In some cases, a linker comprises the amino acid sequence (GGGGS)n (SEQ ID NO:381), where n is 5. In some cases, a linker comprises the amino acid sequence (GGGGS)n (SEQ ID NO:382), where n is 6. In some cases, a linker comprises the amino acid sequence (GGGGS)n (SEQ ID NO:383), where n is 7, In some cases, a linker comprises the amino acid sequence (GGGGS)n (SEQ ID NO:384), where n is 8, In some cases, a linker comprises the amino acid sequence (GGGGS)n (SEQ ID NO:385), where n is 9, In some cases, a linker comprises the amino acid sequence (GGGGS)n (SEQ ID NO:386), where n is 10. In some cases, a linker comprises the amino acid sequence AAAGG (SEQ ID NO:387).

In some cases, a linker polypeptide, present in a first polypeptide of a TMMP of the present disclosure, includes a cysteine residue that can form a disulfide bond with a cysteine residue present in a second polypeptide of a TMMP of the present disclosure. In some cases, for example, a suitable linker comprises the amino acid sequence GCGGSGGGGSGGGGS (SEQ ID NO:388). As another example, a suitable linker can comprise the amino acid sequence GCGGS(G4S)n (SEQ ID NO:389), where n is 1, 2, 3, 4, 5, 6, 7, 8, or 9. For example, in some cases, the linker comprises the amino acid sequence GCGGSGGGGSGGGGSGGGGS (SEQ ID NO:390). As another example, the linker comprises the amino acid sequence GCGGSGGGGSGGGGS (SEQ ID NO:391).

### MUC1 Peptides

As discussed above, a TMMP of the present disclosure comprises a MUC1 peptide that is typically at least about 4 amino acids in length, and presents a MUC1 epitope to a T cell when in an MHC/peptide complex (e.g., an HLA/peptide complex).

A MUC1 peptide present in a TMMP of the present disclosure can have a length of at least 4 amino acids, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length).

A MUC1 epitope present in a TMMP of the present disclosure is a peptide specifically bound by a T-cell, i.e., the epitope is specifically bound by an epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10¹⁰ M.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following MUC1 amino acid sequence (full length human MUC1 isoform 1 protein precursor (isoform 1, Uniprot P15941-1)):

(SEQ ID NO: 573)
MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRTAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDNRPALGS TAPPVHNVTS
ASGSASGSAS TLVHNGTSAR ATTTPASKST PFSIPSHHSD
TPTTLASHST KTDASSTHHS SVPPLTSSNH STSPQLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA TSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 2 amino acid sequence:

(SEQ ID NO: 574)
MTPGTQSPFF LLLLLTVLTA TTAPKPATVV TGSGHASSTP
GGEKETSATQ RSSVPSSTEK NAFNSSLEDP STDYYQELQR
DISEMFLQIY KQGGFLGLSN IKFRPGSVVV QLTLAFREGT
INVHDVETQF NQYKTEAASR YNLTISDVSV SDVPFPFSAQ
SGAGVPGWGI ALLVLVCVLV ALAIVYLIAL AVCQCRRKNY
GQLDIFPARD TYHPMSEYPT YHTHGRYVPP SSTDRSPYEK
VSAGNGGSSL SYTNPAVAAT SANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 3 amino acid sequence:

(SEQ ID NO: 575)
MTPGTQSPFF LLLLLTVLTV VTGSGHASST PGGEKETSAT
QRSSVPSSTE KNAFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA TSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 5 amino acid sequence:

(SEQ ID NO: 576)
MTPGTQSPFF LLLLLTVLTV VTGSGHASST PGGEKETSAT
QRSSVPSSTE KNAIPAPTTT KSCRETFLKC FCRFINKGVF
WASPILSSVS DVPFPFSAQS GAGVPGWGIA LLVLVCVLVA
LAIVYLIALA VCQCRRKNYG QLDIFPARDT YHPMSEYPTY
HTHGRYVPPS STDRSPYEKV SAGNGGSSLS YTNPAVAATS ANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 6 amino acid sequence:

(SEQ ID NO: 577)
MTPGTQSPFF LLLLLTVLTV VTGSGHASST PGGEKETSAT
QRSSVPSSTE KNAFNSSLED PSTDYYQELQ RDISEMAVCQ
CRRKNYGQLD IFPARDTYHP MSEYPTYHTH GRYVPPSSTD
RSPYEKVSAG NGGSSLSYTN PAVAATSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 7 amino acid sequence:

(SEQ ID NO: 578)
MTPGTQSPFF LLLLLTVLTA TTAPKPATVV TGSGHASSTP
GGEKETSATQ RSSVPSSTEK NAFNSSLEDP STDYYQELQR
DISEMAVCQC RRKNYGQLDI FPARDTYHPM SEYPTYHTHG
RYVPPSSTDR SPYEKVSAGN GGSSLSYTNP AVAATSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 8 amino acid sequence:

(SEQ ID NO: 579)
MTPGTQSPFF LLLLLTVLTV VTGSGHASST PGGEKETSAT
QRSSVPSSTE KNAIPAPTTT KSCRETFLKC FCRFINKGVF
WASPILSSVW GWGARLGHRA AGAGLCSGCA GHCLSHCLGC
LSVPPKELRA AGHLSSPGYL PSYERVPHLP HPWALCAP.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 9 amino acid sequence:

(SEQ ID NO: 580)
MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDNRPALGS TAPPVHNVTS ASGSASGSAS TLVHNGTSAR
ATTTPASKST PFSIPSHHSD TPTTLASHST KTDASSTHHS
TVPPLTSSNH STSPQLSTGV SFFFLSFHIS NLQFNSSLED
PSTDYYQELQ RDISEMFLQI YKQGGFLGLS NIKFRPGSVV
VQLTLAFREG TINVHDVETQ FNQYKTEAAS RYNLTISDVS
VSDVPFPFSA QSGAGVPGWG IALLVLVCVL VALAIVYLIA
LAVCQCRRKN YGQLDIFPAR DTYHPMSEYP TYHTHGRYVP
PSSTDRSPYE KVSAGNGGSS LSYTNPAVAA TSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 11 amino acid sequence:

(SEQ ID NO: 581)
MTPGTQSPFF LLLLLTVLTA TTAPKPATVV TGSGHASSTP
GGEKETSATQ RSSVPSSTEK NALSTGVSFF FLSFHISNLQ
FNSSLEDPST DYYQELQRDI SEMFLQIYKQ GGFLGLSNIK
FRPGSVVVQL TLAFREGTIN VHDVETQFNQ YKTEAASRYN
LTISDVSVSD VPFPFSAQSG AGVPGWGIAL LVLVCVLVAL
AIVYLIALAV CQCRRKNYGQ LDIFPARDTY HPMSEYPTYH
THGRYVPPSS TDRSPYEKVS AGNGGSSLSY TNPAVAATSA
NL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 12 amino acid sequence:

(SEQ ID NO: 582)
MTPGTQSPFF LLLLLTVLTA TTAPKPATVV TGSGHASSTP
GGEKETSATQ RSSVPSSTEK NAFNSSLEDP STDYYQELQR
DISEMFLQIY KQGGFLGLSN IKFRPGSVVV QLTLAFREGT
INVHDVETQF NQYKTEAASR YNLTISDVSV WGWGARLGHR
AAGAGLCSGC AGHCLSHCLG CLSVPPKELR AAGHLSSPGY
LPSYERVPHL PHPWALCAP.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 13 amino acid sequence:

(SEQ ID NO: 583)
MTPGTQSPFF LLLLLTVLTA TTAPKPATVV TGSGHASSTP
GGEKETSATQ RSSVPSSTEK NAIYKQGGFL GLSNIKFRPG
SVVVQLTLAF REGTINVHDV ETQFNQYKTE AASRYNLTIS
DVSVSDVPFP FSAQSGAGVP GWGIALLVLV CVLVALAIVY
LIALAVCQCR RKNYGQLDIF PARDTYHPMS EYPTYHTHGR
YVPPSSTDRS PYEKVSAGNG GSSLSYTNPA VAATSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 14 amino acid sequence:

(SEQ ID NO: 584)
MTPGTQSPFF LLLLLTVLTG GEKETSATQR SSVPSSTEKN
AIYKQGGFLG LSNIKFRPGS VVVQLTLAFR EGTINVHDVE
TQFNQYKTEA ASRYNLTISD VSVSDVPFPF SAQSGAGVPG
WGIALLVLVC VLVALAIVYL IALAVCQCRR KNYGQLDIFP
ARDTYHPMSE YPTYHTHGRY VPPSSTDRSP YEKVSAGNGG
SSLSYTNPAV AATSANL.

In some cases, a suitable MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) of a MUC1 polypeptide comprising an amino acid sequence having at least 80%, at least 85%, at least 90%, at least 95%, at least 98%, at least 99%, or 100%, amino acid sequence identity to the following Homo sapiens MUC1 isoform 15 amino acid sequence:

(SEQ ID NO: 585)
MTPGTQSPFF LLLLLTVLTA TTAPKPATVV TGSGHASSTP
GGEKETSATQ RSSVPSSTEK NAFLQIYKQG GFLGLSNIKF
RPGSVVVQLT LAFREGTINV HDVETQFNQY KTEAASRYNL
TISDVSVSDV PFPFSAQSGA GVPGWGIALL VLVCVLVALA
IVYLIALAVC QCRRKNYGQL DIFPARDTYH PMSEYPTYHT
HGRYVPPSST DRSPYEKVSA GNGGSSLSYT NPAVAATSAN
L.

Non-limiting examples of suitable MUC1 peptides include: i) STAPPAHGV (SEQ ID NO:586); ii) STAPPVHNV (SEQ ID NO:587); iii) SLAPPVHNV (SEQ ID NO:588); iv) SLAPPAHGV (SEQ ID NO:589); v) SAPDTRPAP (SEQ ID NO:590); vi) VTSAPDTRPAPGSTAPPAHG (SEQ ID NO: 591); vii) PDTRPAPGSTAPPAHGVTSA (SEQ ID NO:592); and viii) LLLLTVLTV (SEQ ID NO:593).

In some cases, the MUC1 peptide present in a TMMP of the present disclosure presents an epitope specific to an HLA-A, -B, -C, -E, -F, or -G allele. In an embodiment, the MUC1 peptide present in a TMMP presents an epitope restricted to HLA-A*0101, A*0201, A*0301, A*1101, A*2301, A*2402, A*2407, A*3303, and/or A*3401. In an embodiment, the epitope peptide present in a TMMP presents an epitope restricted to HLA-B*0702, B*0801, B*1502, B*3802, B*4001, B*4601, and/or B*5301. In an embodiment, the epitope peptide present in a TMMP presents an epitope restricted to C*0102, C*0303, C*0304, C*0401, C*0602, C*0701, C*702, C*0801, and/or C*1502.

As one example, the MUC1 peptide STAPPAHGV (SEQ ID NO:586) presents an epitope when bound to an HLA complex comprising a β2M polypeptide and an A*1101 HLA-A heavy chain. As another example, the MUC1 peptide STAPPAHGV (SEQ ID NO:586) presents an epitope when bound to an HLA complex comprising a β2M polypeptide and an A*0201 HLA-A heavy chain. As another example, the MUC1 peptide STAPPVHNV (SEQ ID NO:587) presents an epitope when bound to an HLA complex comprising a β2M polypeptide and an A*0201 HLA-A heavy chain. As another example, the MUC1 peptide SLAPPVHNV (SEQ ID NO:588) presents an epitope when bound to an HLA complex comprising a β2M polypeptide and an A*0201 HLA-A heavy chain. As another example, the MUC1 peptide SLAPPAHGV (SEQ ID NO:589) presents an epitope when bound to an HLA complex comprising a β2M polypeptide and an A*0201 HLA-A heavy chain.

### HLA/Peptide Binding Assays

Whether a given peptide (e.g., a MUC1 peptide that comprises a MUC1 epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays.

### Cell-Based Binding Assay

As one example, a cell-based peptide-induced stabilization assay can be used to determine peptide-HLA class I binding. In this assay, a peptide of interest is allowed to bind to a TAP-deficient cell, i.e., a cell that has defective transporter associated with antigen processing (TAP) machinery, and consequently, few surface class I molecules. Such cells include, e.g., the human T2 cell line (T2 (174×CEM.T2; American Type Culture Collection (ATCC) No. CRL-1992). Henderson et al. (1992) Science 255:1264. Without efficient TAP-mediated transport of cytosolic peptides into the endoplasmic reticulum, assembled class I complexes are structurally unstable, and retained only transiently at the cell surface. However, when T2 cells are incubated with an exogenous peptide capable of binding class I, surface peptide-HLA class I complexes are stabilized and can be detected by flow cytometry with, e.g., a pan anti-class I monoclonal antibody. The stabilization and resultant increased life-span of peptide-HLA complexes on the cell surface by the addition of a peptide validates their identity. Analysis can be carried out using flow cytometry, e.g., where the pan-HLA class I antibody comprises a fluorescent label. Binding of the peptide to various allelic forms of HLA H chains can be tested by genetically modifying the T2 cells to express an allelic HLA H chain of interest.

The following is a non-limiting example of use of a T2 assay to assess peptide binding to HLA A*0201. T2 cells are washed in cell culture medium, and concentrated to 10⁶ cells/ml. Peptides of interest are prepared in cell culture medium and serially diluted providing concentrations of 200 μM, 100 μM, 20 μM and 2 μM. The cells are mixed 1:1 with each peptide dilution to give a final volume of 200 L and final peptide concentrations of 100 μM, 50 μM, 10 μM and 1 μM. A HLA A*0201 binding peptide, GILGFVFTL, and a non-HLA A*0201-restricted peptide, HPVGEADYF (HLA-B*3501), are included as positive and negative controls, respectively. The cell/peptide mixtures are kept at 37° C. 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

### Biochemical Binding Assay

HLA polypeptides (HLA heavy chain polypeptide complexed with β2M polypeptide) can be tested for binding to a peptide of interest in a cell-free in vitro assay system. For example, a labeled reference peptide (e.g., fluorescently labeled) is allowed to bind to HLA polypeptides (HLA heavy chain polypeptide complexed with β2M polypeptide), to form an HLA-reference peptide complex. The ability of a test peptide of interest to displace the labeled reference peptide from the HLA-reference peptide complex is tested. The relative binding affinity is calculated as the amount of test peptide needed to displace the bound reference peptide. See, e.g., van der Burg et al. (1995) Human Immunol. 44:189.

As another example, a peptide of interest can be incubated with an HLA molecule (HLA heavy chain complexed with a β2M polypeptide), and the stabilization of the HLA/peptide complex can be measured in an immunoassay format. The ability of a peptide of interest to stabilize an HLA molecule is compared to that of a control peptide presenting a known T-cell epitope. Detection of stabilization is based on the presence or absence of the native conformation of the HLA/peptide complex, detected using an anti-HLA antibody. See, e.g., Westrop et al. (2009) J. Immunol. Methods 341:76; Steinitz et al. (2012) Blood 119:4073; and U.S. Pat. No. 9,205,144.

### T-Cell Activation Assays

Whether a given peptide binds a class I HLA (comprising an HLA heavy chain and a β2M polypeptide), and, when bound to the HLA complex, can effectively present an epitope to a TCR, can be determined by assessing T-cell response to the peptide-HLA complex. T-cell responses that can be measured include, e.g., interferon-gamma (IFNγ) production, cytotoxic activity, and the like.

### ELISPOT Assay

Suitable assays include, e.g., an enzyme linked immunospot (ELISPOT) assay. In this assay, production of IFNγ by CD8⁺ T cells is measured following with an antigen-presenting cell (APC) that presents a peptide of interest complexed with HLA class I. Antibody to IFNγ is immobilized on wells of a multi-well plate. APCs are added to the wells, and incubated for a period of time with a peptide of interest, such that the peptide binds HLA class I on the surface of the APCs. CD8⁺ T cells specific for the peptide are added to the wells, and the plate is incubated for about 24 hours. The wells are then washed, and any IFNγ bound to the immobilized anti-IFNγ antibody is detected using a detectably labeled anti-IFNγ antibody. A colorimetric assay can be used. For example, the detectably labeled anti-IFNγ antibody can be a biotin-labeled anti-IFNγ antibody, which can be detected using, e.g., streptavidin conjugated to alkaline phosphatase. A BCIP/NBT (5-bromo-4-chloro-3-indolyl phosphate/nitro blue tetrazolium) solution is added, to develop the assay. The presence of IFNγ-secreting T cells is identified by colored spots. Negative controls include APCs not contacted with the peptide. APCs expressing various HLA H chain alleles can be used to determine whether a peptide of interest effectively binds to a HLA class I molecule comprising a particular HLA H chain.

### Cytotoxicity Assays

Whether a given peptide binds to a particular HLA class I H chain and, when bound to a HLA class I complex comprising the H chain, can effectively present an epitope to a TCR, can also be determined using a cytotoxicity assay. A cytotoxicity assay involves incubation of a target cell with a cytotoxic CD8⁺ T cell. The target cell displays on its surface a peptide/HLA class I complex comprising a peptide of interest and an HLA class I molecule comprising an HLA H chain to be tested. The target cells can be radioactively labeled, e.g., with ⁵¹Cr. Whether the target cell effectively presents an epitope to a TCR on the cytotoxic CD8⁺ T cell, thereby inducing cytotoxic activity by the CD8⁺ T cell toward the target cell, is determined by measuring release of ⁵¹Cr from the lysed target cell. Specific cytotoxicity can be calculated as the amount of cytotoxic activity in the presence of the peptide minus the amount of cytotoxic activity in the absence of the peptide.

Detection of Antigen-Specific T Cells with Peptide-HLA Tetramers

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Immunomodulatory Polypeptides

In some cases, an immunomodulatory polypeptide present in a TMMP of the present disclosure is a wild-type immunomodulatory polypeptide. In other cases, an immunomodulatory polypeptide present in a TMMP of the present disclosure is a variant immunomodulatory polypeptide that has reduced affinity for a co-immunomodulatory polypeptide, compared to the affinity of a corresponding wild-type immunomodulatory polypeptide for the co-immunomodulatory polypeptide. Suitable immunomodulatory domains that exhibit reduced affinity for a co-immunomodulatory domain can have from 1 amino acid (aa) to 20 aa differences from a wild-type immunomodulatory domain. For example, in some cases, a variant immunomodulatory polypeptide present in a TMMP of the present disclosure differs in amino acid sequence by 1 aa, 2 aa, 3 aa, 4 aa, 5 aa, 6 aa, 7 aa, 8 aa, 9 aa, or 10 aa, from a corresponding wild-type immunomodulatory polypeptide. As another example, in some cases, a variant immunomodulatory polypeptide present in a TMMP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type immunomodulatory polypeptide. As an example, in some cases, a variant immunomodulatory polypeptide present in a TMMP of the present disclosure includes 1, 2, 3, 4, 5, 6, 7, 8, 9, or 10 amino acid substitutions, compared to a corresponding reference (e.g., wild-type) immunomodulatory polypeptide. In some cases, variant immunomodulatory polypeptide present in a TMMP of the present disclosure includes a single amino acid substitution compared to a corresponding reference (e.g., wild-type) immunomodulatory polypeptide. In some cases, variant immunomodulatory polypeptide present in a TMMP of the present disclosure includes 2 amino acid substitutions (e.g., no more than 2 amino acid substitutions) compared to a corresponding reference (e.g., wild-type) immunomodulatory polypeptide. In some cases, variant immunomodulatory polypeptide present in a TMMP of the present disclosure includes 3 amino acid substitutions (e.g., no more than 3 amino acid substitutions) compared to a corresponding reference (e.g., wild-type) immunomodulatory polypeptide. In some cases, variant immunomodulatory polypeptide present in a TMMP of the present disclosure includes 4 amino acid substitutions (e.g., no more than 4 amino acid substitutions) compared to a corresponding reference (e.g., wild-type) immunomodulatory polypeptide. In some cases, variant immunomodulatory polypeptide present in a TMMP of the present disclosure includes 5 amino acid substitutions (e.g., no more than 5 amino acid substitutions) compared to a corresponding reference (e.g., wild-type) immunomodulatory polypeptide. In some cases, variant immunomodulatory polypeptide present in a TMMP of the present disclosure includes 6 amino acid substitutions (e.g., no more than 6 amino acid substitutions) compared to a corresponding reference (e.g., wild-type) immunomodulatory polypeptide. In some cases, variant immunomodulatory polypeptide present in a TMMP of the present disclosure includes 7 amino acid substitutions (e.g., no more than 7 amino acid substitutions) compared to a corresponding reference (e.g., wild-type) immunomodulatory polypeptide. In some cases, variant immunomodulatory polypeptide present in a TMMP of the present disclosure includes 8 amino acid substitutions (e.g., no more than 8 amino acid substitutions) compared to a corresponding reference (e.g., wild-type) immunomodulatory polypeptide. In some cases, variant immunomodulatory polypeptide present in a TMMP of the present disclosure includes 9 amino acid substitutions (e.g., no more than 9 amino acid substitutions) compared to a corresponding reference (e.g., wild-type) immunomodulatory polypeptide. In some cases, variant immunomodulatory polypeptide present in a TMMP of the present disclosure includes 10 amino acid substitutions (e.g., no more than 10 amino acid substitutions) compared to a corresponding reference (e.g., wild-type) immunomodulatory polypeptide.

In some cases, variant immunomodulatory polypeptide present in a TMMP of the present disclosure includes 11 amino acid substitutions (e.g., no more than 11 amino acid substitutions) compared to a corresponding reference (e.g., wild-type) immunomodulatory polypeptide.

In some cases, variant immunomodulatory polypeptide present in a TMMP of the present disclosure includes 12 amino acid substitutions (e.g., no more than 12 amino acid substitutions) compared to a corresponding reference (e.g., wild-type) immunomodulatory polypeptide.

In some cases, variant immunomodulatory polypeptide present in a TMMP of the present disclosure includes 13 amino acid substitutions (e.g., no more than 13 amino acid substitutions) compared to a corresponding reference (e.g., wild-type) immunomodulatory polypeptide.

In some cases, variant immunomodulatory polypeptide present in a TMMP of the present disclosure includes 14 amino acid substitutions (e.g., no more than 14 amino acid substitutions) compared to a corresponding reference (e.g., wild-type) immunomodulatory polypeptide.

In some cases, variant immunomodulatory polypeptide present in a TMMP of the present disclosure includes 15 amino acid substitutions (e.g., no more than 15 amino acid substitutions) compared to a corresponding reference (e.g., wild-type) immunomodulatory polypeptide.

In some cases, variant immunomodulatory polypeptide present in a TMMP of the present disclosure includes 16 amino acid substitutions (e.g., no more than 16 amino acid substitutions) compared to a corresponding reference (e.g., wild-type) immunomodulatory polypeptide.

In some cases, variant immunomodulatory polypeptide present in a TMMP of the present disclosure includes 17 amino acid substitutions (e.g., no more than 17 amino acid substitutions) compared to a corresponding reference (e.g., wild-type) immunomodulatory polypeptide.

In some cases, variant immunomodulatory polypeptide present in a TMMP of the present disclosure includes 18 amino acid substitutions (e.g., no more than 18 amino acid substitutions) compared to a corresponding reference (e.g., wild-type) immunomodulatory polypeptide.

In some cases, variant immunomodulatory polypeptide present in a TMMP of the present disclosure includes 19 amino acid substitutions (e.g., no more than 19 amino acid substitutions) compared to a corresponding reference (e.g., wild-type) immunomodulatory polypeptide.

In some cases, variant immunomodulatory polypeptide present in a TMMP of the present disclosure includes 20 amino acid substitutions (e.g., no more than 20 amino acid substitutions) compared to a corresponding reference (e.g., wild-type) immunomodulatory polypeptide.

As discussed above, a variant immunomodulatory polypeptide suitable for inclusion in a TMMP of the present disclosure exhibits reduced affinity for a cognate co-immunomodulatory polypeptide, compared to the affinity of a corresponding wild-type immunomodulatory polypeptide for the cognate co-immunomodulatory polypeptide.

Exemplary pairs of immunomodulatory polypeptide and cognate co-immunomodulatory polypeptide include, but are not limited to:

a) 4-1BBL (immunomodulatory polypeptide) and 4-1BB (cognate co-immunomodulatory polypeptide);

b) PD-L1 (immunomodulatory polypeptide) and PD1 (cognate co-immunomodulatory polypeptide);

c) IL-2 (immunomodulatory polypeptide) and IL-2 receptor (cognate co-immunomodulatory polypeptide);

d) CD80 (immunomodulatory polypeptide) and CD86 (cognate co-immunomodulatory polypeptide);

e) CD86 (immunomodulatory polypeptide) and CD28 (cognate co-immunomodulatory polypeptide);

f) OX40L (CD252) (immunomodulatory polypeptide) and OX40 (CD134) (cognate co-immunomodulatory polypeptide);

g) Fas ligand (immunomodulatory polypeptide) and Fas (cognate co-immunomodulatory polypeptide);

h) ICOS-L (immunomodulatory polypeptide) and ICOS (cognate co-immunomodulatory polypeptide);

i) ICAM (immunomodulatory polypeptide) and LFA-1 (cognate co-immunomodulatory polypeptide);

j) CD30L (immunomodulatory polypeptide) and CD30 (cognate co-immunomodulatory polypeptide);

k) CD40 (immunomodulatory polypeptide) and CD40L (cognate co-immunomodulatory polypeptide);

l) CD83 (immunomodulatory polypeptide) and CD83L (cognate co-immunomodulatory polypeptide);

m) HVEM (CD270) (immunomodulatory polypeptide) and CD160 (cognate co-immunomodulatory polypeptide);

n) JAG1 (CD339) (immunomodulatory polypeptide) and Notch (cognate co-immunomodulatory polypeptide);

o) JAG1 (immunomodulatory polypeptide) and CD46 (cognate co-immunomodulatory polypeptide);

p) CD80 (immunomodulatory polypeptide) and CTLA4 (cognate co-immunomodulatory polypeptide);

q) CD86 (immunomodulatory polypeptide) and CTLA4 (cognate co-immunomodulatory polypeptide); and

r) CD70 (immunomodulatory polypeptide) and CD27 (cognate co-immunomodulatory polypeptide).

In some cases, a variant immunomodulatory polypeptide present in a TMMP of the present disclosure has a binding affinity for a cognate co-immunomodulatory polypeptide that is from 100 nM to 100 μM. For example, in some cases, a variant immunomodulatory polypeptide present in a TMMP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about M to about 50 μM, from about 50 μM to about 75 μM, or from about 75 μM to about 100 μM.

A variant immunomodulatory polypeptide present in a TMMP of the present disclosure exhibits reduced affinity for a cognate co-immunomodulatory polypeptide. Similarly, a TMMP of the present disclosure that comprises a variant immunomodulatory polypeptide exhibits reduced affinity for a cognate co-immunomodulatory polypeptide. Thus, for example, a TMMP of the present disclosure that comprises a variant immunomodulatory polypeptide has a binding affinity for a cognate co-immunomodulatory polypeptide that is from 100 nM to 100 μM. For example, in some cases, a TMMP of the present disclosure that comprises a variant immunomodulatory polypeptide has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

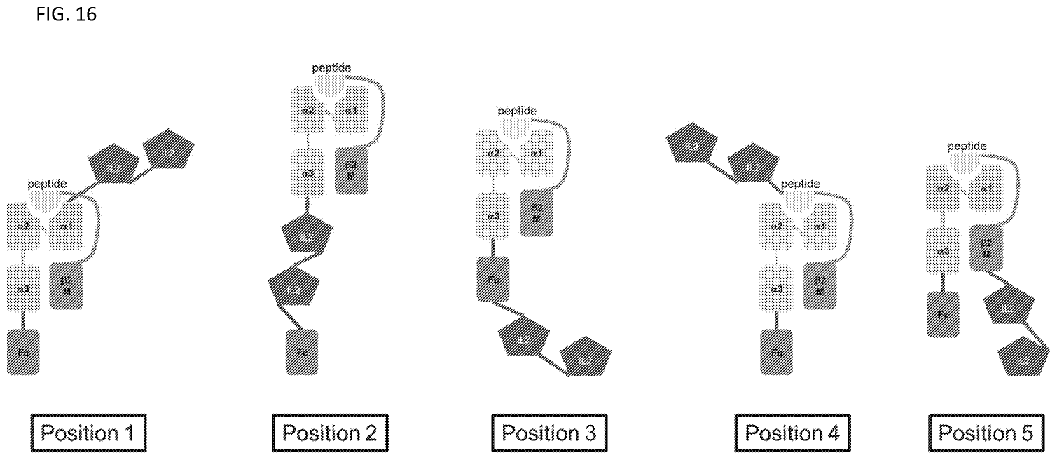

As depicted schematically in FIG. 16, an immunomodulatory polypeptide (i.e., one or more immunomodulatory polypeptides) can be present in a TMMP of the present disclosure at any of a variety of positions. FIG. 16 depicts the position of two copies of a variant IL-2 polypeptide; however, the immunomodulatory polypeptide can be any of a variety of immunomodulatory polypeptides, as described herein. As depicted in FIG. 16, an immunomodulatory polypeptide can be: 1) N-terminal to the MHC class I heavy chain; 2) C-terminal to the MHC class I heavy chain and N-terminal to the Ig Fc polypeptide; in other words, between the MHC class I heavy chain and the Ig Fc polypeptide; 3) C-terminal to the Ig Fc polypeptide; 4) N-terminal to the peptide epitope; or 5) C-terminal to the β2M polypeptide.

### PD-L1 Variants

As one non-limiting example, in some cases, a variant immunomodulatory polypeptide present in a TMMP of the present disclosure is a variant PD-L1 polypeptide. Wild-type PD-L1 binds to PD1.

A wild-type human PD-L1 polypeptide can comprise the following amino acid sequence:

(SEQ ID NO: 1)
MRIFAVFIFM TYWHLLNAFT VTVPKDLYVV EYGSNMTIEC
KFPVEKQLDL AALIVYWEME DKNIIQFVHG EEDLKVQHSS
YRQRARLLKD QLSLGNAALQ ITDVKLQDAG VYRCMISYGG
ADYKRITVKV NAPYNKINQR ILVVDPVTSE HELTCQAEGY
PKAEVIWTSS DHQVLSGKTT TTNSKREEKL FNVTSTLRIN
TTTNEIFYCT FRRLDPEENH TAELVIPGNI LNVSIKICLT
LSPST.

A wild-type human PD-L1 ectodomain can comprise the following amino acid sequence:

(SEQ ID NO: 2)
FT VTVPKDLYVV EYGSNMTIEC KFPVEKQLDL AALIVYWEME
DKNIIQFVHG EEDLKVQHSS YRQRARLLKD QLSLGNAALQ
ITDVKLQDAG VYRCMISYGG ADYKRITVKV NAPYNKINQR
ILVVDPVTSE HELTCQAEGY PKAEVIWTSS DHQVLSGKTT
TTNSKREEKL FNVTSTLRIN TTTNEIFYCT FRRLDPEENH
TAELVIPGNI LNVSIKI.

A wild-type PD-1 polypeptide can comprise the following amino acid sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:3). In some cases, where a TMMP of the present disclosure comprises a variant PD-L1 polypeptide, a “cognate co-immunomodulatory polypeptide” is a PD-1 polypeptide comprising the amino acid sequence of SEQ ID NO:3.

In some cases, a variant PD-L1 polypeptide exhibits reduced binding affinity to PD-1 (e.g., a PD-1 polypeptide comprising the amino acid sequence set forth in SEQ ID NO:3), compared to the binding affinity of a PD-L1 polypeptide comprising the amino acid sequence set forth in SEQ ID NO:1 or SEQ ID NO:2. For example, in some cases, a variant PD-L1 polypeptide of the present disclosure binds PD-1 (e.g., a PD-1 polypeptide comprising the amino acid sequence set forth in SEQ ID NO:3) with a binding affinity that is at least 10%, at least 15%, at least 20%, at least 25%, at least 30%, at least 35%, at least 40%, at least 45%, at least 50% less, at least 55% less, at least 60% less, at least 65% less, at least 70% less, at least 75% less, at least 80% less, at least 85% less, at least 90% less, at least 95% less, or more than 95% less, than the binding affinity of a PD-L1 polypeptide comprising the amino acid sequence set forth in SEQ ID NO:1 or SEQ ID NO:2.

In some cases, a variant PD-L1 polypeptide has a binding affinity to PD-1 that is from 1 nM to 1 mM. In some cases, a variant PD-L1 polypeptide of the present disclosure has a binding affinity to PD-1 that is from 100 nM to 100 μM. As another example, in some cases, a variant PD-L1 polypeptide has a binding affinity for PD1 (e.g., a PD1 polypeptide comprising the amino acid sequence set forth in SEQ ID NO:3) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant PD-L1 polypeptide has a single amino acid substitution compared to the PD-L1 amino acid sequence set forth in SEQ ID NO:1 or SEQ ID NO:2. In some cases, a variant PD-L1 polypeptide has from 2 to 10 amino acid substitutions compared to the PD-L1 amino acid sequence set forth in SEQ ID NO:1 or SEQ ID NO:2. In some cases, a variant PD-L1 polypeptide has 2 amino acid substitutions compared to the PD-L1 amino acid sequence set forth in SEQ ID NO:1 or SEQ ID NO:2. In some cases, a variant PD-L1 polypeptide has 3 amino acid substitutions compared to the PD-L1 amino acid sequence set forth in SEQ ID NO:1 or SEQ ID NO:2. In some cases, a variant PD-L1 polypeptide has 4 amino acid substitutions compared to the PD-L1 amino acid sequence set forth in SEQ ID NO:1 or SEQ ID NO:2. In some cases, a variant PD-L1 polypeptide has 5 amino acid substitutions compared to the PD-L1 amino acid sequence set forth in SEQ ID NO:1 or SEQ ID NO:2. In some cases, a variant PD-L1 polypeptide has 6 amino acid substitutions compared to the PD-L1 amino acid sequence set forth in SEQ ID NO:1 or SEQ ID NO:2. In some cases, a variant PD-L1 polypeptide has 7 amino acid substitutions compared to the PD-L1 amino acid sequence set forth in SEQ ID NO:1 or SEQ ID NO:2. In some cases, a variant PD-L1 polypeptide has 8 amino acid substitutions compared to the PD-L1 amino acid sequence set forth in SEQ ID NO:1 or SEQ ID NO:2. In some cases, a variant PD-L1 polypeptide has 9 amino acid substitutions compared to the PD-L1 amino acid sequence set forth in SEQ ID NO:1 or SEQ ID NO:2. In some cases, a variant PD-L1 polypeptide has 10 amino acid substitutions compared to the PD-L1 amino acid sequence set forth in SEQ ID NO:1 or SEQ ID NO:2.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKXLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:398), where X is any amino acid other than Asp. In some cases, X is Ala. In some cases, X is Arg.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKDLYVV EYGSNMTIEC KFPVEKQLDL AALXVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:399), where X is any amino acid other than Ile. In some cases, X is Asp.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKDLYVV EYGSNMTIEC KFPVEKQLDL AALIVYWEME DKNIIQFVHG EXDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:400), where X is any amino acid other than Glu. In some cases, X is Arg.

### CD80 Variants

In some cases, a variant immunomodulatory polypeptide present in a TMMP of the present disclosure is a variant CD80 polypeptide. Wild-type CD80 binds to CD28. Wild-type CD80 also binds to CD86.

A wild-type amino acid sequence of the ectodomain of human CD80 can be as follows:

(SEQ ID NO: 4)
VIHVTK EVKEVATLSC GHNVSVEELA QTRIYWQKEK
KMVLTMMSGD MNIWPEYKNR TIFDITNNLS IVILALRPSD
EGTYECVVLK YEKDAFKREH LAEVTLSVKA DFPTPSISDF
EIPTSNIRRI ICSTSGGFPE PHLSWLENGE ELNAINTTVS
QDPETELYAV SSKLDFNMTT NHSFMCLIKY GHLRVNQTFN
WNTTKQEHFP DN.

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:5). In some cases, where a TMMP of the present disclosure comprises a variant CD80 polypeptide, a “cognate co-immunomodulatory polypeptide” is a CD28 polypeptide comprising the amino acid sequence of SEQ ID NO:5.

A wild-type CD28 amino acid sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:6)

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:7).

In some cases, a variant CD80 polypeptide exhibits reduced binding affinity to CD28, compared to the binding affinity of a CD80 polypeptide comprising the amino acid sequence set forth in SEQ ID NO:4 for CD28. For example, in some cases, a variant CD80 polypeptide binds CD28 with a binding affinity that is at least 10%, at least 15%, at least 20%, at least 25%, at least 30%, at least 35%, at least 40%, at least 45%, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD80 polypeptide comprising the amino acid sequence set forth in SEQ ID NO:4 for CD28 (e.g., a CD28 polypeptide comprising the amino acid sequence set forth in one of SEQ ID NO:5, 6, or 7).

In some cases, a variant CD80 polypeptide has a binding affinity to CD28 that is from 100 nM to 100 μM. As another example, in some cases, a variant CD80 polypeptide of the present disclosure has a binding affinity for CD28 (e.g., a CD28 polypeptide comprising the amino acid sequence set forth in SEQ ID NO:5, SEQ ID NO:6, or SEQ ID NO:7) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD80 polypeptide has a single amino acid substitution compared to the CD80 amino acid sequence set forth in SEQ ID NO:4. In some cases, a variant CD80 polypeptide has from 2 to 10 amino acid substitutions compared to the CD80 amino acid sequence set forth in SEQ ID NO:4. In some cases, a variant CD80 polypeptide has 2 amino acid substitutions compared to the CD80 amino acid sequence set forth in SEQ ID NO:4. In some cases, a variant CD80 polypeptide has 3 amino acid substitutions compared to the CD80 amino acid sequence set forth in SEQ ID NO:4. In some cases, a variant CD80 polypeptide has 4 amino acid substitutions compared to the CD80 amino acid sequence set forth in SEQ ID NO:4. In some cases, a variant CD80 polypeptide has 5 amino acid substitutions compared to the CD80 amino acid sequence set forth in SEQ ID NO:4. In some cases, a variant CD80 polypeptide has 6 amino acid substitutions compared to the CD80 amino acid sequence set forth in SEQ ID NO:4. In some cases, a variant CD80 polypeptide has 7 amino acid substitutions compared to the CD80 amino acid sequence set forth in SEQ ID NO:4. In some cases, a variant CD80 polypeptide has 8 amino acid substitutions compared to the CD80 amino acid sequence set forth in SEQ ID NO:4. In some cases, a variant CD80 polypeptide has 9 amino acid substitutions compared to the CD80 amino acid sequence set forth in SEQ ID NO:4. In some cases, a variant CD80 polypeptide has 10 amino acid substitutions compared to the CD80 amino acid sequence set forth in SEQ ID NO:4.

Suitable CD80 variants include a polypeptide that comprises an amino acid sequence having at least 90%, at least 95%, at least 98%, at least 99%, or 100%, amino acid sequence identity to any one of the following amino acid sequences:

VIHVTK EVKEVATLSC GHXVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:401), where X is any amino acid other than Asn. In some cases, X is Ala;

VIHVTK EVKEVATLSC GHNVSVEELA QTRIYWQKEK KMVLTMMSGD MNIWPEYKNR TIFDITXNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:402), where X is any amino acid other than Asn. In some cases, X is Ala;

VIHVTK EVKEVATLSC GHNVSVEELA QTRIYWQKEK KMVLTMMSGD MNIWPEYKNR TIFDITNNLS XVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:403), where X is any amino acid other than Ile. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLX YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:404), where X is any amino acid other than Lys. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS XDPETELYAV SSKLDFNMTT NHSFMCLIKY GHLRVNQTFN WNTTKQEHFP DN (SEQ ID NO:405), where X is any amino acid other than Gln. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QXPETELYAV SSKLDFNMTT NHSFMCLIKY GHLRVNQTFN WNTTKQEHFP DN (SEQ ID NO:406), where X is any amino acid other than Asp. In some cases, X is Ala;

VIHVTK EVKEVATLSC GHNVSVEEXA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:407), where X is any amino acid other than Leu. In some cases, X is Ala;

VIHVTK EVKEVATLSC GHNVSVEELA QTRIXWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:408), where X is any amino acid other than Tyr. In some cases, X is Ala;

VIHVTK EVKEVATLSC GHNVSVEELA QTRIYWXKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:409), where X is any amino acid other than Gln. In some cases, X is Ala;

VIHVTK EVKEVATLSC GHNVSVEELA QTRIYWQKEK KXVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:410), where X is any amino acid other than Met. In some cases, X is Ala;

VIHVTK EVKEVATLSC GHNVSVEELA QTRIYWQKEK KMXLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:411), where X is any amino acid other than Val. In some cases, X is Ala;

VIHVTK EVKEVATLSC GHNVSVEELA QTRIYWQKEK KMVLTMMSGD MNXWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:412), where X is any amino acid other than Ile. In some cases, X is Ala;

VIHVTK EVKEVATLSC GHNVSVEELA QTRIYWQKEK KMVLTMMSGD MNIWPEXKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:413), where X is any amino acid other than Tyr. In some cases, X is Ala;

VIHVTK EVKEVATLSC GHNVSVEELA QTRIYWQKEK KMVLTMMSGD MNIWPEYKNR TIFXITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:414), where X is any amino acid other than Asp. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DXPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:415), where X is any amino acid other than Phe. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVX QDPETELYAV SSKLDFNMTT NHSFMCLIKY GHLRVNQTFN WNTTKQEHFP DN (SEQ ID NO:416), where X is any amino acid other than Ser. In some cases, X is Ala; and

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTXSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:417), where X is any amino acid other than Pro. In some cases, X is Ala.

### CD86 Variants

In some cases, a variant immunomodulatory polypeptide present in a TMMP of the present disclosure is a variant CD86 polypeptide. Wild-type CD86 binds to CD28. In some cases, where a TMMP of the present disclosure comprises a variant CD86 polypeptide, a “cognate co-immunomodulatory polypeptide” is a CD28 polypeptide comprising the amino acid sequence of SEQ ID NO:5.

The amino acid sequence of the full ectodomain of a wild-type human CD86 can be as

(SEQ ID NO: 8)
APLKIQAYFNETADLPCQFANSQNQSLSELVVFWQDQENLVLNEVYLG
KEKFDSVHSKYMNRTSFDSDSWTLRLHNLQIKDKGLYQCIIHHKKPTG
MIRIHQMNSELSVLANFSQPEIVPISNITENVYINLTCSSIHGYPEPK
KMSVLLRTKNSTIEYDGIMQKSQDNVTELYDVSISLSVSFPDVTSNMT
IFCILETDKTRLLSSPFSIELEDPQPPPDHIP.

The amino acid sequence of the IgV domain of a wild-type human CD86 can be as follows:

(SEQ ID NO: 9)
APLKIQAYFNETADLPCQFANSQNQSLSELVVFWQDQENLVLNEVYLG
KEKFDSVHSKYMNRTSFDSDSWTLRLHNLQIKDKGLYQCIIHHKKPTG
MIRIHQMNSELSVL.

In some cases, a variant CD86 polypeptide exhibits reduced binding affinity to CD28, compared to the binding affinity of a CD86 polypeptide comprising the amino acid sequence set forth in SEQ ID NO:8 or SEQ ID NO:9 for CD28. For example, in some cases, a variant CD86 polypeptide binds CD28 with a binding affinity that is at least 10%, at least 15%, at least 20%, at least 25%, at least 30%, at least 35%, at least 40%, at least 45%, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD86 polypeptide comprising the amino acid sequence set forth in SEQ ID NO:8 or SEQ ID NO:9 for CD28 (e.g., a CD28 polypeptide comprising the amino acid sequence set forth in one of SEQ ID NO:5, 6, or 7).

In some cases, a variant CD86 polypeptide has a binding affinity to CD28 that is from 100 nM to 100 μM. As another example, in some cases, a variant CD86 polypeptide of the present disclosure has a binding affinity for CD28 (e.g., a CD28 polypeptide comprising the amino acid sequence set forth in one of SEQ ID NOs:5, 6, or 7) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD86 polypeptide has a single amino acid substitution compared to the CD86 amino acid sequence set forth in SEQ ID NO:8. In some cases, a variant CD86 polypeptide has from 2 to 10 amino acid substitutions compared to the CD86 amino acid sequence set forth in SEQ ID NO:8. In some cases, a variant CD86 polypeptide has 2 amino acid substitutions compared to the CD86 amino acid sequence set forth in SEQ ID NO:8. In some cases, a variant CD86 polypeptide has 3 amino acid substitutions compared to the CD86 amino acid sequence set forth in SEQ ID NO:8. In some cases, a variant CD86 polypeptide has 4 amino acid substitutions compared to the CD86 amino acid sequence set forth in SEQ ID NO:8. In some cases, a variant CD86 polypeptide has 5 amino acid substitutions compared to the CD86 amino acid sequence set forth in SEQ ID NO:8. In some cases, a variant CD86 polypeptide has 6 amino acid substitutions compared to the CD86 amino acid sequence set forth in SEQ ID NO:8. In some cases, a variant CD86 polypeptide has 7 amino acid substitutions compared to the CD86 amino acid sequence set forth in SEQ ID NO:8. In some cases, a variant CD86 polypeptide has 8 amino acid substitutions compared to the CD86 amino acid sequence set forth in SEQ ID NO:8. In some cases, a variant CD86 polypeptide has 9 amino acid substitutions compared to the CD86 amino acid sequence set forth in SEQ ID NO:8. In some cases, a variant CD86 polypeptide has 10 amino acid substitutions compared to the CD86 amino acid sequence set forth in SEQ ID NO:8.

In some cases, a variant CD86 polypeptide has a single amino acid substitution compared to the CD86 amino acid sequence set forth in SEQ ID NO:9. In some cases, a variant CD86 polypeptide has from 2 to 10 amino acid substitutions compared to the CD86 amino acid sequence set forth in SEQ ID NO:9. In some cases, a variant CD86 polypeptide has 2 amino acid substitutions compared to the CD86 amino acid sequence set forth in SEQ ID NO:9. In some cases, a variant CD86 polypeptide has 3 amino acid substitutions compared to the CD86 amino acid sequence set forth in SEQ ID NO:9. In some cases, a variant CD86 polypeptide has 4 amino acid substitutions compared to the CD86 amino acid sequence set forth in SEQ ID NO:9. In some cases, a variant CD86 polypeptide has 5 amino acid substitutions compared to the CD86 amino acid sequence set forth in SEQ ID NO:9. In some cases, a variant CD86 polypeptide has 6 amino acid substitutions compared to the CD86 amino acid sequence set forth in SEQ ID NO:9. In some cases, a variant CD86 polypeptide has 7 amino acid substitutions compared to the CD86 amino acid sequence set forth in SEQ ID NO:9. In some cases, a variant CD86 polypeptide has 8 amino acid substitutions compared to the CD86 amino acid sequence set forth in SEQ ID NO:9. In some cases, a variant CD86 polypeptide has 9 amino acid substitutions compared to the CD86 amino acid sequence set forth in SEQ ID NO:9. In some cases, a variant CD86 polypeptide has 10 amino acid substitutions compared to the CD86 amino acid sequence set forth in SEQ ID NO:9.

Suitable CD86 variants include a polypeptide that comprises an amino acid sequence having at least 90%, at least 95%, at least 98%, at least 99%, or 100%, amino acid sequence identity to any one of the following amino acid sequences:

APLKIQAYFNETADLPCQFANSQNQSLSELVVFWQDQENLVLNEVYLGKEKFDSV HSKYMXRTSFDSDSWTLRLHNLQIKDKGLYQCIIHHKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:418), where X is any amino acid other than Asn. In some cases, X is Ala;

APLKIQAYFNETADLPCQFANSQNQSLSELVVFWQDQENLVLNEVYLGKEKFDSV HSKYMNRTSFXSDSWTLRLHNLQIKDKGLYQCIIHHKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:419), where X is any amino acid other than Asp. In some cases, X is Ala;

APLKIQAYFNETADLPCQFANSQNQSLSELVVFWQDQENLVLNEVYLGKEKFDSV HSKYMNRTSFDSDSXTLRLHNLQIKDKGLYQCIIHHKKPTGMIRIHQMNSELSVLANFSQPEIVPI SNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:420), where X is any amino acid other than Trp. In some cases, X is Ala;

APLKIQAYFNETADLPCQFANSQNQSLSELVVFWQDQENLVLNEVYLGKEKFDSV HSKYMNRTSFDSDSWTLRLHNLQIKDKGLYQCIIHXKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:421), where X is any amino acid other than His. In some cases, X is Ala;

APLKIQAYFNETADLPCQFANSQNQSLSELVVFWQDQENLVLNEVYLGKEKFDSV HSKYMXRTSFDSDSWTLRLHNLQIKDKGLYQCIIHHKKPTGMIRIHQMNSELSVL (SEQ ID NO:422), where X is any amino acid other than Asn. In some cases, X is Ala;

APLKIQAYFNETADLPCQFANSQNQSLSELVVFWQDQENLVLNEVYLGKEKFDSV HSKYMNRTSFXSDSWTLRLHNLQIKDKGLYQCIIHHKKPTGMIRIHQMNSELSVL (SEQ ID NO:423), where X is any amino acid other than Asp. In some cases, X is Ala;

APLKIQAYFNETADLPCQFANSQNQSLSELVVFWQDQENLVLNEVYLGKEKFDSV HSKYMNRTSFDSDSXTLRLHNLQIKDKGLYQCIIHHKKPTGMIRIHQMNSELSVL (SEQ ID NO:424), where X is any amino acid other than Trp. In some cases, X is Ala;

APLKIQAYFNETADLPCQFANSQNQSLSELVVFWQDQENLVLNEVYLGKEKFDSV HSKYMNRTSFDSDSWTLRLHNLQIKDKGLYQCIIHXKKPTGMIRIHQMNSELSVL (SEQ ID NO:425), where X is any amino acid other than His. In some cases, X is Ala;

APLKIQAYFNETADLPCQFANSQNQSLSELVVFWQDQENLXLNEVYLGKEKFDSV HSKYMNRTSFDSDSWTLRLHNLQIKDKGLYQCIIHHKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:426), where X is any amino acid other than Val. In some cases, X is Ala;

APLKIQAYFNETADLPCQFANSQNQSLSELVVFWQDQENLXLNEVYLGKEKFDSV HSKYMNRTSFDSDSWTLRLHNLQIKDKGLYQCIIHHKKPTGMIRIHQMNSELSVL (SEQ ID NO:427), where X is any amino acid other than Val. In some cases, X is Ala;

APLKIQAYFNETADLPCQFANSQNQSLSELVVFWXDQENLVLNEVYLGKEKFDSV HSKYMNRTSFDSDSWTLRLHNLQIKDKGLYQCIIHHKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:428), where X is any amino acid other than Gln. In some cases, X is Ala;

APLKIQAYFNETADLPCQFANSQNQSLSELVVFWXDQENLVLNEVYLGKEKFDSV HSKYMNRTSFDSDSWTLRLHNLQIKDKGLYQCIIHHKKPTGMIRIHQMNSELSVL (SEQ ID NO:429), where X is any amino acid other than Gln. In some cases, X is Ala;

APLKIQAYFNETADLPCQFANSQNQSLSELVVXWQDQENLVLNEVYLGKEKFDS VHSKYMNRTSFDSDSWTLRLHNLQIKDKGLYQCIIHHKKPTGMIRIHQMNSELSVLANFSQPEIV PISNITENVYINLTCSSIHGYPEPKKMSVLLRTKNSTIEYDGIMQKSQDNVTELYDVSISLSVSFPD VTSNMTIFCILETDKTRLLSSPFSIELEDPQPPPDHIP (SEQ ID NO:430), where X is any amino acid other than Phe. In some cases, X is Ala;

APLKIQAYFNETADLPCQFANSQNQSLSELVVXWQDQENLVLNEVYLGKEKFDS VHSKYMNRTSFDSDSWTLRLHNLQIKDKGLYQCIIHHKKPTGMIRIHQMNSELSVL (SEQ ID NO:431), where X is any amino acid other than Phe. In some cases, X is Ala;

APLKIQAYFNETADLPCQFANSQNQSLSELVVFWQDQENLVLNEVYLGKEKFDSV HSKYMNRTSFDSDSWTXRLHNLQIKDKGLYQCIIHHKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:432), where X is any amino acid other than Leu. In some cases, X is Ala;

APLKIQAYFNETADLPCQFANSQNQSLSELVVFWQDQENLVLNEVYLGKEKFDSV HSKYMNRTSFDSDSWTXRLHNLQIKDKGLYQCIIHHKKPTGMIRIHQMNSELSVL (SEQ ID NO:433), where X is any amino acid other than Leu. In some cases, X is Ala;

APLKIQAYFNETADLPCQFANSQNQSLSELVVFWQDQENLVLNEVYLGKEKFDSV HSKXMNRTSFDSDSWTLRLHNLQIKDKGLYQCIIHHKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:434), where X is any amino acid other than Tyr. In some cases, X is Ala;

APLKIQAYFNETADLPCQFANSQNQSLSELVVFWQDQENLVLNEVYLGKEKFDSV HSKXMNRTSFDSDSWTLRLHNLQIKDKGLYQCIIHHKKPTGMIRIHQMNSELSVL (SEQ ID NO:435), where X is any amino acid other than Tyr. In some cases, X is Ala;

APLKIQAYFNETADLPCQFANSQNQSLSELVVFWQDQENLVLNEVYLGKEKFDSV HSKYMXRTSFDSDSWTLRLHNLQIKDKGLYQCIIHXKKPTGMIRIHQMNSELSVLANFSQPEIVP ISNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:436), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSV HSKYMXRTSFDSDSWTLRLHNLQIKDKGLYQCIIHXKKPTGMIRIHQMNSELSVL (SEQ ID NO:437), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSV HSKYMNRTSFX₁SDSWTLRLHNLQIKDKGLYQCIIHX₂KKPTGMIRIHQMNSELSVLANFSQPEIV PISNITENVYINLTCSSIHGYPEPKKMSVLLRTKNSTIEYDGIMQKSQDNVTELYDVSISLSVSFPD VTSNMTIFCILETDKTRLLSSPFSIELEDPQPPPDHIP (SEQ ID NO:438), where X₁ is any amino acid other than Asp, and X₂ is any amino acid other than His. In some cases, X₁ is Ala and X₂ is Ala;

APLKIQAYFNETADLPCQFANSQNQSLSELVVFWQDQENLVLNEVYLGKEKFDSV HSKYMNRTSFX₁SDSWTLRLHNLQIKDKGLYQCIIHX₂KKPTGMIRIHQMNSELSVL (SEQ ID NO:439), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSV HSKYMX₁RTSFX₂SDSWTLRLHNLQIKDKGLYQCIIHX₃KKPTGMIRIHQMNSELSVLANFSQPEI VPISNITENVYINLTCSSIHGYPEPKKMSVLLRTKNSTIEYDGIMQKSQDNVTELYDVSISLSVSFP DVTSNMTIFCILETDKTRLLSSPFSIELEDPQPPPDHIP (SEQ ID NO:440), where X1 is any amino acid other than Asn, X₂ is any amino acid other than Asp, and X₃ is any amino acid other than His. In some cases, X1 is Ala, X₂ is Ala, and X₃ is Ala; and

APLKIQAYFNETADLPCQFANSQNQSLSELVVFWQDQENLVLNEVYLGKEKFDSV HSKYMX₁RTSFX₂SDSWTLRLHNLQIKDKGLYQCIIHX₃KKPTGMIRIHQMNSELSVL (SEQ ID NO:441), where X₁ is any amino acid other than Asn, X₂ is any amino acid other than Asp, and X₃ is any amino acid other than His. In some cases, X₁ is Ala, X₂ is Ala, and X₃ is Ala.

### 4-1BBL Variants

In some cases, a variant immunomodulatory polypeptide present in a TMMP of the present disclosure is a variant 4-1BBL polypeptide. Wild-type 4-1BBL binds to 4-1BB (CD137).

A wild-type 4-1BBL amino acid sequence can be as follows:

(SEQ ID NO: 10)
MEYASDASLD PEAPWPPAPR ARACRVLPWA LVAGLLLLLL
LAAACAVFLA CPWAVSGARA SPGSAASPRL REGPELSPDD
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs:11-13, as follows:

(SEQ ID NO: 11)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 12)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 13)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:14). In some cases, where a TMMP of the present disclosure comprises a variant 4-1BBL polypeptide, a “cognate co-immunomodulatory polypeptide” is a 4-1BB polypeptide comprising the amino acid sequence of SEQ ID NO:14.

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs:10-13. For example, in some cases, a variant 4-1BBL polypeptide of the present disclosure binds 4-1BB with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in one of SEQ ID NOs:10-13 for a 4-1BB polypeptide (e.g., a 4-1BB polypeptide comprising the amino acid sequence set forth in SEQ ID NO:14), when assayed under the same conditions.

In some cases, a variant 4-1BBL polypeptide has a binding affinity to 4-1BB that is from 100 nM to 100 μM. As another example, in some cases, a variant 4-1BBL polypeptide has a binding affinity for 4-1BB (e.g., a 4-1BB polypeptide comprising the amino acid sequence set forth in SEQ ID NO:14) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about M, from about 25 μM to about 50 μM, from about 50 μM to about 75 μM, or from about 75 μM to about 100 μM.

In some cases, a variant 4-1BBL polypeptide has a single amino acid substitution compared to the 4-1BBL amino acid sequence set forth in one of SEQ ID NOs:10-13. In some cases, a variant 4-1BBL polypeptide has from 2 to 10 amino acid substitutions compared to the 4-1BBL amino acid sequence set forth in one of SEQ ID NOs:10-13. In some cases, a variant 4-1BBL polypeptide has 2 amino acid substitutions compared to the 4-1BBL amino acid sequence set forth in one of SEQ ID NOs:10-13. In some cases, a variant 4-1BBL polypeptide has 3 amino acid substitutions compared to the 4-1BBL amino acid sequence set forth in one of SEQ ID NOs:10-13. In some cases, a variant 4-1BBL polypeptide has 4 amino acid substitutions compared to the 4-1BBL amino acid sequence set forth in one of SEQ ID NOs:10-13. In some cases, a variant 4-1BBL polypeptide has 5 amino acid substitutions compared to the 4-1BBL amino acid sequence set forth in one of SEQ ID NOs:10-13. In some cases, a variant 4-1BBL polypeptide has 6 amino acid substitutions compared to the 4-1BBL amino acid sequence set forth in one of SEQ ID NOs:10-13. In some cases, a variant 4-1BBL polypeptide has 7 amino acid substitutions compared to the 4-1BBL amino acid sequence set forth in one of SEQ ID NOs:10-13. In some cases, a variant 4-1BBL polypeptide has 8 amino acid substitutions compared to the 4-1BBL amino acid sequence set forth in one of SEQ ID NOs:10-13. In some cases, a variant 4-1BBL polypeptide has 9 amino acid substitutions compared to the 4-1BBL amino acid sequence set forth in one of SEQ ID NOs:10-13. In some cases, a variant 4-1BBL polypeptide has 10 amino acid substitutions compared to the 4-1BBL amino acid sequence set forth in one of SEQ ID NOs:10-13.

Suitable 4-1BBL variants include a polypeptide that comprises an amino acid sequence having at least 90%, at least 95%, at least 98%, at least 99%, or 100%, amino acid sequence identity to any one of the following amino acid sequences:

PAGLLDLRQG MFAQLVAQNV LLIDGPLSWY SDPGLAGVSL TGGLSYXEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:442), where X is any amino acid other than Lys. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWXLTQ GATVLGLFRV TPEIPAGLPS PRSE (SEQ ID NO:443), where X is any amino acid other than Gln. In some cases, X is Ala;

PAGLLDLRQG XFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:444), where X is any amino acid other than Met. In some cases, X is Ala;

PAGLLDLRQG MXAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:445), where X is any amino acid other than Phe. In some cases, X is Ala;

PAGLLDLRQG MFAXLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:446), where X is any amino acid other than Gln. In some cases, X is Ala;

PAGLLDLRQG MFAQXVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:447), where X is any amino acid other than Leu. In some cases, X is Ala;

PAGLLDLRQG MFAQLXAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:448), where X is any amino acid other than Val. In some cases, X is Ala;

PAGLLDLRQG MFAQLVAXNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:449), where X is any amino acid other than Gln. In some cases, X is Ala;

PAGLLDLRQG MFAQLVAQXV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:450), where X is any amino acid other than Asn. In some cases, X is Ala;

PAGLLDLRQG MFAQLVAQNX LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:451), where X is any amino acid other than Val. In some cases, X is Ala;

PAGLLDLRQG MFAQLVAQNV XLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:452), where X is any amino acid other than Leu. In some cases, X is Ala;

PAGLLDLRQG MFAQLVAQNV LXIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:453), where X is any amino acid other than Leu. In some cases, X is Ala;

PAGLLDLRQG MFAQLVAQNV LLXDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:454), where X is any amino acid other than Ile. In some cases, X is Ala;

PAGLLDLRQG MFAQLVAQNV LLIXGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:455), where X is any amino acid other than Asp. In some cases, X is Ala;

PAGLLDLRQG MFAQLVAQNV LLIDXPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:456), where X is any amino acid other than Gly. In some cases, X is Ala;

PAGLLDLRQG MFAQLVAQNV LLIDGXLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:457), where X is any amino acid other than Pro. In some cases, X is Ala;

PAGLLDLRQG MFAQLVAQNV LLIDGPXSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:458), where X is any amino acid other than Leu. In some cases, X is Ala;

PAGLLDLRQG MFAQLVAQNV LLIDGPLXWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:459), where X is any amino acid other than Ser. In some cases, X is Ala;

PAGLLDLRQG MFAQLVAQNV LLIDGPLSXY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:460), where X is any amino acid other than Trp. In some cases, X is Ala;

PAGLLDLRQG MFAQLVAQNV LLIDGPLSWX SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:461), where X is any amino acid other than Tyr. In some cases, X is Ala;

PAGLLDLRQG MFAQLVAQNV LLIDGPLSWY XDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:462), where X is any amino acid other than Ser. In some cases, X is Ala;

PAGLLDLRQG MFAQLVAQNV LLIDGPLSWY SXPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:463), where X is any amino acid other than Asp. In some cases, X is Ala;

PAGLLDLRQG MFAQLVAQNV LLIDGPLSWY SDXGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:464), where X is any amino acid other than Pro. In some cases, X is Ala;

PAGLLDLRQG MFAQLVAQNV LLIDGPLSWY SDPXLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:465), where X is any amino acid other than Gly. In some cases, X is Ala;

PAGLLDLRQG MFAQLVAQNV LLIDGPLSWY SDPGXAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:466), where X is any amino acid other than Leu. In some cases, X is Ala;

PAGLLDLRQG MFAQLVAQNV LLIDGPLSWY SDPGLAXVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:467), where X is any amino acid other than Gly. In some cases, X is Ala;

PAGLLDLRQG MFAQLVAQNV LLIDGPLSWY SDPGLAGXSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:468), where X is any amino acid other than Val. In some cases, X is Ala;

PAGLLDLRQG MFAQLVAQNV LLIDGPLSWY SDPGLAGVXL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:469), where X is any amino acid other than Ser. In some cases, X is Ala;

PAGLLDLRQG MFAQLVAQNV LLIDGPLSWY SDPGLAGVSX TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:470), where X is any amino acid other than Leu. In some cases, X is Ala;

PAGLLDLRQG MFAQLVAQNV LLIDGPLSWY SDPGLAGVSL XGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:471), where X is any amino acid other than Thr. In some cases, X is Ala;

PAGLLDLRQG MFAQLVAQNV LLIDGPLSWY SDPGLAGVSL TXGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:472), where X is any amino acid other than Gly. In some cases, X is Ala;

PAGLLDLRQG MFAQLVAQNV LLIDGPLSWY SDPGLAGVSL TGXLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:473), where X is any amino acid other than Gly. In some cases, X is Ala;

PAGLLDLRQG MFAQLVAQNV LLIDGPLSWY SDPGLAGVSL TGGXSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:474), where X is any amino acid other than Leu. In some cases, X is Ala;

PAGLLDLRQG MFAQLVAQNV LLIDGPLSWY SDPGLAGVSL TGGLXYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:475), where X is any amino acid other than Ser. In some cases, X is Ala;

PAGLLDLRQG MFAQLVAQNV LLIDGPLSWY SDPGLAGVSL TGGLSXKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:476), where X is any amino acid other than Tyr. In some cases, X is Ala;

PAGLLDLRQG MFAQLVAQNV LLIDGPLSWY SDPGLAGVSL TGGLSYKXDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:477), where X is any amino acid other than Glu. In some cases, X is Ala;

PAGLLDLRQG MFAQLVAQNV LLIDGPLSWY SDPGLAGVSL TGGLSYKEXT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:478), where X is any amino acid other than Asp. In some cases, X is Ala;

PAGLLDLRQG MFAQLVAQNV LLIDGPLSWY SDPGLAGVSL TGGLSYKEDX KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:479), where X is any amino acid other than Thr. In some cases, X is Ala;

PAGLLDLRQG MFAQLVAQNV LLIDGPLSWY SDPGLAGVSL TGGLSYKEDT XELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:480), where X is any amino acid other than Lys. In some cases, X is Ala;

PAGLLDLRQG MFAQLVAQNV LLIDGPLSWY SDPGLAGVSL TGGLSYKEDT KXLVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:481), where X is any amino acid other than Glu. In some cases, X is Ala;

PAGLLDLRQG MFAQLVAQNV LLIDGPLSWY SDPGLAGVSL TGGLSYKEDT KELVVAKAGV YYVXFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:482), where X is any amino acid other than Phe. In some cases, X is Ala;

PAGLLDLRQG MFAQLVAQNV LLIDGPLSWY SDPGLAGVSL TGGLSYKEDT KELVVAKAGV YYVFXQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:483), where X is any amino acid other than Phe. In some cases, X is Ala;

PAGLLDLRQG MFAQLVAQNV LLIDGPLSWY SDPGLAGVSL TGGLSYKEDT KELVVAKAGV YYVFFXLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:484), where X is any amino acid other than Gln. In some cases, X is Ala;

PAGLLDLRQG MFAQLVAQNV LLIDGPLSWY SDPGLAGVSL TGGLSYKEDT KELVVAKAGV YYVFFQXELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:485), where X is any amino acid other than Leu. In some cases, X is Ala;

PAGLLDLRQG MFAQLVAQNV LLIDGPLSWY SDPGLAGVSL TGGLSYKEDT KELVVAKAGV YYVFFQLXLR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:486), where X is any amino acid other than Glu. In some cases, X is Ala;

PAGLLDLRQG MFAQLVAQNV LLIDGPLSWY SDPGLAGVSL TGGLSYKEDT KELVVAKAGV YYVFFQLEXR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:487), where X is any amino acid other than Leu. In some cases, X is Ala;

PAGLLDLRQG MFAQLVAQNV LLIDGPLSWY SDPGLAGVSL TGGLSYKEDT KELVVAKAGV YYVFFQLELX RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:488), where X is any amino acid other than Arg. In some cases, X is Ala;

PAGLLDLRQG MFAQLVAQNV LLIDGPLSWY SDPGLAGVSL TGGLSYKEDT KELVVAKAGV YYVFFQLELR XVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:489), where X is any amino acid other than Arg. In some cases, X is Ala;

PAGLLDLRQG MFAQLVAQNV LLIDGPLSWY SDPGLAGVSL TGGLSYKEDT KELVVAKAGV YYVFFQLELR RXVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:490), where X is any amino acid other than Val. In some cases, X is Ala;

PAGLLDLRQG MFAQLVAQNV LLIDGPLSWY SDPGLAGVSL TGGLSYKEDT KELVVAKAGV YYVFFQLELR RVXAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:491), where X is any amino acid other than Val. In some cases, X is Ala;

PAGLLDLRQG MFAQLVAQNV LLIDGPLSWY SDPGLAGVSL TGGLSYKEDT KELVVAKAGV YYVFFQLELR RVVAXEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:492), where X is any amino acid other than Gly. In some cases, X is Ala;

PAGLLDLRQG MFAQLVAQNV LLIDGPLSWY SDPGLAGVSL TGGLSYKEDT KELVVAKAGV YYVFFQLELR RVVAGXGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:493), where X is any amino acid other than Glu. In some cases, X is Ala;

PAGLLDLRQG MFAQLVAQNV LLIDGPLSWY SDPGLAGVSL TGGLSYKEDT KELVVAKAGV YYVFFQLELR RVVAGEXSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:494), where X is any amino acid other than Gly. In some cases, X is Ala;

PAGLLDLRQG MFAQLVAQNV LLIDGPLSWY SDPGLAGVSL TGGLSYKEDT KELVVAKAGV YYVFFQLELR RVVAGEGXGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:495), where X is any amino acid other than Ser. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVXLPPASS EARNSAFGFQ GRLLHLSAGQ RLGVHLHTEA RARHAWQLTQ GATVLGLFRV TPEIPAGLPS PRSE (SEQ ID NO:496), where X is any amino acid other than Asp. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDXPPASS EARNSAFGFQ GRLLHLSAGQ RLGVHLHTEA RARHAWQLTQ GATVLGLFRV TPEIPAGLPS PRSE (SEQ ID NO:497), where X is any amino acid other than Leu. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLXPASS EARNSAFGFQ GRLLHLSAGQ RLGVHLHTEA RARHAWQLTQ GATVLGLFRV TPEIPAGLPS PRSE (SEQ ID NO:498), where X is any amino acid other than Pro. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPAXS EARNSAFGFQ GRLLHLSAGQ RLGVHLHTEA RARHAWQLTQ GATVLGLFRV TPEIPAGLPS PRSE (SEQ ID NO:499), where X is any amino acid other than Ser. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASX EARNSAFGFQ GRLLHLSAGQ RLGVHLHTEA RARHAWQLTQ GATVLGLFRV TPEIPAGLPS PRSE (SEQ ID NO:500), where X is any amino acid other than Ser. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS XARNSAFGFQ GRLLHLSAGQ RLGVHLHTEA RARHAWQLTQ GATVLGLFRV TPEIPAGLPS PRSE (SEQ ID NO:501), where X is any amino acid other than Glu. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EAXNSAFGFQ GRLLHLSAGQ RLGVHLHTEA RARHAWQLTQ GATVLGLFRV TPEIPAGLPS PRSE (SEQ ID NO:502), where X is any amino acid other than Arg. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARXSAFGFQ GRLLHLSAGQ RLGVHLHTEA RARHAWQLTQ GATVLGLFRV TPEIPAGLPS PRSE (SEQ ID NO:503), where X is any amino acid other than Asn. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNXAFGFQ GRLLHLSAGQ RLGVHLHTEA RARHAWQLTQ GATVLGLFRV TPEIPAGLPS PRSE (SEQ ID NO:504), where X is any amino acid other than Ser. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAXGFQ GRLLHLSAGQ RLGVHLHTEA RARHAWQLTQ GATVLGLFRV TPEIPAGLPS PRSE (SEQ ID NO:505), where X is any amino acid other than Phe. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGX RLGVHLHTEA RARHAWQLTQ GATVLGLFRV TPEIPAGLPS PRSE (SEQ ID NO:506), where X is any amino acid other than Gln. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ XLGVHLHTEA RARHAWQLTQ GATVLGLFRV TPEIPAGLPS PRSE (SEQ ID NO:507), where X is any amino acid other than Arg. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RXGVHLHTEA RARHAWQLTQ GATVLGLFRV TPEIPAGLPS PRSE (SEQ ID NO:508), where X is any amino acid other than Leu. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLXVHLHTEA RARHAWQLTQ GATVLGLFRV TPEIPAGLPS PRSE (SEQ ID NO:509), where X is any amino acid other than Gly. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGXHLHTEA RARHAWQLTQ GATVLGLFRV TPEIPAGLPS PRSE (SEQ ID NO:510), where X is any amino acid other than Val. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVXLHTEA RARHAWQLTQ GATVLGLFRV TPEIPAGLPS PRSE (SEQ ID NO:511), where X is any amino acid other than His. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHXHTEA RARHAWQLTQ GATVLGLFRV TPEIPAGLPS PRSE (SEQ ID NO:512), where X is any amino acid other than Leu. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLXTEA RARHAWQLTQ GATVLGLFRV TPEIPAGLPS PRSE (SEQ ID NO:513), where X is any amino acid other than His. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHXEA RARHAWQLTQ GATVLGLFRV TPEIPAGLPS PRSE (SEQ ID NO:514), where X is any amino acid other than Thr. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTXA RARHAWQLTQ GATVLGLFRV TPEIPAGLPS PRSE (SEQ ID NO:515), where X is any amino acid other than Glu. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA XARHAWQLTQ GATVLGLFRV TPEIPAGLPS PRSE (SEQ ID NO:516), where X is any amino acid other than Arg. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RAXHAWQLTQ GATVLGLFRV TPEIPAGLPS PRSE (SEQ ID NO:517), where X is any amino acid other than Arg. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARXAWQLTQ GATVLGLFRV TPEIPAGLPS PRSE (SEQ ID NO:518), where X is any amino acid other than His. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAXQLTQ GATVLGLFRV TPEIPAGLPS PRSE (SEQ ID NO:519), where X is any amino acid other than Trp. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQXTQ GATVLGLFRV TPEIPAGLPS PRSE (SEQ ID NO:520), where X is any amino acid other than Leu. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLXQ GATVLGLFRV TPEIPAGLPS PRSE (SEQ ID NO:521), where X is any amino acid other than Thr. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTX GATVLGLFRV TPEIPAGLPS PRSE (SEQ ID NO:522), where X is any amino acid other than Gln. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ XATVLGLFRV TPEIPAGLPS PRSE (SEQ ID NO:523), where X is any amino acid other than Gly. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GAXVLGLFRV TPEIPAGLPS PRSE (SEQ ID NO:524), where X is any amino acid other than Thr. In some cases, X is Ala; and

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATXLGLFRV TPEIPAGLPS PRSE (SEQ ID NO:525), where X is any amino acid other than Val. In some cases, X is Ala.

### IL-2 Variants

In some cases, a variant immunomodulatory polypeptide present in a TMMP of the present disclosure is a variant IL-2 polypeptide. Wild-type IL-2 binds to IL-2 receptor (IL-2R), i.e., a heterotrimeric polypeptide comprising IL-2Rα, IL-2Rβ, and IL-2Rγ.

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCOSIIS TLT (SEQ ID NO:15).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 16)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 17)
VNG TSQFTCFYNS RANISCVWSQ DGALQDTSCQ VHAWPDRRRW
NQTCELLPVS QASWACNLIL GAPDSQKLTT VDIVTLRVLC
REGVRWRVMA IQDFKPFENL RLMAPISLQV VHVETHRCNI
SWEISQASHY FERHLEFEAR TLSPGHTWEE APLLTLKQKQ
EWICLETLTP DTQYEFQVRV KPLQGEFTTW SPWSQPLAFR
TKPAALGKDT IPWLGHLLVG LSGAFGFIIL VYLLINCRNT
GPWLKKVLKC NTPDPSKFFS QLSSEHGGDV QKWLSSPFPS
SSFSPGGLAP EISPLEVLER DKVTQLLLQQ DKVPEPASLS
SNHSLTSCFT NQGYFFFHLP DALEIEACQV YFTYDPYSEE
DPDEGVAGAP TGSSPQPLQP LSGEDDAYCT FPSRDDLLLF
SPSLLGGPSP PSTAPGGSGA GEERMPPSLQ ERVPRDWDPQ
PLGPPTPGVP DLVDFQPPPE LVLREAGEEV PDAGPREGVS
FPWSRPPGQG EFRALNARLP LNTDAYLSLQ ELQGQDPTHL
V.
Human IL-2Rγ:
(SEQ ID NO: 18)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a TMMP of the present disclosure comprises a variant IL-2 polypeptide, a “cognate co-immunomodulatory polypeptide” is an IL-2R comprising polypeptides comprising the amino acid sequences of SEQ ID NO:16, 17, and 18.

In some cases, a variant IL-2 polypeptide exhibits reduced binding affinity to IL-2R, compared to the binding affinity of a IL-2 polypeptide comprising the amino acid sequence set forth in SEQ ID NO:15. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO:15 for an IL-2R (e.g., an IL-2R comprising polypeptides comprising the amino acid sequence set forth in SEQ ID NOs:16-18), when assayed under the same conditions.

In some cases, a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising polypeptides comprising the amino acid sequence set forth in SEQ ID NOs:16-18) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant IL-2 polypeptide has a single amino acid substitution compared to the IL-2 amino acid sequence set forth in SEQ ID NO:15. In some cases, a variant IL-2 polypeptide has from 2 to 10 amino acid substitutions compared to the IL-2 amino acid sequence set forth in SEQ ID NO:15. In some cases, a variant IL-2 polypeptide has 2 amino acid substitutions compared to the IL-2 amino acid sequence set forth in SEQ ID NO:15. In some cases, a variant IL-2 polypeptide has 3 amino acid substitutions compared to the IL-2 amino acid sequence set forth in SEQ ID NO:15. In some cases, a variant IL-2 polypeptide has 4 amino acid substitutions compared to the IL-2 amino acid sequence set forth in SEQ ID NO:15. In some cases, a variant IL-2 polypeptide has 5 amino acid substitutions compared to the IL-2 amino acid sequence set forth in SEQ ID NO:15. In some cases, a variant IL-2 polypeptide has 6 amino acid substitutions compared to the IL-2 amino acid sequence set forth in SEQ ID NO:15. In some cases, a variant IL-2 polypeptide has 7 amino acid substitutions compared to the IL-2 amino acid sequence set forth in SEQ ID NO:15. In some cases, a variant IL-2 polypeptide has 8 amino acid substitutions compared to the IL-2 amino acid sequence set forth in SEQ ID NO:15. In some cases, a variant IL-2 polypeptide has 9 amino acid substitutions compared to the IL-2 amino acid sequence set forth in SEQ ID NO:15. In some cases, a variant IL-2 polypeptide has 10 amino acid substitutions compared to the IL-2 amino acid sequence set forth in SEQ ID NO:15.

Suitable IL-2 variants include a polypeptide that comprises an amino acid sequence having at least 90%, at least 95%, at least 98%, at least 99%, or 100%, amino acid sequence identity to any one of the following amino acid sequences:

APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:527), where X is any amino acid other than Phe. In some cases, X is Ala. In some cases, X is Met. In some cases, X is Pro. In some cases, X is Ser. In some cases, X is Thr. In some cases, X is Trp. In some cases, X is Tyr. In some cases, X is Val. In some cases, X is His;

APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:528), where X is any amino acid other than Asp. In some cases, X is Ala;

APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:529), where X is any amino acid other than Glu. In some cases, X is Ala.

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:530), where X is any amino acid other than His. In some cases, X is Ala. In some cases, X is Thr. In some cases, X is Asn. In some cases, X is Cys. In some cases, X is Gln. In some cases, X is Met. In some cases, X is Val. In some cases, X is Trp;

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:531), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. In some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;

APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:532), where X is any amino acid other than Tyr. In some cases, X is Ala;

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO:533), where X is any amino acid other than Gln. In some cases, X is Ala;

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:534), where X₁ is any amino acid other than His, and where X₂ is any amino acid other than Phe. In some cases, X₁ is Ala. In some cases, X₂ is Ala. In some cases, X₁ is Ala; and X₂ is Ala. In some cases, X₁ is Thr; and X₂ is Ala;

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:535), where X₁ is any amino acid other than Asp; and where X₂ is any amino acid other than Phe. In some cases, X1 is Ala. In some cases, X₂ is Ala. In some cases, X₁ is Ala; and X₂ is Ala;

APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:536), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:537), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:538), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLLX₁ LQMILNGINN YKNPKLTRML TX₂KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:539), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:540), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO:541), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO:542), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X1 is Ala. In some cases, X₂ is Ala. In some cases, X₃ is Ala. In some cases, X₄ is Ala. In some cases, X₅ is Ala. In some cases, X1 is Ala; X₂ is Ala; X₃ is Ala; X₄ is Ala; X₅ is Ala; and

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:543), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

### Additional Polypeptides

A polypeptide chain of a TMMP of the present disclosure can include one or more polypeptides in addition to those described above. Suitable additional polypeptides include epitope tags and affinity domains. The one or more additional polypeptide can be included at the N-terminus of a polypeptide chain of a TMMP, at the C-terminus of a polypeptide chain of a TMMP, or internally within a polypeptide chain of a TMMP.

### Epitope Tag

Suitable epitope tags include, but are not limited to, hemagglutinin (HA; e.g., YPYDVPDYA (SEQ ID NO:544); FLAG (e.g., DYKDDDDK (SEQ ID NO:545); c-myc (e.g., EQKLISEEDL; SEQ ID NO:546), and the like.

### Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO:547), His×6 (HHHHHH) (SEQ ID NO:548), C-myc (EQKLISEEDL) (SEQ ID NO:549), Flag (DYKDDDDK) (SEQ ID NO:550), StrepTag (WSHPQFEK) (SEQ ID NO:551), hemagglutinin, e.g., HA Tag (YPYDVPDYA) (SEQ ID NO:552), glutathione-S-transferase (GST), thioredoxin, cellulose binding domain, RYIRS (SEQ ID NO:553), Phe-His-His-Thr (SEQ ID NO:554), chitin binding domain, S-peptide, T7 peptide, SH2 domain, C-end RNA tag, WEAAAREACCRECCARA (SEQ ID NO:555), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

### Drug Conjugates

A polypeptide chain of a TMMP of the present disclosure can comprise a small molecule drug linked (e.g., covalently attached) to the polypeptide chain. For example, where a TMMP of the present disclosure comprises an Fc polypeptide, the Fc polypeptide can comprise a covalently linked small molecule drug. In some cases, the small molecule drug is a cancer chemotherapeutic agent, e.g., a cytotoxic agent. A polypeptide chain of a TMMP of the present disclosure can comprise a cytotoxic agent linked (e.g., covalently attached) to the polypeptide chain. For example, where a TMMP of the present disclosure comprises an Fc polypeptide, the Fc polypeptide can comprise a covalently linked cytotoxic agent. Cytotoxic agents include prodrugs.

A drug (e.g., a cancer chemotherapeutic agent) can be linked directly or indirectly to a polypeptide chain of a TMMP of the present disclosure. For example, where a TMMP of the present disclosure comprises an Fc polypeptide, a drug (e.g., a cancer chemotherapeutic agent) can be linked directly or indirectly to the Fc polypeptide. Direct linkage can involve linkage directly to an amino acid side chain. Indirect linkage can be linkage via a linker. A drug (e.g., a cancer chemotherapeutic agent) can be linked to a polypeptide chain (e.g., an Fc polypeptide) of a TMMP of the present disclosure via a thioether bond, an amide bond, a carbamate bond, a disulfide bond, or an ether bond.

Linkers include cleavable linkers and non-cleavable linkers. In some cases, the linker is a protease-cleavable linker. Suitable linkers include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups. Non-limiting example of suitable linkers are: i) N-succinimidyl-[(N-maleimidopropionamido)-tetraethyleneglycol]ester (NHS-PEG4-maleimide); ii) N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

A polypeptide (e.g., an Fc polypeptide) can be modified with crosslinking reagents such as succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate, as described in the literature, to introduce 1-10 reactive groups. The modified Fc polypeptide is then reacted with a thiol-containing cytotoxic agent to produce a conjugate.

For example, where a TMMP of the present disclosure comprises an Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a cytotoxic agent. (L), if present, links (A) to (C). In some cases, the polypeptide chain comprising the Fc polypeptide can comprise more than one cytotoxic agent (e.g., 2, 3, 4, or 5, or more than 5, cytotoxic agents).

Suitable drugs include, e.g., rapamycin. Suitable drugs include, e.g., retinoids, such as all-trans retinoic acid (ATRA); vitamin D3; a vitamin D3 analog; and the like. As noted above, in some cases, a drug is a cytotoxic agent. Cytotoxic agents are known in the art. A suitable cytotoxic agent can be any compound that results in the death of a cell, or induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatin and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin.

For example, in some cases, the cytotoxic agent is a compound that inhibits microtubule formation in eukaryotic cells. Such agents include, e.g., maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. Maytansinoid compounds include, e.g., N(2′)-deacetyl-N(2′)-(3-mercapto-1-oxopropyl)-maytansine (DM1); N(2′)-deacetyl-N(2′)-(4-mercapto-1-oxopentyl)-maytansine (DM3); and N(2′)-deacetyl-N2-(4-mercapto-4-methyl-1-oxopentyl)-maytansine (DM4). Benzodiazepines include, e.g., indolinobenzodiazepines and oxazolidinobenzodiazepines.

Cytotoxic agents include taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; maytansine or an analog or derivative thereof; an auristatin or a functional peptide analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; 6 mercaptopurine; 6 thioguanine; cytarabine; fludarabin; 5 fluorouracil; decarbazine; hydroxyurea; asparaginase; gemcitabine; cladribine; an alkylating agent; a platinum derivative; duocarmycin A; duocarmycin SA; rachelmycin (CC-1065) or an analog or derivative thereof; an antibiotic; pyrrolo[2,1-c][1,4]-benzodiazepines (PDB); diphtheria toxin; ricin toxin; cholera toxin; a Shiga-like toxin; LT toxin; C3 toxin; Shiga toxin; pertussis toxin; tetanus toxin; soybean Bowman-Birk protease inhibitor; Pseudomonas exotoxin; alorin; saporin; modeccin; gelanin; abrin A chain; modeccin A chain; alpha-sarcin; Aleurites fordii proteins; dianthin proteins; Phytolacca americana proteins; Momordica charantia inhibitor; curcin; crotin; Sapaonaria officinalis inhibitor; gelonin; mitogellin; restrictocin; phenomycin; enomycin toxins; ribonuclease (RNase); DNase I; Staphylococcal enterotoxin A; pokeweed antiviral protein; diphtherin toxin; and Pseudomonas endotoxin.

### Exemplary TMMPs

A TMMP of the present disclosure comprises at least one heterodimer comprising: a) a first polypeptide comprising: i) a MUC1 peptide; and ii) first MHC polypeptide; b) a second polypeptide comprising a second MHC polypeptide, and c) at least one immunomodulatory polypeptide, where the first and/or the second polypeptide comprises the immunomodulatory polypeptide. Thus, in some cases, a TMMP of the present disclosure comprises at least one heterodimer comprising: a) a first polypeptide comprising: i) a MUC1 peptide; ii) first MHC polypeptide; and iii) at least one immunomodulatory polypeptide; and b) a second polypeptide comprising a second MHC polypeptide. In other instances, a TMMP of the present disclosure comprises at least one heterodimer comprising: a) a first polypeptide comprising: i) a MUC1 peptide; and ii) first MHC polypeptide; and b) a second polypeptide comprising: i) a second MHC polypeptide; and ii) at least one immunomodulatory polypeptide. In some cases, a TMMP of the present disclosure comprises at least one heterodimer comprising: a) a first polypeptide comprising: i) a MUC1 peptide; ii) first MHC polypeptide; and iii) at least one immunomodulatory polypeptide; and b) a second polypeptide comprising: i) a second MHC polypeptide; and ii) at least one immunomodulatory polypeptide. In some cases, the at least one immunomodulatory polypeptide is a wild-type immunomodulatory polypeptide. In other cases, the at least one immunomodulatory polypeptide is a variant immunomodulatory polypeptide that exhibits reduced affinity for a co-immunomodulatory polypeptide, compared to the affinity of a corresponding wild-type immunomodulatory polypeptide for the co-immunomodulatory polypeptide. In some cases, a TMMP of the present disclosure comprises two immunomodulatory polypeptides, where the two immunomodulatory polypeptides have the same amino acid sequence. In some cases, the at least one immunomodulatory polypeptide is an IL-2 polypeptide, e.g., a variant IL-2 polypeptide as described herein. Suitable MUC1 peptides are described herein.

As noted above, and as depicted schematically in FIG. 16, an immunomodulatory polypeptide (i.e., one or more immunomodulatory polypeptides) can be present in a TMMP of the present disclosure at any of a variety of positions. FIG. 16 depicts the position of two copies of a variant IL-2 polypeptide; however, the immunomodulatory polypeptide can be any of a variety of immunomodulatory polypeptide, as described herein. As depicted in FIG. 16, an immunomodulatory polypeptide can be: 1) N-terminal to the MHC class I heavy chain (position 1); 2) C-terminal to the MHC class I heavy chain and N-terminal to the Ig Fc polypeptide; in other words, between the MHC class I heavy chain and the Ig Fc polypeptide (position 2); 3) C-terminal to the Ig Fc polypeptide (position 3); 4) N-terminal to the peptide epitope (position 4); or 5) C-terminal to the β2M polypeptide (position 5). “Position 1” refers to a position of the immunomodulatory polypeptide on the same polypeptide chain as the class I MHC heavy chain and N-terminal to the class I MHC heavy chain; e.g., where the TMMP comprises: a) a first polypeptide comprising, in order from N-terminus to C-terminus: i) a peptide epitope (e.g., a MUC1 peptide); and ii) a β2M polypeptide; and b) a second polypeptide comprising, in order from N-terminus to C-terminus: i) one or more immunomodulatory polypeptides; and ii) a class I MHC heavy chain polypeptide. “Position 2” refers to a position of the immunomodulatory polypeptide on the same polypeptide chain as the class I MHC heavy chain and C-terminal to the class I MHC heavy chain, but not at the C-terminus of the polypeptide chain; e.g., where the TMMP comprises: a) a first polypeptide comprising, in order from N-terminus to C-terminus: i) a peptide epitope (e.g., a MUC1 peptide); and ii) a β2M polypeptide; and b) a second polypeptide comprising, in order from N-terminus to C-terminus: i) a class I MHC heavy chain polypeptide; ii) one or more immunomodulatory polypeptides; and iii) an Ig Fc polypeptide. “Position 3” refers to a position of the immunomodulatory polypeptide on the same polypeptide chain as the class I MHC heavy chain and at the C-terminus of the polypeptide chain; e.g., where the TMMP comprises: a) a first polypeptide comprising, in order from N-terminus to C-terminus: i) a peptide epitope (e.g., a MUC1 peptide); and ii) a β2M polypeptide; and b) a second polypeptide comprising, in order from N-terminus to C-terminus: i) a class I MHC heavy chain polypeptide; ii) an Ig Fc polypeptide; and iii) one or more immunomodulatory polypeptides. “Position 4” refers to a position of the immunomodulatory polypeptide on the same polypeptide chain as the β2M polypeptide and N-terminal to the peptide epitope and the β2M polypeptide; e.g., where the TMMP comprises: a) a first polypeptide comprising, in order from N-terminus to C-terminus: i) one or more immunomodulatory polypeptides; ii) a peptide epitope (e.g., a MUC1 peptide); and iii) a β2M polypeptide; and b) a second polypeptide comprising a class I MHC heavy chain polypeptide (e.g., a second polypeptide comprising, in order from N-terminus to C-terminus: i) a class I MHC heavy chain polypeptide; and ii) an Ig Fc polypeptide. “Position 5” refers to a position of the immunomodulatory polypeptide on the same polypeptide chain as the β2M polypeptide and C-terminal to the β2M polypeptide (e.g., at the C-terminus of the polypeptide chain); e.g., where the TMMP comprises: a) a first polypeptide comprising, in order from N-terminus to C-terminus: i) a peptide epitope (e.g., a MUC1 peptide); ii) a β2M polypeptide; and iii) one or more immunomodulatory polypeptides; and b) a second polypeptide comprising a class I MHC heavy chain polypeptide (e.g., a second polypeptide comprising, in order from N-terminus to C-terminus: i) a class I MHC heavy chain polypeptide; and ii) an Ig Fc polypeptide.

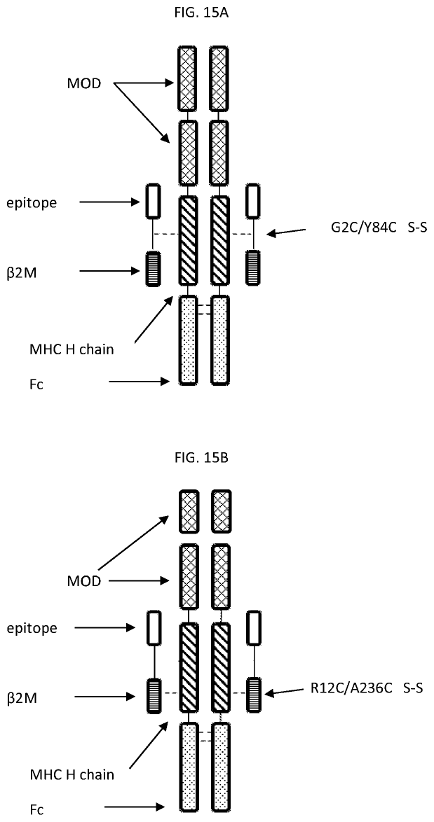

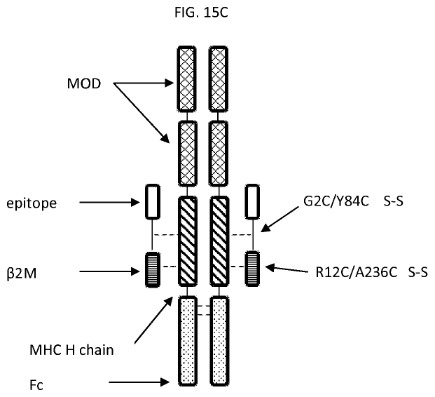

Furthermore, as discussed above and as depicted schematically in FIG. 15A-15C, the first polypeptide chain and the second polypeptide chain of a TMMP of the present disclosure can be linked by one or more disulfide bonds. For example, a TMMMP of the present disclosure can comprise: a) a first polypeptide chain comprising an β2M polypeptide having an R12C substitution; and b) a second polypeptide chain comprising a class I MHC heavy chain polypeptide having an A236C substitution; such that a disulfide bond forms between the Cys at position 12 of the β2M polypeptide in the first polypeptide chain and the Cys at position 236 of the class I MHC heavy chain polypeptide in the second polypeptide chain. As another example, a TMMMP of the present disclosure can comprise: a) a first polypeptide comprising, in order from N-terminus to C-terminus: i) a peptide epitope; ii) a peptide linker comprising a GCGGS(GGGGS)ₙ (SEQ ID NO:140) sequence, where n is 1, 2, or 3; and iii) a β2M polypeptide; and b) a second polypeptide comprising a class I MHC heavy chain polypeptide having a Y84C substitution, such that a disulfide bond forms between the Cys in the peptide linker in the first polypeptide chain and the Cys at position 84 of the class I MHC heavy chain polypeptide in the second polypeptide chain. In other examples, a TMMP of the present disclosure can comprise: a) a first polypeptide comprising, in order from N-terminus to C-terminus: i) a peptide epitope; ii) a peptide linker comprising a GCGGS(GGGGS)ₙ (SEQ ID NO:140) sequence, where n is 1, 2, or 3; and iii) a β2M polypeptide having an R12C substitution; and b) a second polypeptide comprising a class I MHC heavy chain polypeptide having a Y84C substitution and an A236C substitution; such that: i) a first disulfide bond forms between the Cys in the peptide linker in the first polypeptide chain and the Cys at position 84 of the class I MHC heavy chain polypeptide in the second polypeptide chain; and ii) a second disulfide bond forms between the Cys at position 12 of the β2M polypeptide in the first polypeptide chain and the Cys at position 236 of the class I MHC heavy chain polypeptide in the second polypeptide chain. For simplicity, the first disulfide bond is referred to as “G2C/Y84C”; and the second disulfide bond is referred to as “R12C/A236C.” A TMMP of the present disclosure can include: a) a G2C/Y84C disulfide bond and not an R12C/A236C disulfide bond; b) an R12C/A236C disulfide bond and not a G2C/Y84C disulfide bond; or c) a G2C/Y84C disulfide bond and an R12C/A236C disulfide bond.

A TMMP of the present disclosure can include: a) a G2C/Y84C disulfide bond and not an R12C/A236C disulfide bond; and b) at least one immunomodulatory polypeptide at position 1. A TMMP of the present disclosure can include: a) a G2C/Y84C disulfide bond and not an R12C/A236C disulfide bond; and b) at least one immunomodulatory polypeptide at position 2. A TMMP of the present disclosure can include: a) a G2C/Y84C disulfide bond and not an R12C/A236C disulfide bond; and b) at least one immunomodulatory polypeptide at position 3. A TMMP of the present disclosure can include: a) a G2C/Y84C disulfide bond and not an R12C/A236C disulfide bond; and b) at least one immunomodulatory polypeptide at position 4. A TMMP of the present disclosure can include: a) a G2C/Y84C disulfide bond and not an R12C/A236C disulfide bond; and b) at least one immunomodulatory polypeptide at position 5.

A TMMP of the present disclosure can include: a) an R12C/A236C disulfide bond and not a G2C/Y84C disulfide bond; and at least one immunomodulatory polypeptide at position 1. A TMMP of the present disclosure can include: a) an R12C/A236C disulfide bond and not a G2C/Y84C disulfide bond; and at least one immunomodulatory polypeptide at position 2. A TMMP of the present disclosure can include: a) an R12C/A236C disulfide bond and not a G2C/Y84C disulfide bond; and at least one immunomodulatory polypeptide at position 3. A TMMP of the present disclosure can include: a) an R12C/A236C disulfide bond and not a G2C/Y84C disulfide bond; and at least one immunomodulatory polypeptide at position 4. A TMMP of the present disclosure can include: a) an R12C/A236C disulfide bond and not a G2C/Y84C disulfide bond; and at least one immunomodulatory polypeptide at position 5.

A TMMP of the present disclosure can include: a) a G2C/Y84C disulfide bond and an R12C/A236C disulfide bond; and b) and at least one immunomodulatory polypeptide at position 1. A TMMP of the present disclosure can include: a) a G2C/Y84C disulfide bond and an R12C/A236C disulfide bond; and b) and at least one immunomodulatory polypeptide at position 2. A TMMP of the present disclosure can include: a) a G2C/Y84C disulfide bond and an R12C/A236C disulfide bond; and b) and at least one immunomodulatory polypeptide at position 3. A TMMP of the present disclosure can include: a) a G2C/Y84C disulfide bond and an R12C/A236C disulfide bond; and b) and at least one immunomodulatory polypeptide at position 4. A TMMP of the present disclosure can include: a) a G2C/Y84C disulfide bond and an R12C/A236C disulfide bond; and b) and at least one immunomodulatory polypeptide at position 5.

A TMMP of the present disclosure comprises at least one heterodimer comprising: a) a first polypeptide comprising: i) a MUC1 peptide; and ii) first MHC polypeptide; b) a second polypeptide comprising a second MHC polypeptide, and c) at least one immunomodulatory polypeptide, where the first and/or the second polypeptide comprises the immunomodulatory polypeptide. Thus, in some cases, a TMMP of the present disclosure comprises at least one heterodimer comprising: a) a first polypeptide comprising: i) a MUC1 peptide; ii) first MHC polypeptide; and iii) at least one immunomodulatory polypeptide; and b) a second polypeptide comprising a second MHC polypeptide. In other instances, a TMMP of the present disclosure comprises at least one heterodimer comprising: a) a first polypeptide comprising: i) a MUC1 peptide; and ii) first MHC polypeptide; and b) a second polypeptide comprising: i) a second MHC polypeptide; and ii) at least one immunomodulatory polypeptide. In some cases, a TMMP of the present disclosure comprises at least one heterodimer comprising: a) a first polypeptide comprising: i) a MUC1 peptide; ii) first MHC polypeptide; and iii) at least one immunomodulatory polypeptide; and b) a second polypeptide comprising: i) a second MHC polypeptide; and ii) at least one immunomodulatory polypeptide. In some cases, the at least one immunomodulatory polypeptide is a wild-type immunomodulatory polypeptide. In other cases, the at least one immunomodulatory polypeptide is a variant immunomodulatory polypeptide that exhibits reduced affinity for a co-immunomodulatory polypeptide, compared to the affinity of a corresponding wild-type immunomodulatory polypeptide for the co-immunomodulatory polypeptide. In some cases, a TMMP of the present disclosure comprises two immunomodulatory polypeptides, where the two immunomodulatory polypeptides have the same amino acid sequence. In some cases, the at least one immunomodulatory polypeptide is an IL-2 polypeptide, e.g., a variant IL-2 polypeptide as described herein. In some cases, the MUC1 peptide has the amino acid sequence STAPPVHNV (SEQ ID NO:587) and has a length of 9 amino acids. In some cases, the MUC1 peptide has the amino acid sequence SLAPPVHNV (SEQ ID NO:588) and has a length of 9 amino acids. In some cases, the MUC1 peptide has the amino acid sequence STAPPAHGV (SEQ ID NO:586) and has a length of 9 amino acids. In some cases, the MUC1 peptide has the amino acid sequence SLAPPAHGV (SEQ ID NO:589) and has a length of 9 amino acids. In some cases, the second MHC polypeptide is an HLA heavy chain that comprises an amino acid sequence having at least 95% amino acid sequence identity to an HLA-A*0201 polypeptide; and the first MHC polypeptide is a β2M polypeptide.

In some cases, a TMMP of the present disclosure comprises: a) a first polypeptide comprising, in order from N-terminus to C-terminus: i) a MUC1 peptide; ii) a first MHC polypeptide; and iii) at least one immunomodulatory polypeptide; and b) a second polypeptide comprising, in order from N-terminus to C-terminus: i) a second MHC polypeptide; and ii) an Ig Fc polypeptide. In some cases, the first MHC polypeptide is a β2M polypeptide; and the second MHC polypeptide is an HLA heavy chain polypeptide. In some cases, the HLA heavy chain polypeptide is an HLA-A*0201 polypeptide. In some cases, the HLA heavy chain polypeptide is an HLA-A*0201 polypeptide with an A236C substitution. In some cases, the first polypeptide comprises, in order from N-terminus to C-terminus: i) a peptide epitope; ii) a first MHC polypeptide; and iii) two immunomodulatory polypeptides, where the two immunomodulatory polypeptides have the same amino acid sequence. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, a peptide linker is between one or more of: i) the second MHC polypeptide and the Ig Fc polypeptide; ii) the epitope and the first MHC polypeptide; iii) the first MHC polypeptide and the immunomodulatory polypeptide; and (where the TMMP comprises two immunomodulatory polypeptides on the first polypeptide chain) iv) between the two immunomodulatory polypeptides. In some cases, the peptide linker comprises the amino acid sequence AAAGG (SEQ ID NO: 387). In some cases, the peptide linker comprises the amino acid sequence (GGGGS)n (SEQ ID NO: 254), where n is an integer from 1 to 10 (e.g., where n is 2, 3, or 4). In some cases, the MUC1 peptide has the amino acid sequence STAPPVHNV (SEQ ID NO:587) and has a length of 9 amino acids. In some cases, the MUC1 peptide has the amino acid sequence SLAPPVHNV (SEQ ID NO:588) and has a length of 9 amino acids. In some cases, the MUC1 peptide has the amino acid sequence STAPPAHGV (SEQ ID NO:586) and has a length of 9 amino acids. In some cases, the MUC1 peptide has the amino acid sequence SLAPPAHGV (SEQ ID NO:589) and has a length of 9 amino acids.

In some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) at least one immunomodulatory polypeptide; ii) a second MHC polypeptide; and iii) an Ig Fc polypeptide. In some cases, the first MHC polypeptide is a β2M polypeptide; and the second MHC polypeptide is an HLA heavy chain polypeptide. In some cases, the HLA heavy chain polypeptide is an HLA-A*0201 polypeptide. In some cases, the HLA heavy chain polypeptide is an HLA-A*0201 polypeptide with an A236C substitution. In some cases, the second polypeptide comprises, in order from N-terminus to C-terminus: i) two immunomodulatory polypeptides, where the two immunomodulatory polypeptides have the same amino acid sequence; ii) a second MHC polypeptide; and iii) an Ig Fc polypeptide. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, a peptide linker is between one or more of: i) the second MHC polypeptide and the Ig Fc polypeptide; ii) the epitope and the first MHC polypeptide; iii) the first MHC polypeptide and the immunomodulatory polypeptide; and (where the TMMP comprises two immunomodulatory polypeptides on the second polypeptide chain) iv) between the two immunomodulatory polypeptides. In some cases, the peptide linker comprises the amino acid sequence AAAGG (SEQ ID NO: 387). In some cases, the peptide linker comprises the amino acid sequence (GGGGS)n (SEQ ID NO: 254), where n is an integer from 1 to 10 (e.g., where n is 2, 3, or 4). In some cases, the MUC1 peptide has the amino acid sequence STAPPVHNV (SEQ ID NO:587) and has a length of 9 amino acids. In some cases, the MUC1 peptide has the amino acid sequence SLAPPVHNV (SEQ ID NO:588) and has a length of 9 amino acids. In some cases, the MUC1 peptide has the amino acid sequence STAPPAHGV (SEQ ID NO:589) and has a length of 9 amino acids. In some cases, the MUC1 peptide has the amino acid sequence SLAPPAHGV (SEQ ID NO:589) and has a length of 9 amino acids.

In some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) a second MHC polypeptide; ii) an Ig Fc polypeptide; and iii) at least one immunomodulatory polypeptide. In some cases, the first MHC polypeptide is a β2M polypeptide; and the second MHC polypeptide is an HLA heavy chain polypeptide. In some cases, the HLA heavy chain polypeptide is an HLA-A*0201 polypeptide. In some cases, the HLA heavy chain polypeptide is an HLA-A*0201 polypeptide with an A236C substitution. In some cases, the second polypeptide comprises, in order from N-terminus to C-terminus: i) a second MHC polypeptide; ii) an Ig Fc polypeptide; and iii) two immunomodulatory polypeptides, where the two immunomodulatory polypeptides have the same amino acid sequence. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, a peptide linker is between one or more of: i) the second MHC polypeptide and the Ig Fc polypeptide; ii) the epitope and the first MHC polypeptide; iii) the Ig Fc polypeptide and the immunomodulatory polypeptide; and (where the TMMP comprises two immunomodulatory polypeptides on the second polypeptide chain) iv) between the two immunomodulatory polypeptides. In some cases, the peptide linker comprises the amino acid sequence AAAGG (SEQ ID NO: 387). In some cases, the peptide linker comprises the amino acid sequence (GGGGS)n (SEQ ID NO:254), where n is an integer from 1 to 10 (e.g., where n is 2, 3, or 4). In some cases, the MUC1 peptide has the amino acid sequence STAPPVHNV (SEQ ID NO:587) and has a length of 9 amino acids. In some cases, the MUC1 peptide has the amino acid sequence SLAPPVHNV (SEQ ID NO:588) and has a length of 9 amino acids. In some cases, the MUC1 peptide has the amino acid sequence STAPPAHGV (SEQ ID NO:586) and has a length of 9 amino acids. In some cases, the MUC1 peptide has the amino acid sequence SLAPPAHGV (SEQ ID NO:589) and has a length of 9 amino acids.

In some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) at least one immunomodulatory polypeptide; ii) a second MHC polypeptide; and iii) an Ig Fc polypeptide. In some cases, the first MHC polypeptide is a β2M polypeptide; and the second MHC polypeptide is an HLA heavy chain polypeptide. In some cases, the HLA heavy chain polypeptide is an HLA-A*0201 polypeptide. In some cases, the HLA heavy chain polypeptide is an HLA-A*0201 polypeptide with an A236C substitution. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, the MUC1 peptide has the amino acid sequence STAPPVHNV (SEQ ID NO:587) and has a length of 9 amino acids. In some cases, the MUC1 peptide has the amino acid sequence SLAPPVHNV (SEQ ID NO:588) and has a length of 9 amino acids. In some cases, the MUC1 peptide has the amino acid sequence STAPPAHGV (SEQ ID NO:586) and has a length of 9 amino acids. In some cases, the MUC1 peptide has the amino acid sequence SLAPPAHGV (SEQ ID NO:589) and has a length of 9 amino acids.

In some cases, a TMMP of the present disclosure comprises: a) a first polypeptide comprising, in order from N-terminus to C-terminus: i) at least one immunomodulatory polypeptide; ii) a MUC1 peptide; and iii) a first MHC polypeptide; and b) a second polypeptide comprising, in order from N-terminus to C-terminus: i) a second MHC polypeptide; and ii) an Ig Fc polypeptide. In some cases, the first MHC polypeptide is a β2M polypeptide; and the second MHC polypeptide is an HLA heavy chain polypeptide. In some cases, the HLA heavy chain polypeptide is an HLA-A*0201 polypeptide. In some cases, the HLA heavy chain polypeptide is an HLA-A*0201 polypeptide with an A236C substitution. In some cases, the first polypeptide comprises, in order from N-terminus to C-terminus: i) two immunomodulatory polypeptides, where the two immunomodulatory polypeptides have the same amino acid sequence; ii) a peptide epitope; and iii) a first MHC polypeptide. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, a peptide linker is between one or more of: i) the second MHC polypeptide and the Ig Fc polypeptide; ii) the MUC1 peptide and the first MHC polypeptide; iii) the immunomodulatory polypeptide and the MUC1 peptide; and (where the TMMP comprises two immunomodulatory polypeptides on the first polypeptide chain) iv) between the two immunomodulatory polypeptides. In some cases, the peptide linker comprises the amino acid sequence AAAGG (SEQ ID NO: 387). In some cases, the peptide linker comprises the amino acid sequence (GGGGS)n (SEQ ID NO:254), where n is an integer from 1 to 10 (e.g., where n is 2, 3, or 4). In some cases, the MUC1 peptide has the amino acid sequence STAPPVHNV (SEQ ID NO:587) and has a length of 9 amino acids. In some cases, the MUC1 peptide has the amino acid sequence SLAPPVHNV (SEQ ID NO:588) and has a length of 9 amino acids. In some cases, the MUC1 peptide has the amino acid sequence STAPPAHGV (SEQ ID NO:586) and has a length of 9 amino acids. In some cases, the MUC1 peptide has the amino acid sequence SLAPPAHGV (SEQ ID NO:589) and has a length of 9 amino acids.

In some cases, a TMMP of the present disclosure comprises: a) a first polypeptide comprising, in order from N-terminus to C-terminus: i) a MUC1 peptide; and ii) a first MHC polypeptide; and b) a second polypeptide comprising, in order from N-terminus to C-terminus: i) a second MHC polypeptide; ii) at least one immunomodulatory polypeptide; and iii) an Ig Fc polypeptide. In some cases, the first MHC polypeptide is a β2M polypeptide; and the second MHC polypeptide is an HLA heavy chain polypeptide. In some cases, the HLA heavy chain polypeptide is an HLA-A*0201 polypeptide. In some cases, the HLA heavy chain polypeptide is an HLA-A*0201 polypeptide with an A236C substitution. In some cases, the second polypeptide comprises, in order from N-terminus to C-terminus: i) a second MHC polypeptide; ii) two immunomodulatory polypeptides, where the two immunomodulatory polypeptides have the same amino acid sequence; and iii) an Ig Fc polypeptide. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, a peptide linker is between one or more of: i) the second MHC polypeptide and the immunomodulatory polypeptide; ii) the immunomodulatory polypeptide and the Ig Fc polypeptide; iii) the MUC1 peptide and the first MHC polypeptide; iii) the first MHC polypeptide and the immunomodulatory polypeptide; and (where the TMMP comprises two immunomodulatory polypeptides on the second polypeptide chain) iv) between the two immunomodulatory polypeptides. In some cases, the peptide linker comprises the amino acid sequence AAAGG (SEQ ID NO: 387). In some cases, the peptide linker comprises the amino acid sequence (GGGGS)n (SEQ ID NO:254), where n is an integer from 1 to 10 (e.g., where n is 2, 3, or 4). In some cases, the MUC1 peptide has the amino acid sequence STAPPVHNV (SEQ ID NO:587) and has a length of 9 amino acids. In some cases, the MUC1 peptide has the amino acid sequence g147SLAPPVHNV (SEQ ID NO:588) and has a length of 9 amino acids. In some cases, the MUC1 peptide has the amino acid sequence STAPPAHGV (SEQ ID NO:586) and has a length of 9 amino acids. In some cases, the MUC1 peptide has the amino acid sequence SLAPPAHGV (SEQ ID NO:589) and has a length of 9 amino acids.

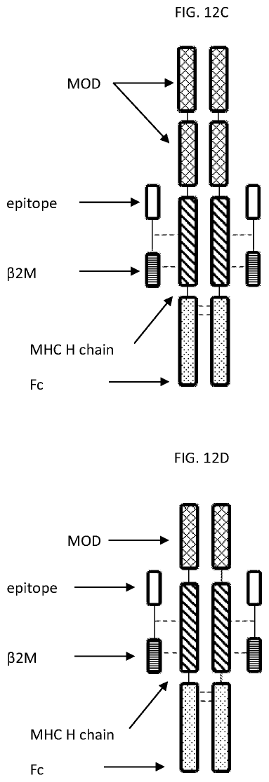

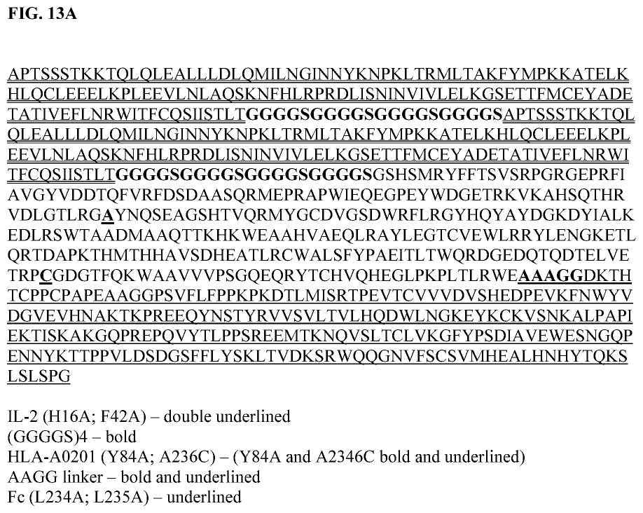

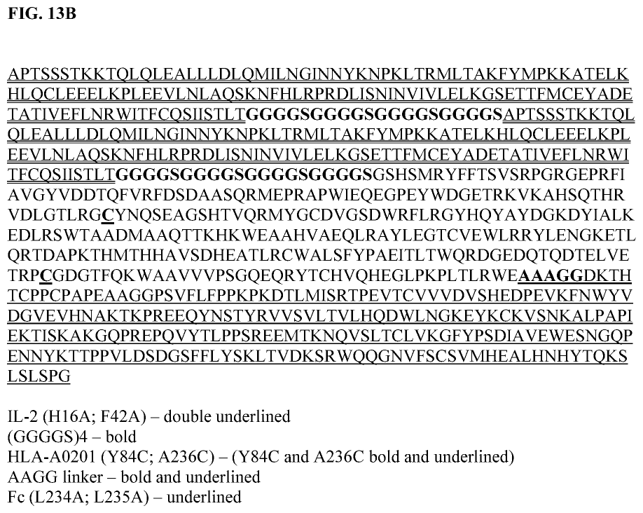

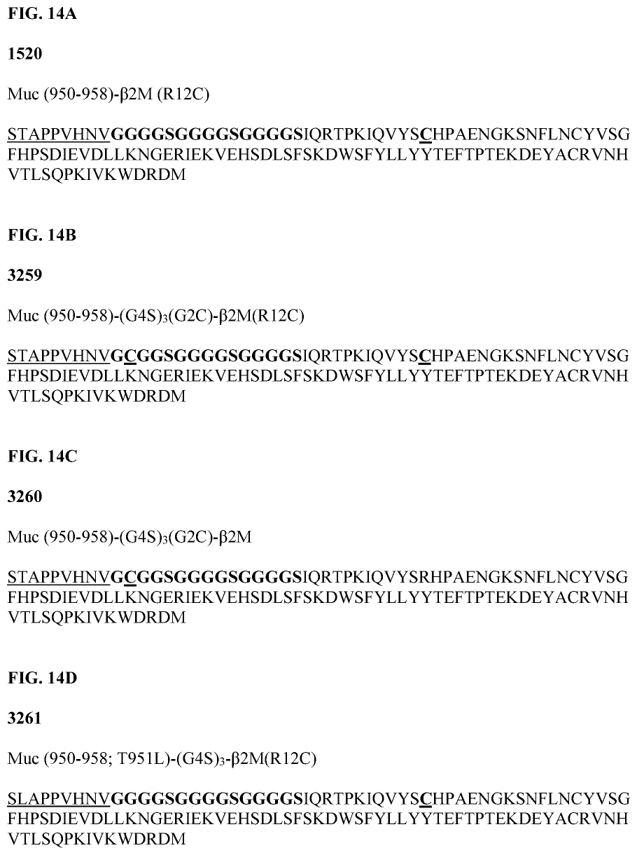

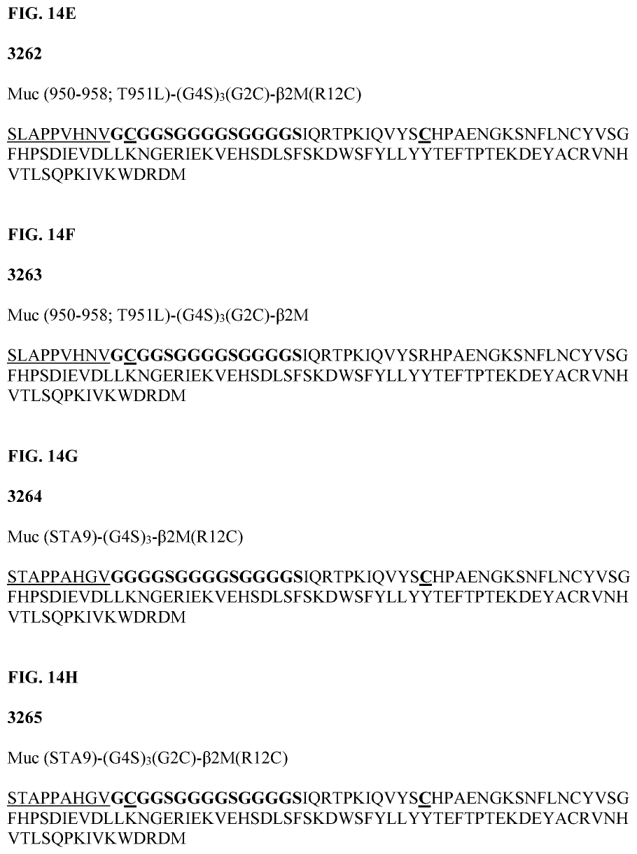

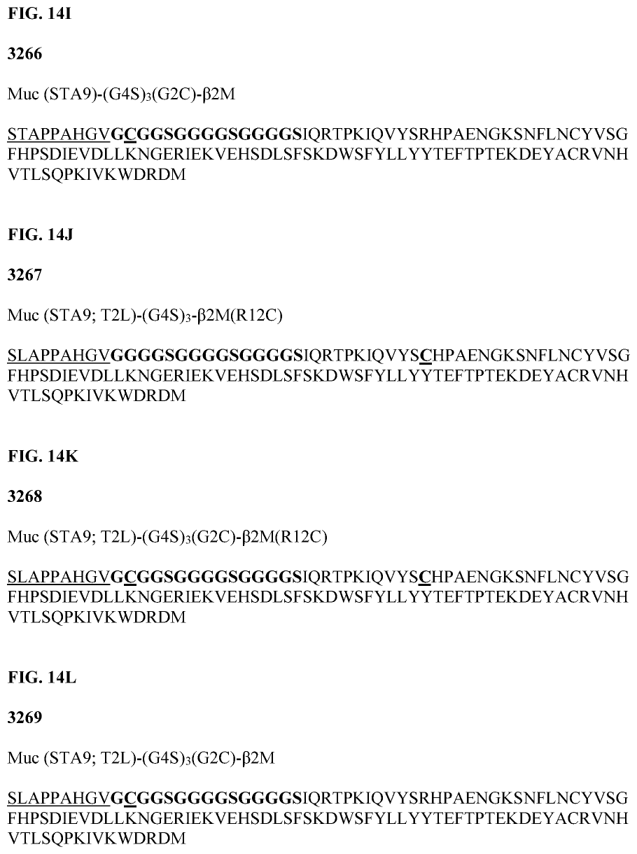

As one non-limiting example, a TMMP of the present disclosure can comprise: a) a first polypeptide comprising the amino acid sequence depicted in FIG. 13A; and b) a second polypeptide comprising the amino acid sequence depicted in FIG. 14A. As a further example, a TMMP of the present disclosure can comprise: a) a first polypeptide comprising the amino acid sequence depicted in FIG. 13A; and b) a second polypeptide comprising the amino acid sequence depicted in FIG. 14B. As a further example, a TMMP of the present disclosure can comprise: a) a first polypeptide comprising the amino acid sequence depicted in FIG. 13A; and b) a second polypeptide comprising the amino acid sequence depicted in FIG. 14C. As a further example, a TMMP of the present disclosure can comprise: a) a first polypeptide comprising the amino acid sequence depicted in FIG. 13A; and b) a second polypeptide comprising the amino acid sequence depicted in FIG. 14D. As a further example, a TMMP of the present disclosure can comprise: a) a first polypeptide comprising the amino acid sequence depicted in FIG. 13A; and b) a second polypeptide comprising the amino acid sequence depicted in FIG. 14E. As a further example, a TMMP of the present disclosure can comprise: a) a first polypeptide comprising the amino acid sequence depicted in FIG. 13A; and b) a second polypeptide comprising the amino acid sequence depicted in FIG. 14F. As a further example, a TMMP of the present disclosure can comprise: a) a first polypeptide comprising the amino acid sequence depicted in FIG. 13A; and b) a second polypeptide comprising the amino acid sequence depicted in FIG. 14G. As a further example, a TMMP of the present disclosure can comprise: a) a first polypeptide comprising the amino acid sequence depicted in FIG. 13A; and b) a second polypeptide comprising the amino acid sequence depicted in FIG. 14H. As a further example, a TMMP of the present disclosure can comprise: a) a first polypeptide comprising the amino acid sequence depicted in FIG. 13A; and b) a second polypeptide comprising the amino acid sequence depicted in FIG. 14I. As a further example, a TMMP of the present disclosure can comprise: a) a first polypeptide comprising the amino acid sequence depicted in FIG. 13A; and b) a second polypeptide comprising the amino acid sequence depicted in FIG. 14J. As a further example, a TMMP of the present disclosure can comprise: a) a first polypeptide comprising the amino acid sequence depicted in FIG. 13A; and b) a second polypeptide comprising the amino acid sequence depicted in FIG. 14K. As a further example, a TMMP of the present disclosure can comprise: a) a first polypeptide comprising the amino acid sequence depicted in FIG. 13A; and b) a second polypeptide comprising the amino acid sequence depicted in FIG. 14L.

As one non-limiting example, a TMMP of the present disclosure can comprise: a) a first polypeptide comprising the amino acid sequence depicted in FIG. 13B; and b) a second polypeptide comprising the amino acid sequence depicted in FIG. 14A. As a further example, a TMMP of the present disclosure can comprise: a) a first polypeptide comprising the amino acid sequence depicted in FIG. 13B; and b) a second polypeptide comprising the amino acid sequence depicted in FIG. 14B. As a further example, a TMMP of the present disclosure can comprise: a) a first polypeptide comprising the amino acid sequence depicted in FIG. 13B; and b) a second polypeptide comprising the amino acid sequence depicted in FIG. 14C. As a further example, a TMMP of the present disclosure can comprise: a) a first polypeptide comprising the amino acid sequence depicted in FIG. 13B; and b) a second polypeptide comprising the amino acid sequence depicted in FIG. 14D. As a further example, a TMMP of the present disclosure can comprise: a) a first polypeptide comprising the amino acid sequence depicted in FIG. 13B; and b) a second polypeptide comprising the amino acid sequence depicted in FIG. 14E. As a further example, a TMMP of the present disclosure can comprise: a) a first polypeptide comprising the amino acid sequence depicted in FIG. 13B; and b) a second polypeptide comprising the amino acid sequence depicted in FIG. 14F. As a further example, a TMMP of the present disclosure can comprise: a) a first polypeptide comprising the amino acid sequence depicted in FIG. 13B; and b) a second polypeptide comprising the amino acid sequence depicted in FIG. 14G. As a further example, a TMMP of the present disclosure can comprise: a) a first polypeptide comprising the amino acid sequence depicted in FIG. 13B; and b) a second polypeptide comprising the amino acid sequence depicted in FIG. 14H. As a further example, a TMMP of the present disclosure can comprise: a) a first polypeptide comprising the amino acid sequence depicted in FIG. 13B; and b) a second polypeptide comprising the amino acid sequence depicted in FIG. 14I. As a further example, a TMMP of the present disclosure can comprise: a) a first polypeptide comprising the amino acid sequence depicted in FIG. 13B; and b) a second polypeptide comprising the amino acid sequence depicted in FIG. 14J. As a further example, a TMMP of the present disclosure can comprise: a) a first polypeptide comprising the amino acid sequence depicted in FIG. 13B; and b) a second polypeptide comprising the amino acid sequence depicted in FIG. 14K. As a further example, a TMMP of the present disclosure can comprise: a) a first polypeptide comprising the amino acid sequence depicted in FIG. 13B; and b) a second polypeptide comprising the amino acid sequence depicted in FIG. 14L.

### Methods of Generating a Multimeric T-Cell Modulatory Polypeptide

The present disclosure provides a method of obtaining a TMMP comprising one or more variant immunomodulatory polypeptides that exhibit lower affinity for a cognate co-immunomodulatory polypeptide compared to the affinity of the corresponding parental wild-type immunomodulatory polypeptide for the co-immunomodulatory polypeptide, the method comprising: A) generating a library of TMMPs comprising a plurality of members, wherein each member comprises: a) a first polypeptide comprising: i) an epitope; and ii) a first major MHC polypeptide; and b) a second polypeptide comprising: i) a second MHC polypeptide; and ii) optionally an Ig Fc polypeptide or a non-Ig scaffold, wherein each member comprises a different variant immunomodulatory polypeptide on the first polypeptide, the second polypeptide, or both the first and the second polypeptide; B) determining the affinity of each member of the library for a cognate co-immunomodulatory polypeptide; and C) selecting a member that exhibits reduced affinity for the cognate co-immunomodulatory polypeptide. In some cases, the affinity is determined by bio-layer interferometry (BLI) using purified TMMP library members and the cognate co-immunomodulatory polypeptide. BLI methods are well known to those skilled in the art. A BLI assay is described above. See, e.g., Lad et al. (2015) J. Biomol. Screen. 20(4): 498-507; and Shah and Duncan (2014) J. Vis. Exp. 18:e51383.

The present disclosure provides a method of obtaining a TMMP that exhibits selective binding to a T-cell, the method comprising: A) generating a library of TMMPs comprising a plurality of members, wherein each member comprises: a) a first polypeptide comprising: i) an epitope; and ii) a first MHC polypeptide; and b) a second polypeptide comprising: i) a second MHC polypeptide; and ii) optionally an immunoglobulin (Ig) Fc polypeptide or a non-Ig scaffold, wherein each member comprises a different variant immunomodulatory polypeptide on the first polypeptide, the second polypeptide, or both the first and the second polypeptide, wherein the variant immunomodulatory polypeptide differs in amino acid sequence by from 1 amino acid to 10 amino acids from a parental wild-type immunomodulatory polypeptide; B) contacting a TMMP library member with a target T-cell expressing on its surface: i) a cognate co-immunomodulatory polypeptide that binds the parental wild-type immunomodulatory polypeptide; and ii) a T-cell receptor that binds to the epitope, wherein the TMMP library member comprises an epitope tag, such that the TMMP library member binds to the target T-cell; C) contacting the TMMP library member bound to the target T-cell with a fluorescently labeled binding agent that binds to the epitope tag, generating a TMMP library member/target T-cell/binding agent complex; D) measuring the mean fluorescence intensity (MFI) of the TMMP library member/target T-cell/binding agent complex using flow cytometry, wherein the MFI measured over a range of concentrations of the TMMP library member provides a measure of the affinity and apparent avidity; and E) selecting a TMMP library member that selectively binds the target T cell, compared to binding of the TMMP library member to a control T cell that comprises: i) the cognate co-immunomodulatory polypeptide that binds the parental wild-type immunomodulatory polypeptide; and ii) a T-cell receptor that binds to an epitope other than the epitope present in the TMMP library member. In some cases, a TMMP library member that is identified as selectively binds to a target T cell is isolated from the library.

In some cases, a parental wild-type immunomodulatory polypeptide and cognate immunomodulatory polypeptide pairs are selected from:

IL-2 and IL-2 receptor;

4-1BBL and 4-1BB;

PD-L1 and PD-1;

CD70 and CD27;

TGFβ and TGFβ receptor;

CD80 and CD28;

CD86 and CD28;

OX40L and OX40;

FasL and Fas;

ICOS-L and ICOS;

ICAM and LFA-1;

JAG1 and Notch;

JAG1 and CD46;

CD80 and CTLA4; and

CD86 and CTLA4.

The present disclosure provides a method of obtaining a TMMP comprising one or more variant immunomodulatory polypeptides that exhibit reduced affinity for a cognate co-immunomodulatory polypeptide compared to the affinity of the corresponding parental wild-type immunomodulatory polypeptide for the co-immunomodulatory polypeptide, the method comprising selecting, from a library of TMMPs comprising a plurality of members, a member that exhibits reduced affinity for the cognate co-immunomodulatory polypeptide, wherein the plurality of member comprises: a) a first polypeptide comprising: i) an epitope; and ii) a first MHC polypeptide; and b) a second polypeptide comprising: i) a second MHC polypeptide; and ii) optionally an Ig Fc polypeptide or a non-Ig scaffold, wherein the members of the library comprise a plurality of variant immunomodulatory polypeptide present in the first polypeptide, the second polypeptide, or both the first and the second polypeptide. In some cases, the selecting step comprises determining the affinity, using bio-layer interferometry, of binding between TMMP library members and the cognate co-immunomodulatory polypeptide. In some cases, the TMMP is as described above.

In some cases, the method further comprises: a) contacting the selected TMMP library member with a target T-cell expressing on its surface: i) a cognate co-immunomodulatory polypeptide that binds the parental wild-type immunomodulatory polypeptide; and ii) a T-cell receptor that binds to the epitope, wherein the TMMP library member comprises an epitope tag, such that the TMMP library member binds to the target T-cell; b) contacting the selected TMMP library member bound to the target T-cell with a fluorescently labeled binding agent that binds to the epitope tag, generating a selected TMMP library member/target T-cell/binding agent complex; and c) measuring the mean fluorescence intensity (MFI) of the selected TMMP library member/target T-cell/binding agent complex using flow cytometry, wherein the MFI measured over a range of concentrations of the selected TMMP library member provides a measure of the affinity and apparent avidity. A selected TMMP library member that selectively binds the target T cell, compared to binding of the TMMP library member to a control T cell that comprises: i) the cognate co-immunomodulatory polypeptide that binds the parental wild-type immunomodulatory polypeptide; and ii) a T-cell receptor that binds to an epitope other than the epitope present in the TMMP library member, is identified as selectively binding to the target T cell. In some cases, the binding agent is an antibody specific for the epitope tag. In some cases, the variant immunomodulatory polypeptide comprises from 1 to 20 amino acid substitutions (e.g., 1, 2, 3, 4, 5, 6, 7, 8, 9, 10, 11, 12, 13, 14, 15, 16, 17, 18, 19, or 20 amino acid substitutions) compared to the corresponding parental wild-type immunomodulatory polypeptide. In some cases, the TMMP comprises two variant immunomodulatory polypeptides. In some cases, the two variant immunomodulatory polypeptides comprise the same amino acid sequence. In some cases, the first polypeptide comprises one of the two variant immunomodulatory polypeptides and wherein the second polypeptide comprises the second of the two variant immunomodulatory polypeptides. In some cases, the two variant immunomodulatory polypeptides are on the same polypeptide chain of the TMMP. In some cases, the two variant immunomodulatory polypeptides are on the first polypeptide of the TMMP. In some cases, the two variant immunomodulatory polypeptides are on the second polypeptide of the TMMP.

In some cases, the method further comprises isolating the selected TMMP library member from the library. In some cases, the method further comprises providing a nucleic acid comprising a nucleotide sequence encoding the selected TMMP library member. In some cases, the nucleic acid is present in a recombinant expression vector. In some cases, the nucleotide sequence is operably linked to a transcriptional control element that is functional in a eukaryotic cell. In some cases, the method further comprises introducing the nucleic acid into a eukaryotic host cell, and culturing the cell in a liquid medium to synthesize the encoded selected TMMP library member in the cell. In some cases, the method further comprises isolating the synthesized selected TMMP library member from the cell or from liquid culture medium comprising the cell. In some cases, the selected TMMP library member comprises an Ig Fc polypeptide. In some cases, the method further comprises conjugating a drug to the Ig Fc polypeptide. In some cases, the drug is a cytotoxic agent is selected from maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. In some cases, the drug is a retinoid. In some cases, the parental wild-type immunomodulatory polypeptide and the cognate immunomodulatory polypeptides are selected from: IL-2 and IL-2 receptor; 4-1BBL and 4-1BB; PD-L1 and PD-1; CD70 and CD27; TGFβ and TGFβ receptor; CD80 and CD28; CD86 and CD28; OX40L and OX40; FasL and Fas; ICOS-L and ICOS; ICAM and LFA-1; JAG1 and Notch; JAG1 and CD46; CD80 and CTLA4; and CD86 and CTLA4.

The present disclosure provides a method of obtaining a TMMP comprising one or more variant immunomodulatory polypeptides that exhibit reduced affinity for a cognate co-immunomodulatory polypeptide compared to the affinity of the corresponding parental wild-type immunomodulatory polypeptide for the co-immunomodulatory polypeptide, the method comprising: A) providing a library of TMMPs comprising a plurality of members, wherein the plurality of member comprises: a) a first polypeptide comprising: i) an epitope; and ii) a first MHC polypeptide; and b) a second polypeptide comprising: i) a second MHC polypeptide; and ii) optionally an Ig Fc polypeptide or a non-Ig scaffold, wherein the members of the library comprise a plurality of variant immunomodulatory polypeptide present in the first polypeptide, the second polypeptide, or both the first and the second polypeptide; and B) selecting from the library a member that exhibits reduced affinity for the cognate co-immunomodulatory polypeptide. In some cases, the selecting step comprises determining the affinity, using bio-layer interferometry, of binding between TMMP library members and the cognate co-immunomodulatory polypeptide. In some cases, the TMMP is as described above.

In some cases, the method further comprises: a) contacting the selected TMMP library member with a target T-cell expressing on its surface: i) a cognate co-immunomodulatory polypeptide that binds the parental wild-type immunomodulatory polypeptide; and ii) a T-cell receptor that binds to the epitope, wherein the TMMP library member comprises an epitope tag, such that the TMMP library member binds to the target T-cell; b) contacting the selected TMMP library member bound to the target T-cell with a fluorescently labeled binding agent that binds to the epitope tag, generating a selected TMMP library member/target T-cell/binding agent complex; and c) measuring the mean fluorescence intensity (MFI) of the selected TMMP library member/target T-cell/binding agent complex using flow cytometry, wherein the MFI measured over a range of concentrations of the selected TMMP library member provides a measure of the affinity and apparent avidity. A selected TMMP library member that selectively binds the target T cell, compared to binding of the TMMP library member to a control T cell that comprises: i) the cognate co-immunomodulatory polypeptide that binds the parental wild-type immunomodulatory polypeptide; and ii) a T-cell receptor that binds to an epitope other than the epitope present in the TMMP library member, is identified as selectively binding to the target T cell. In some cases, the binding agent is an antibody specific for the epitope tag. In some cases, the variant immunomodulatory polypeptide comprises from 1 to 20 amino acid substitutions (e.g., 1, 2, 3, 4, 5, 6, 7, 8, 9, 10, 11, 12, 13, 14, 15, 16, 17, 18, 19, or 20 amino acid substitutions) compared to the corresponding parental wild-type immunomodulatory polypeptide. In some cases, the TMMP comprises two variant immunomodulatory polypeptides. In some cases, the two variant immunomodulatory polypeptides comprise the same amino acid sequence. In some cases, the first polypeptide comprises one of the two variant immunomodulatory polypeptides and wherein the second polypeptide comprises the second of the two variant immunomodulatory polypeptides. In some cases, the two variant immunomodulatory polypeptides are on the same polypeptide chain of the TMMP. In some cases, the two variant immunomodulatory polypeptides are on the first polypeptide of the TMMP. In some cases, the two variant immunomodulatory polypeptides are on the second polypeptide of the TMMP.

In some cases, the method further comprises isolating the selected TMMP library member from the library. In some cases, the method further comprises providing a nucleic acid comprising a nucleotide sequence encoding the selected TMMP library member. In some cases, the nucleic acid is present in a recombinant expression vector. In some cases, the nucleotide sequence is operably linked to a transcriptional control element that is functional in a eukaryotic cell. In some cases, the method further comprises introducing the nucleic acid into a eukaryotic host cell, and culturing the cell in a liquid medium to synthesize the encoded selected TMMP library member in the cell. In some cases, the method further comprises isolating the synthesized selected TMMP library member from the cell or from liquid culture medium comprising the cell. In some cases, the selected TMMP library member comprises an Ig Fc polypeptide. In some cases, the method further comprises conjugating a drug to the Ig Fc polypeptide. In some cases, the drug is a cytotoxic agent is selected from maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. In some cases, the drug is a retinoid. In some cases, the parental wild-type immunomodulatory polypeptide and the cognate immunomodulatory polypeptides are selected from IL-2 and IL-2 receptor; 4-1BBL and 4-1BB; PD-L1 and PD-1; TGFβ and TGFβ receptor; CD80 and CD28; CD86 and CD28; OX40L and OX40; FasL and Fas; ICOS-L and ICOS; CD70 and CD27; ICAM and LFA-1; JAG1 and Notch; JAG1 and CD46; CD80 and CTLA4; and CD86 and CTLA4.

### Nucleic Acids

The present disclosure provides a nucleic acid comprising a nucleotide sequence encoding a TMMP of the present disclosure. The present disclosure provides a nucleic acid comprising a nucleotide sequence encoding a TMMP of the present disclosure.

The present disclosure provides nucleic acids comprising nucleotide sequences encoding a TMMP of the present disclosure. In some cases, the individual polypeptide chains of a TMMP of the present disclosure are encoded in separate nucleic acids. In some cases, all polypeptide chains of a TMMP of the present disclosure are encoded in a single nucleic acid. In some cases, a first nucleic acid comprises a nucleotide sequence encoding a first polypeptide of a TMMP of the present disclosure; and a second nucleic acid comprises a nucleotide sequence encoding a second polypeptide of a TMMP of the present disclosure. In some cases, single nucleic acid comprises a nucleotide sequence encoding a first polypeptide of a TMMP of the present disclosure and a second polypeptide of a TMMP of the present disclosure.

### Separate Nucleic Acids Encoding Individual Polypeptide Chains of a Multimeric Polypeptide

The present disclosure provides nucleic acids comprising nucleotide sequences encoding a TMMP of the present disclosure. As noted above, in some cases, the individual polypeptide chains of a TMMP of the present disclosure are encoded in separate nucleic acids. In some cases, nucleotide sequences encoding the separate polypeptide chains of a TMMP of the present disclosure are operably linked to transcriptional control elements, e.g., promoters, such as promoters that are functional in a eukaryotic cell, where the promoter can be a constitutive promoter or an inducible promoter.

The present disclosure provides a first nucleic acid and a second nucleic acid, where the first nucleic acid comprises a nucleotide sequence encoding a first polypeptide of a TMMP of the present disclosure, where the first polypeptide comprises, in order from N-terminus to C-terminus: a) an epitope (e.g., a T-cell epitope); b) a first MHC polypeptide; and c) an immunomodulatory polypeptide (e.g., a reduced-affinity variant, as described above); and where the second nucleic acid comprises a nucleotide sequence encoding a second polypeptide of a TMMP of the present disclosure, where the second polypeptide comprises, in order from N-terminus to C-terminus: a) a second MHC polypeptide; and b) an Ig Fc polypeptide. Suitable T-cell epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

The present disclosure provides a first nucleic acid and a second nucleic acid, where the first nucleic acid comprises a nucleotide sequence encoding a first polypeptide of a TMMP of the present disclosure, where the first polypeptide comprises, in order from N-terminus to C-terminus: a) an epitope (e.g., a T-cell epitope); and b) a first MHC polypeptide; and where the second nucleic acid comprises a nucleotide sequence encoding a second polypeptide of a TMMP of the present disclosure, where the second polypeptide comprises, in order from N-terminus to C-terminus: a) an immunomodulatory polypeptide (e.g., a reduced-affinity variant as described above); b) a second MHC polypeptide; and c) an Ig Fc polypeptide. Suitable T-cell epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a Multimeric Polypeptide

The present disclosure provides a nucleic acid comprising nucleotide sequences encoding at least the first polypeptide and the second polypeptide of a TMMP of the present disclosure. In some cases, where a TMMP of the present disclosure includes a first, second, and third polypeptide, the nucleic acid includes a nucleotide sequence encoding the first, second, and third polypeptides. In some cases, the nucleotide sequences encoding the first polypeptide and the second polypeptide of a TMMP of the present disclosure includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMMP of the present disclosure includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMMP of the present disclosure includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a TMMP of the present disclosure; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a TMMP of the present disclosure; and a second nucleic acid (e.g., a recombinant expression vector, an mRNA, a viral RNA, etc.) comprises a nucleotide sequence encoding a second polypeptide chain of a TMMP of the present disclosure. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

The present disclosure provides a nucleic acid comprising a nucleotide sequence encoding a recombinant polypeptide, where the recombinant polypeptide comprises, in order from N-terminus to C-terminus: a) an epitope (e.g., a T-cell epitope); b) a first MHC polypeptide; c) an immunomodulatory polypeptide (e.g., a reduced-affinity variant as described above); d) a proteolytically cleavable linker; e) a second MHC polypeptide; and f) an immunoglobulin (Ig) Fc polypeptide. The present disclosure provides a nucleic acid comprising a nucleotide sequence encoding a recombinant polypeptide, where the recombinant polypeptide comprises, in order from N-terminus to C-terminus: a) a first leader peptide; b) the epitope; c) the first MHC polypeptide; d) the immunomodulatory polypeptide (e.g., a reduced-affinity variant as described above); e) the proteolytically cleavable linker; f) a second leader peptide; g) the second MHC polypeptide; and h) the Ig Fc polypeptide. The present disclosure provides a nucleic acid comprising a nucleotide sequence encoding a recombinant polypeptide, where the recombinant polypeptide comprises, in order from N-terminus to C-terminus: a) an epitope; b) a first MHC polypeptide; c) a proteolytically cleavable linker; d) an immunomodulatory polypeptide (e.g., a reduced-affinity variant as described above); e) a second MHC polypeptide; and f) an Ig Fc polypeptide. In some cases, the first leader peptide and the second leader peptide are a β2-M leader peptide. In some cases, the nucleotide sequence is operably linked to a transcriptional control element. In some cases, the transcriptional control element is a promoter that is functional in a eukaryotic cell.

Suitable MHC polypeptides are described above. In some cases, the first MHC polypeptide is a β2-microglobulin polypeptide; and wherein the second MHC polypeptide is an MHC class I heavy chain polypeptide. In some cases, the β2-microglobulin polypeptide comprises an amino acid sequence having at least 85% amino acid sequence identity to a β2M amino acid sequence depicted in FIG. 4. In some cases, the MHC class I heavy chain polypeptide is an HLA-A, HLA-B, HLA-C, HLA-E, HLA-F, HLA-G, HLA-K, or HLA-L heavy chain.

Suitable Fc polypeptides are described above. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% amino acid sequence identity to an amino acid sequence depicted in FIGS. 3A-3G.

Suitable immunomodulatory polypeptides are described above.

Suitable proteolytically cleavable linkers are described above. In some cases, the proteolytically cleavable linker comprises an amino acid sequence selected from: a) LEVLFQGP (SEQ ID NO:556); b) ENLYTQS (SEQ ID NO:557); c) DDDDK (SEQ ID NO:558); d) LVPR (SEQ ID NO:559); and e) GSGATNFSLLKQAGDVEENPGP (SEQ ID NO:560).

In some cases, a linker between the epitope and the first MHC polypeptide comprises a first Cys residue, and the second MHC polypeptide comprises an amino acid substitution to provide a second Cys residue, such that the first and the second Cys residues provide for a disulfide linkage between the linker and the second MHC polypeptide. In some cases, first MHC polypeptide comprises an amino acid substitution to provide a first Cys residue, and the second MHC polypeptide comprises an amino acid substitution to provide a second Cys residue, such that the first Cys residue and the second Cys residue provide for a disulfide linkage between the first MHC polypeptide and the second MHC polypeptide.

### Recombinant Expression Vectors

The present disclosure provides recombinant expression vectors comprising nucleic acids of the present disclosure. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some cases, a nucleotide sequence encoding a DNA-targeting RNA and/or a site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid ossf the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2-M.

In some cases, the host cell is a mammalian cell that has been genetically modified such that it does not synthesize endogenous MHC Class I heavy chain. In some cases, the host cell is a mammalian cell that has been genetically modified such that it does not synthesize endogenous MHC β2-M and such that it does not synthesize endogenous MHC Class I heavy chain.

### Compositions

The present disclosure provides compositions, including pharmaceutical compositions, comprising a TMMP (synTac) of the present disclosure. The present disclosure provides compositions, including pharmaceutical compositions, comprising a TMMP of the present disclosure. The present disclosure provides compositions, including pharmaceutical compositions, comprising a nucleic acid or a recombinant expression vector of the present disclosure.

### Compositions Comprising a Multimeric Polypeptide

A composition of the present disclosure can comprise, in addition to a TMMP of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise a TMMP of the present disclosure, and a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, and the like. The composition may be formulated according to the various routes of administration described below.

Where a TMMP of the present disclosure is administered as an injectable (e.g. subcutaneously, intraperitoneally, intramuscularly, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the TMMP following administration. For example, the TMMP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a TMMP of the present disclosure in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

The present disclosure provides compositions, including pharmaceutical compositions, comprising a TMMP of the present disclosure. A composition can comprise: a) a TMMP of the present disclosure; and b) an excipient, as described above. In some cases, the excipient is a pharmaceutically acceptable excipient.

In some cases, a TMMP of the present disclosure is present in a liquid composition. Thus, the present disclosure provides compositions (e.g., liquid compositions, including pharmaceutical compositions) comprising a TMMP of the present disclosure. In some cases, a composition of the present disclosure comprises: a) a TMMP of the present disclosure; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a TMMP of the present disclosure; and b) saline (e.g., 0.9% NaCl), where the composition is sterile and is free of detectable pyrogens and/or other toxins.

### Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding a TMMP; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some cases, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

### Methods of Modulating T Cell Activity

The present disclosure provides a method of selectively modulating the activity of an epitope-specific T cell (e.g., a T cell specific for a MUC1 epitope), the method comprising contacting the T cell with a TMMP of the present disclosure, where contacting the T cell with a TMMP of the present disclosure selectively modulates the activity of the epitope-specific T cell. In some cases, the contacting occurs in vitro. In some cases, the contacting occurs in vivo. In some cases, the contacting occurs ex vivo.

In some cases, e.g., where the target T cell is a CD8⁺ T cell, the TMMP comprises Class I MHC polypeptides (e.g., β2-microglobulin and Class I MHC heavy chain).

Where a TMMP of the present disclosure includes an immunomodulatory polypeptide that is an activating polypeptide, contacting the T cell with the TMMP activates the epitope-specific T cell. In some instances, the epitope-specific T cell is a T cell that is specific for an epitope present on a cancer cell, and contacting the epitope-specific T cell with the TMMP increases cytotoxic activity of the T cell toward the cancer cell. In some instances, the epitope-specific T cell is a T cell that is specific for an epitope present on a cancer cell, and contacting the epitope-specific T cell with the TMMP increases the number of the epitope-specific T cells.

The present disclosure provides a method of modulating an immune response in an individual, the method comprising administering to the individual an effective amount of a TMMP of the present disclosure. Administering the TMMP induces an epitope-specific T cell response (e.g., cancer epitope-specific T-cell response; a virus epitope-specific) and an epitope-non-specific T cell response, where the ratio of the epitope-specific T cell response to the epitope-non-specific T cell response is at least 2:1. In some cases, the ratio of the epitope-specific T cell response to the epitope-non-specific T cell response is at least 5:1. In some cases, the ratio of the epitope-specific T cell response to the epitope-non-specific T cell response is at least 10:1. In some cases, the ratio of the epitope-specific T cell response to the epitope-non-specific T cell response is at least 25:1. In some cases, the ratio of the epitope-specific T cell response to the epitope-non-specific T cell response is at least 50:1. In some cases, the ratio of the epitope-specific T cell response to the epitope-non-specific T cell response is at least 100:1. In some cases, the individual is a human. In some cases, the modulating increases a cytotoxic T-cell response to a cancer cell, e.g., a cancer cell expressing an antigen that displays the same epitope displayed by the peptide epitope present in the TMMP. In some cases, the administering is intravenous, subcutaneous, intramuscular, systemic, intralymphatic, distal to a treatment site, local, or at or near a treatment site.

The present disclosure provides a method of delivering a costimulatory (i.e., immunomodulatory) polypeptide selectively to target T cell, the method comprising contacting a mixed population of T cells with a TMMP of the present disclosure, where the mixed population of T cells comprises the target T cell and non-target T cells, where the target T cell is specific for the epitope present within the TMMP (e.g., where the target T cell is specific for the epitope present within the TMMP), and where the contacting step delivers the one or more costimulatory polypeptides (immunomodulatory polypeptides) present within the TMMP to the target T cell. In some cases, the population of T cells is in vitro. In some cases, the population of T cells is in vivo in an individual. In some cases, the method comprises administering the TMMP to the individual. In some case, the T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure provides a method of detecting, in a mixed population of T cells obtained from an individual, the presence of a target T cell that binds an epitope of interest (e.g., a cancer epitope, a virus epitope), the method comprising: a) contacting in vitro the mixed population of T cells with a TMMP of the present disclosure, wherein the TMMP comprises the epitope of interest (e.g., the cancer epitope, the virus epitope); and b) detecting activation and/or proliferation of T cells in response to said contacting, wherein activated and/or proliferated T cells indicates the presence of the target T cell.

### Treatment Methods

The present disclosure provides a method of treatment of an individual, the method comprising administering to the individual an amount of a TMMP of the present disclosure, or one or more nucleic acids encoding the TMMP, effective to treat the individual. Also provided is a TMMP of the present disclosure for use in a method of treatment of the human or animal body. In some cases, a treatment method of the present disclosure comprises administering to an individual in need thereof one or more recombinant expression vectors comprising nucleotide sequences encoding a TMMP of the present disclosure. In some cases, a treatment method of the present disclosure comprises administering to an individual in need thereof one or more mRNA molecules comprising nucleotide sequences encoding a TMMP of the present disclosure. In some cases, a treatment method of the present disclosure comprises administering to an individual in need thereof a TMMP of the present disclosure. Conditions that can be treated include, e.g., cancer, such as a cancer that overexpresses MUC1.

In some cases, a TMMP of the present disclosure, when administered to an individual in need thereof, induces both an epitope-specific T cell response and an epitope non-specific T cell response. In other words, in some cases, a TMMP of the present disclosure, when administered to an individual in need thereof, induces an epitope-specific T cell response by modulating the activity of a first T cell that displays both: i) a TCR specific for the epitope present in the TMMP; ii) a co-immunomodulatory polypeptide that binds to the immunomodulatory polypeptide present in the TMMP; and induces an epitope non-specific T cell response by modulating the activity of a second T cell that displays: i) a TCR specific for an epitope other than the epitope present in the TMMP; and ii) a co-immunomodulatory polypeptide that binds to the immunomodulatory polypeptide present in the TMMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; iv) inhibiting activity of an autoreactive T cell; and the like.

The combination of the reduced affinity of the immunomodulatory polypeptide for its cognate co-immunomodulatory polypeptide, and the affinity of the epitope for a TCR, provides for enhanced selectivity of a TMMP of the present disclosure. Thus, for example, a TMMP of the present disclosure binds with higher avidity to a first T cell that displays both: i) a TCR specific for the epitope present in the TMMP; and ii) a co-immunomodulatory polypeptide that binds to the immunomodulatory polypeptide present in the TMMP, compared to the avidity to which it binds to a second T cell that displays: i) a TCR specific for an epitope other than the epitope present in the TMMP; and ii) a co-immunomodulatory polypeptide that binds to the immunomodulatory polypeptide present in the TMMP.

The present disclosure provides a method of selectively modulating the activity of an epitope-specific T cell in an individual, the method comprising administering to the individual an effective amount of a TMMP of the present disclosure, or one or more nucleic acids (e.g., expression vectors; mRNA; etc.) comprising nucleotide sequences encoding the TMMP, where the TMMP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMMP of the present disclosure.

In some cases, the immunomodulatory polypeptide (“MOD”) is an activating polypeptide, and the TMMP activates the epitope-specific T cell. In some cases, the TMMP increases the activity of a T cell specific for the MUC1 epitope. In some cases, the MOD is an activating polypeptide, and the TMMP activates an epitope-specific T-cell (e.g., a T-cell specific for a MUC1 epitope). In some cases, the T cells are T-helper cells (CD4⁺ cells), cytotoxic T-cells (CD8⁺ cells), or NK-T-cells. In some cases, the epitope is a cancer epitope, and the TMMP increases the activity of a T-cell specific for a cancer cell expressing the MUC1 cancer epitope (e.g., T-helper cells (CD4⁺ cells), cytotoxic T-cells (CD8⁺ cells), and/or NK-T-cells). Activation of CD4⁺ T cells can include increasing proliferation of CD4⁺ T cells and/or inducing or enhancing release cytokines by CD4⁺ T cells. Activation of NK-T-cells and/or CD8+ cells can include: increasing proliferation of NK-T-cells and/or CD8+ cells; and/or inducing release of cytokines such as interferon γ by NK-T-cells and/or CD8+ cells.

A TMMP of the present disclosure can be administered to an individual in need thereof to treat a cancer in the individual, where the cancer expresses the MUC1 peptide present in the TMMP. For example, the cancer can be one in which the cancer cells over-express MUC1. The present disclosure provides a method of treating cancer in an individual, the method comprising administering to the individual an effective amount of a TMMP of the present disclosure, or one or more nucleic acids (e.g., expression vectors; mRNA; etc.) comprising nucleotide sequences encoding the TMMP, where the TMMP comprises a T-cell epitope that is a MUC1 epitope, and where the TMMP comprises a stimulatory immunomodulatory polypeptide. In some cases, an “effective amount” of a TMMP of the present disclosure is an amount that, when administered in one or more doses to an individual in need thereof, reduces the number of cancer cells in the individual. For example, in some cases, an “effective amount” of a TMMP of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the TMMP, or in the absence of administration with the TMMP. In some cases, an “effective amount” of a TMMP of the present disclosure is an amount that, when administered in one or more doses to an individual in need thereof, reduces the number of cancer cells in the individual to undetectable levels.

In some cases, an “effective amount” of a TMMP of the present disclosure is an amount that, when administered in one or more doses to an individual in need thereof, reduces the tumor mass in the individual. For example, in some cases, an “effective amount” of a TMMP of the present disclosure is an amount that, when administered in one or more doses to an individual in need thereof (an individual having a tumor), reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the TMMP, or in the absence of administration with the TMMP. In some cases, an “effective amount” of a TMMP of the present disclosure is an amount that, when administered in one or more doses to an individual in need thereof (an individual having a tumor), reduces the tumor volume in the individual. For example, in some cases, an “effective amount” of a TMMP of the present disclosure is an amount that, when administered in one or more doses to an individual in need thereof (an individual having a tumor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the TMMP, or in the absence of administration with the TMMP. In some cases, an “effective amount” of a TMMP of the present disclosure is an amount that, when administered in one or more doses to an individual in need thereof, increases survival time of the individual. For example, in some cases, an “effective amount” of a TMMP of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the TMMP.

Cancers that can be treated with a method of the present disclosure include cancers in which the cancer cells overexpress MUC1, e.g., cancer cells that overexpress hypoglycosylated MUC1. Examples include adenocarcinomas and hematological malignancies. Examples of cancers that can be treated with a method of the present disclosure include multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach chancer; urothelial cancer; cervical cancer; and ovarian endometrial cancer.

As noted above, in some cases, in carrying out a subject treatment method, a TMMP of the present disclosure is administered to an individual in need thereof, as the TMMP per se. In other instances, in carrying out a subject treatment method, one or more nucleic acids comprising nucleotide sequences encoding a TMMP of the present disclosure is/are administering to an individual in need thereof. Thus, in other instances, one or more nucleic acids of the present disclosure, e.g., one or more recombinant expression vectors of the present disclosure, is/are administered to an individual in need thereof.

### Formulations

Suitable formulations are described above, where suitable formulations include a pharmaceutically acceptable excipient. In some cases, a suitable formulation comprises: a) a TMMP of the present disclosure; and b) a pharmaceutically acceptable excipient. In some cases, a suitable formulation comprises: a) a nucleic acid comprising a nucleotide sequence encoding a TMMP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a TMMP of the present disclosure; b) a second nucleic acid comprising a nucleotide sequence encoding the second polypeptide of a TMMP of the present disclosure; and c) a pharmaceutically acceptable excipient. In some cases, a suitable formulation comprises: a) a recombinant expression vector comprising a nucleotide sequence encoding a TMMP of the present disclosure; and b) a pharmaceutically acceptable excipient. In some cases, a suitable formulation comprises: a) a first recombinant expression vector comprising a nucleotide sequence encoding the first polypeptide of a TMMP of the present disclosure; b) a second recombinant expression vector comprising a nucleotide sequence encoding the second polypeptide of a TMMP of the present disclosure; and c) a pharmaceutically acceptable excipient.

Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMMP of the present disclosure may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A TMMP of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a TMMP of the present disclosure is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMMP of the present disclosure is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific TMMP, the severity of the symptoms and the susceptibility of the subject to side effects. Preferred dosages for a given compound are readily determinable by those of skill in the art by a variety of means.

In some cases, multiple doses of a TMMP of the present disclosure, a nucleic acid of the present disclosure, or a recombinant expression vector of the present disclosure are administered. The frequency of administration of a TMMP of the present disclosure, a nucleic acid of the present disclosure, or a recombinant expression vector of the present disclosure can vary depending on any of a variety of factors, e.g., severity of the symptoms, etc. For example, in some cases, a TMMP of the present disclosure, a nucleic acid of the present disclosure, or a recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a TMMP of the present disclosure, a nucleic acid of the present disclosure, or a recombinant expression vector of the present disclosure, e.g., the period of time over which a TMMP of the present disclosure, a nucleic acid of the present disclosure, or a recombinant expression vector of the present disclosure is administered, can vary, depending on any of a variety of factors, e.g., patient response, etc. For example, a TMMP of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An active agent (a TMMP of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intralymphatic, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the TMMP and/or the desired effect. A TMMP of the present disclosure, or a nucleic acid or recombinant expression vector of the present disclosure, can be administered in a single dose or in multiple doses.

In some cases, a TMMP of the present disclosure, a nucleic acid of the present disclosure, or a recombinant expression vector of the present disclosure is administered intravenously. In some cases, a TMMP of the present disclosure, a nucleic acid of the present disclosure, or a recombinant expression vector of the present disclosure is administered intramuscularly. In some cases, a TMMP of the present disclosure, a nucleic acid of the present disclosure, or a recombinant expression vector of the present disclosure is administered intralymphatically. In some cases, a TMMP of the present disclosure, a nucleic acid of the present disclosure, or a recombinant expression vector of the present disclosure is administered locally. In some cases, a TMMP of the present disclosure, a nucleic acid of the present disclosure, or a recombinant expression vector of the present disclosure is administered intratumorally. In some cases, a TMMP of the present disclosure, a nucleic acid of the present disclosure, or a recombinant expression vector of the present disclosure is administered peritumorally. In some cases, a TMMP of the present disclosure, a nucleic acid of the present disclosure, or a recombinant expression vector of the present disclosure is administered intracranially. In some cases, a TMMP of the present disclosure, a nucleic acid of the present disclosure, or a recombinant expression vector of the present disclosure is administered subcutaneously.

In some cases, a TMMP of the present disclosure is administered intravenously. In some cases, a TMMP of the present disclosure is administered intramuscularly. In some cases, a TMMP of the present disclosure is administered locally. In some cases, a TMMP the present disclosure is administered intratumorally. In some cases, a TMMP of the present disclosure is administered peritumorally. In some cases, a TMMP of the present disclosure is administered intracranially. In some cases, a TMMP is administered subcutaneously. In some cases, a TMMP of the present disclosure is administered intralymphatically.

A TMMP of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be carried to effect systemic or local delivery of a TMMP of the present disclosure, a nucleic acid of the present disclosure, or a recombinant expression vector of the present disclosure. Where systemic delivery is desired, administration typically involves invasive or systemically absorbed topical or mucosal administration of pharmaceutical preparations.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment with a method of the present disclosure include individuals having a cancer in which the cancer cells overexpress MUC1, e.g., cancer cells that overexpress hypoglycosylated MUC1. Subjects suitable for treatment with a method of the present disclosure include individuals having a cancer such as multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach chancer; urothelial cancer; cervical cancer; and ovarian endometrial cancer.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure numbered 1-51 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory multimeric polypeptide comprising: at least one heterodimer comprising: a) a first polypeptide comprising: i) a MUC1 peptide comprising an epitope present in a mucin-1 (MUC1) antigen expressed on a cancer cell, wherein the MUC1 peptide has a length of at least 4 amino acids; and ii) first major histocompatibility complex (MHC) polypeptide; b) a second polypeptide comprising a second MHC polypeptide, and c) at least one immunomodulatory polypeptide, wherein the first and/or the second polypeptide comprises the immunomodulatory polypeptide.

Aspect 2. A T-cell modulatory multimeric polypeptide of claim 1, wherein at least one of the one or more immunomodulatory domains is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide, and wherein the MUC1 peptide and the first MHC polypeptide form a complex (MUC1 peptide/MHC complex) that binds to a T-cell receptor (TCR) on a T cell with an affinity of at least 10⁻⁷ M, such that:

i) the T-cell modulatory multimeric polypeptide binds to a first T cell with an affinity that is at least 25% higher than the affinity with which the T-cell modulatory multimeric polypeptide binds a second T cell, wherein the first T cell expresses on its surface the cognate co-immunomodulatory polypeptide and a TCR that binds the MUC1 peptide/MHC complex with an affinity of at least 10⁻⁷ M, and wherein the second T cell expresses on its surface the cognate co-immunomodulatory polypeptide but does not express on its surface a TCR that binds the MUC1 peptide/MHC complex with an affinity of at least 10⁻⁷ M; and/or

ii) the ratio of the binding affinity of a control T-cell modulatory multimeric polypeptide, wherein the control comprises a wild-type immunomodulatory polypeptide, to a cognate co-immunomodulatory polypeptide to the binding affinity of the T-cell modulatory multimeric polypeptide comprising a variant of the wild-type immunomodulatory polypeptide to the cognate co-immunomodulatory polypeptide, when measured by bio-layer interferometry, is in a range of from 1.5:1 to 106:1.

Aspect 3. A T-cell modulatory multimeric polypeptide of aspect 2, wherein: a) the T-cell modulatory multimeric polypeptide binds to the first T cell with an affinity that is at least 50%, at least 2-fold, at least 5-fold, or at least 10-fold higher than the affinity with which it binds the second T cell; and/or b) the variant immunomodulatory polypeptide binds the co-immunomodulatory polypeptide with an affinity of from about 10⁻⁴ M to about 10⁻⁷ M, from about 10⁻⁴ M to about 10⁻⁶ M, or from about 10⁻⁴ M to about 10⁻⁵ M; and/or c) wherein the ratio of the binding affinity of a control T-cell modulatory multimeric polypeptide, wherein the control comprises a wild-type immunomodulatory polypeptide, to a cognate co-immunomodulatory polypeptide to the binding affinity of the T-cell modulatory multimeric polypeptide comprising a variant of the wild-type immunomodulatory polypeptide to the cognate co-immunomodulatory polypeptide, when measured by bio-layer interferometry, is at least 10:1, at least 50:1, at least 10²:1, or at least 10³:1.

Aspect 4. A T-cell modulatory multimeric polypeptide of any one of aspects 1-3, wherein the first or the second polypeptide comprises an immunoglobulin (Ig) Fc polypeptide.

Aspect 5. A T-cell modulatory multimeric polypeptide of aspect 4, wherein the Ig Fc polypeptide is an IgG1 Fc polypeptide.

Aspect 6. A T-cell modulatory multimeric polypeptide of aspect 5, wherein IgG1 Fc polypeptide comprises one or more amino acid substitutions selected from N297A, L234A, L235A, L234F, L235E, and P331S.

Aspect 7. A T-cell modulatory multimeric polypeptide of any one of aspects 1-6, wherein:

a1) the first polypeptide comprises, in order from N-terminus to C-terminus: i) the MUC1 peptide; ii) the first MHC polypeptide; and iii) at least one immunomodulatory polypeptide; and b1) the second polypeptide comprises, in order from N-terminus to C-terminus: i) the second MHC polypeptide; and ii) an immunoglobulin (Ig) Fc polypeptide; or

a2) the first polypeptide comprises, in order from N-terminus to C-terminus: i) the MUC1 peptide; and ii) the first MHC polypeptide; and b2) the second polypeptide comprises, in order from N-terminus to C-terminus: i) at least one immunomodulatory polypeptide; ii) the second MHC polypeptide; and iii) an Ig Fc polypeptide; or

a3) the first polypeptide comprises, in order from N-terminus to C-terminus: i) the MUC1 peptide; and ii) the first MHC polypeptide; and b3) the second polypeptide comprises, in order from N-terminus to C-terminus: i) the second MHC polypeptide; and ii) an Ig Fc polypeptide; and iii) at least one immunomodulatory polypeptide; or

a4) the first polypeptide comprises, in order from N-terminus to C-terminus: i) the MUC1 peptide; and ii) the first MHC polypeptide; and b4) the second polypeptide comprises, in order from N-terminus to C-terminus: i) the second MHC polypeptide; and ii) at least one immunomodulatory polypeptide; or

a5) the first polypeptide comprises, in order from N-terminus to C-terminus: i) the MUC1 peptide; and ii) the first MHC polypeptide; and b5) a second polypeptide comprises, in order from N-terminus to C-terminus: i) at least one immunomodulatory polypeptide; and ii) the second MHC polypeptide; or

a6) the first polypeptide comprises, in order from N-terminus to C-terminus: i) the MUC1 peptide; ii) the first MHC polypeptide; and iii) at least one immunomodulatory polypeptide; and b6) the second polypeptide comprises: i) the second MHC polypeptide.

Aspect 8. A T-cell modulatory multimeric polypeptide of any one of aspects 1-7, wherein the first polypeptide comprises a peptide linker between the MUC1 peptide and the first MHC polypeptide and/or wherein the second polypeptide comprises a peptide linker between the immunomodulatory polypeptide and the second MHC polypeptide.

Aspect 9. A T-cell modulatory multimeric polypeptide of aspect 8, wherein the peptide linker comprises the amino acid sequence (GGGGS)n (SEQ ID NO: 254), where n is an integer from 1 to 10.

Aspect 10. A T-cell modulatory multimeric polypeptide of any one of aspects 1-9, wherein the first MHC polypeptide is a β2-microglobulin polypeptide; and wherein the second MHC polypeptide is an MHC class I heavy chain polypeptide.

Aspect 11. A T-cell modulatory multimeric polypeptide of any one of aspects 1-10, wherein the at least one immunomodulatory polypeptide is selected from the group consisting of a cytokine, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a PD-L2 polypeptide, and combinations thereof.

Aspect 12. A T-cell modulatory multimeric polypeptide of any one of aspects 1-11, wherein the at least one immunomodulatory polypeptide is an IL-2 polypeptide.

Aspect 13. A T-cell modulatory multimeric polypeptide of any one of aspects 1-12, wherein the multimeric polypeptide comprises at least two immunomodulatory polypeptides, and wherein at least two of the immunomodulatory polypeptides are the same.

Aspect 14. A T-cell modulatory multimeric polypeptide of aspect 13, wherein the 2 or more immunomodulatory polypeptides are in tandem.

Aspect 15. A T-cell modulatory multimeric polypeptide of any one of aspects 1-14, wherein the first polypeptide and the second polypeptide are covalently linked to one another.

Aspect 16. A T-cell modulatory multimeric polypeptide of aspect 15, wherein the covalent linkage is via a disulfide bond.

Aspect 17. A T-cell modulatory multimeric polypeptide of aspect 16, wherein the β2M polypeptide and the MHC heavy chain polypeptide are joined by a disulfide bond that joins a Cys residue in the β2M polypeptide and a Cys residue in the MHC heavy chain polypeptide.

Aspect 18. A T-cell modulatory multimeric polypeptide of aspect 17, wherein a Cys at amino acid residue 12 of the β2M polypeptide is disulfide bonded to a Cys at amino acid residue 236 of the MHC heavy chain polypeptide.

Aspect 19. A T-cell modulatory multimeric polypeptide of aspect 17, wherein the first polypeptide chain comprises a linker between the MUC1 peptide and the β2M polypeptide, and wherein the disulfide bond links a Cys present in the linker with a Cys of the MHC heavy chain polypeptide.

Aspect 20. A T-cell modulatory multimeric polypeptide of aspect 17, wherein the first polypeptide chain comprises a linker between the peptide epitope and the β2M polypeptide, and wherein the disulfide bond links a Cys substituted for Gly2 in the linker with a Cys substituted for Tyr84 of the MHC heavy chain polypeptide.

Aspect 21. A T-cell modulatory multimeric polypeptide of any one of aspects 1-14, wherein the first and the second polypeptides are covalently linked to one another via at least 2 disulfide bonds.

Aspect 22. The T-cell modulatory multimeric polypeptide of aspect 21, wherein: a) a first disulfide bond is between: i) a Cys present in a linker between the peptide epitope and the first MHC class I polypeptide, wherein the first MHC class I polypeptide is a β2M polypeptide; and ii) a Cys residue introduced via a Y84C substitution in the second MHC class I polypeptide, wherein the second MHC class I polypeptide is an MHC Class I heavy chain polypeptide; and b) a second disulfide bond is between: i) a Cys residue introduced into the β2M polypeptide via an R12C substitution; and ii) a Cys residue introduced into the MHC Class I heavy chain polypeptide via an A236C substitution.

Aspect 23. A T-cell modulatory multimeric polypeptide of aspect 22, wherein the linker comprises the amino acid sequence GCGGS (SEQ ID NO:139).

Aspect 24. A T-cell modulatory multimeric polypeptide of aspect 22, wherein the linker comprises the amino acid sequence GCGGS(GGGGS)n (SEQ ID NO: 140), where n is an integer from 1 to 10.

Aspect 25. A T-cell modulatory multimeric polypeptide of any one of aspects 1-24, wherein the MUC1 peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length).

Aspect 26. A T-cell modulatory multimeric polypeptide of any one of aspects 1-25, wherein the first or the second MHC polypeptide comprises: a) an amino acid sequence having at least 95% amino acid sequence identity to the HLA-A*0101, HLA-A*0201, HLA-A*0201, HLA-A*1101, HLA-A*2301, HLA-A*2402, HLA-A*2407, HLA-A*3303, or HLA-A*3401 amino acid sequence depicted in FIG. 7A; or b) an amino acid sequence having at least 95% amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A; or c) an amino acid sequence having at least 95% amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIG. 9A.

Aspect 27. A T-cell modulatory multimeric polypeptide of any one of aspects 1-25, wherein the first MHC polypeptide is a β2M polypeptide, and wherein the second MHC polypeptide comprises an amino acid sequence having at least 95% amino acid sequence identity to an HLA-A*0201 polypeptide.

Aspect 28. A T-cell modulatory multimeric polypeptide of any one of aspects 1-25, wherein the first MHC polypeptide is a β2M polypeptide, and wherein the second MHC polypeptide is an HLA-A*1101 polypeptide.

Aspect 29. A T-cell modulatory multimeric polypeptide of any one of aspects 1-25, wherein the first MHC polypeptide is a β2M polypeptide, and wherein the second MHC polypeptide comprises an amino acid sequence having at least 95% amino acid sequence identity to an HLA-A*3303 polypeptide.

Aspect 30. A T-cell modulatory multimeric polypeptide of any one of aspects 1-25, wherein the first MHC polypeptide is a β2M polypeptide, and wherein the second MHC polypeptide comprises an amino acid sequence having at least 95% amino acid sequence identity to an HLA-A*2401 polypeptide.

Aspect 31. A T-cell modulatory multimeric polypeptide of any one of aspects 1-30, wherein the immunomodulatory polypeptide is a variant IL-2 polypeptide comprising: i) an H16A substitution and an F42A substitution; or ii) an H16T substitution and an F42A substitution.

Aspect 32. A T-cell modulatory multimeric polypeptide of any one of aspects 1-31, wherein the MUC1 peptide has the amino acid sequence STAPPVHNV (SEQ ID NO:587) and has a length of 9 amino acids.

Aspect 33. A T-cell modulatory multimeric polypeptide of any one of aspects 1-31, wherein the MUC1 peptide has the amino acid sequence SLAPPVHNV (SEQ ID NO:588) and has a length of 9 amino acids.

Aspect 34. A T-cell modulatory multimeric polypeptide of any one of aspects 1-31, wherein the MUC1 peptide has the amino acid sequence STAPPAHGV (SEQ ID NO:586) and has a length of 9 amino acids.

Aspect 35. A T-cell modulatory multimeric polypeptide of any one of aspects 1-31, wherein the MUC1 peptide has the amino acid sequence SLAPPAHGV (SEQ ID NO:589) and has a length of 9 amino acids.

Aspect 36. A T-cell modulatory multimeric polypeptide of any one of aspects 1-35, wherein the first polypeptide and/or the second polypeptide comprises a tumor-targeting polypeptide (TTP).

Aspect 37. A T-cell modulatory multimeric polypeptide of aspect 36, wherein the TTP is an antibody specific for a cancer-associated antigen on the surface of a cancer cell.

Aspect 38. A T-cell modulatory multimeric polypeptide of aspect 37, wherein the antibody is a scFv or a nanobody.

Aspect 39. A T-cell modulatory multimeric polypeptide of any one of aspects 1-35, wherein the multimeric polypeptide comprises a first and a second heterodimer, and wherein the first and second heterodimers are covalently bound by one or more disulfide bonds between the Ig Fc polypeptides of the first and second heterodimers.

Aspect 40. A nucleic acid comprising a nucleotide sequence encoding a first or second polypeptide according to any one of aspects 1-39, wherein the first or second polypeptide comprises at least one immunomodulatory domain.

Aspect 41. An expression vector comprising the nucleic acid of aspect 40.

Aspect 42. A method of selectively modulating the activity of T cell specific for a peptide epitope, the method comprising contacting the T cell with a T-cell modulatory multimeric polypeptide according to any one of aspects 1-39, wherein said contacting selectively modulates the activity of the epitope-specific T cell.

Aspect 43. A method of treating a patient having a cancer, the method comprising administering to the patient an effective amount of a pharmaceutical composition comprising T-cell modulatory multimeric polypeptide according to any one of aspects 1-39.

Aspect 44. The method of aspect 43, wherein the cancer an adenocarcinoma or a hematological cancer.

Aspect 45. The method of aspect 44, wherein the cancer is a multiple myeloma.

Aspect 46. The method of aspect 44, wherein the cancer is a B-cell lymphoma.

Aspect 47. The method of any one of aspects 43-46, wherein said administering is intramuscular.

Aspect 48. The method of any one of aspects 43-46, wherein said administering is intravenous.

Aspect 49. A method of modulating an immune response in an individual, the method comprising administering to the individual an effective amount of the T-cell modulatory multimeric polypeptide of any one of aspects 1-39, wherein said administering induces an epitope-specific T cell response and an epitope-non-specific T cell response, wherein the ratio of the epitope-specific T cell response to the epitope-non-specific T cell response is at least 2:1.

Aspect 50. A method of delivering an immunomodulatory polypeptide selectively to a target T cell, the method comprising contacting a mixed population of T cells with a T-cell modulatory multimeric polypeptide of any one of aspects 1-39, wherein the mixed population of T cells comprises the target T cell and non-target T cells, wherein the target T cell is specific for the MUC1 epitope present within the T-cell modulatory multimeric polypeptide, and wherein said contacting delivers the one or more immunomodulatory polypeptides present within the T-cell modulatory multimeric polypeptide to the target T cell.

Aspect 51. A method of detecting, in a mixed population of T cells obtained from an individual, the presence of a target T cell that binds a MUC1 peptide, the method comprising: a) contacting in vitro the mixed population of T cells with the T-cell modulatory multimeric polypeptide of any one of aspects 1-39, wherein the T-cell modulatory multimeric polypeptide comprises the peptide epitope; and b) detecting activation and/or proliferation of T cells in response to said contacting, wherein activated and/or proliferated T cells indicates the presence of the target T cell.

### Aspects Set B

Aspects, including embodiments, of the present subject matter described above may be beneficial alone or in combination, with one or more other aspects or embodiments. Without limiting the foregoing description, certain non-limiting aspects of the disclosure numbered 1-49 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory multimeric polypeptide comprising at least one heterodimer comprising: a) a first polypeptide comprising: i) a peptide epitope comprising an amino acid sequence selected from the group consisting of: STAPPAHGV; STAPPVHNV; SLAPPVHNV; SLAPPAHGV; SAPDTRPAP; VTSAPDTRPAPGSTAPPAHG; PDTRPAPGSTAPPAHGVTSA; and LLLLTVLTV; and ii) first class I major histocompatibility complex (MHC) polypeptide; b) a second polypeptide comprising a second class I MHC polypeptide, and c) one or more immunomodulatory polypeptides, wherein the first and/or the second polypeptide comprises the one or more immunomodulatory polypeptides, and optionally wherein the first or the second polypeptide comprises an immunoglobulin (Ig) Fc polypeptide.

Aspect 2. A T-cell modulatory multimeric polypeptide of aspect 1, wherein at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide.

Aspect 3. A T-cell modulatory multimeric polypeptide of aspect 2, wherein the ratio of the binding affinity of the wild-type immunomodulatory polypeptide to a cognate co-immunomodulatory polypeptide to the binding affinity of the variant immunomodulatory polypeptide to the cognate co-immunomodulatory polypeptide, when measured by bio-layer interferometry, is at least 1.5:1.

Aspect 4. A T-cell modulatory multimeric polypeptide of aspect 2, wherein: the variant immunomodulatory polypeptide binds the co-immunomodulatory polypeptide with an affinity selected from the group consisting of from about 10⁻⁴ M to about 10⁻⁷ M, from about 10⁻⁴ M to about 10⁻⁶ M, or from about 10⁻⁴ M to about 10⁻⁵ M.

Aspect 5. A T-cell modulatory multimeric polypeptide of any one of aspects 1-4, wherein the first or the second polypeptide comprises an Ig Fc polypeptide, and wherein the Ig Fc polypeptide is an IgG1 Fc polypeptide.

Aspect 6. A T-cell modulatory multimeric polypeptide of aspect 5, wherein IgG1 Fc polypeptide comprises one or more amino acid substitutions selected from N297A, L234A, L235A, L234F, L235E, and P331S.

Aspect 7. A T-cell modulatory multimeric polypeptide of any one of aspects 1-6, wherein:

a1) the first polypeptide comprises, in order from N-terminus to C-terminus: i) the peptide epitope; and ii) the first MHC polypeptide; and b1) the second polypeptide comprises, in order from N-terminus to C-terminus: i) the one or more immunomodulatory polypeptides; ii) the second MHC polypeptide; and iii) an Ig Fc polypeptide; or

a2) the first polypeptide comprises, in order from N-terminus to C-terminus: i) the peptide epitope; and ii) the first MHC polypeptide; and b2) the second polypeptide comprises, in order from N-terminus to C-terminus: i) the second MHC polypeptide; and ii) the one or more immunomodulatory polypeptides; and iii) an Ig Fc polypeptide; or

a3) the first polypeptide comprises, in order from N-terminus to C-terminus: i) the peptide epitope; and ii) the first MHC polypeptide; and b3) the second polypeptide comprises, in order from N-terminus to C-terminus:i) the second MHC polypeptide; and ii) an Ig Fc polypeptide; and iii) the one or more immunomodulatory polypeptides; or

a4) the first polypeptide comprises, in order from N-terminus to C-terminus: i) the one or more immunomodulatory polypeptides; ii) the peptide epitope; and iii) the first MHC polypeptide; and b4) the second polypeptide comprises, in order from N-terminus to C-terminus: i) the second MHC polypeptide; and ii) an Ig Fc polypeptide; or

a5) the first polypeptide comprises, in order from N-terminus to C-terminus: i) the peptide epitope; ii) the first MHC polypeptide; and iii) the one or more immunomodulatory polypeptides; and b5) the second polypeptide comprises, in order from N-terminus to C-terminus: i) the second MHC polypeptide; and ii) an immunoglobulin (Ig) Fc polypeptide.

Aspect 8. A T-cell modulatory multimeric polypeptide of any one of aspects 1-7, wherein the first MHC polypeptide is a β2-microglobulin polypeptide; and wherein the second MHC polypeptide is an MHC class I heavy chain polypeptide.

Aspect 9. A T-cell modulatory multimeric polypeptide of any one of aspects 1-8, wherein at least one of the one or more immunomodulatory polypeptides is a stimulatory polypeptide.

Aspect 10. A T-cell modulatory multimeric polypeptide of aspect 9, wherein the stimulatory polypeptide is selected from the group consisting of a 4-1BBL, ICOS-L, OX-40L, CD80, CD86, CD70, a cytokine, and variants and combinations thereof.

Aspect 11. A T-cell modulatory multimeric polypeptide of any one of aspects 9 and 10, wherein at least one of the one or more immunomodulatory polypeptides is an IL-2 polypeptide.

Aspect 12. A T-cell modulatory multimeric polypeptide of aspect 11, wherein the immunomodulatory polypeptide is a variant IL-2 polypeptide.

Aspect 13. A T-cell modulatory multimeric polypeptide of aspect 12, wherein the variant IL-2 polypeptide comprises: i) an H16A substitution and an F42A substitution; or ii) an H16T substitution and an F42A substitution.

Aspect 14. A T-cell modulatory multimeric polypeptide of any one of aspects 1-8, wherein at least one of the one or more immunomodulatory polypeptides is an inhibitory polypeptide, optionally wherein the inhibitory polypeptide is selected from the group consisting of PD-L1, FasL, a cytokine, and PD-L2, and variants and combinations thereof.

Aspect 15. A T-cell modulatory multimeric polypeptide of any one of aspects 1-14, wherein the multimeric polypeptide comprises two or more immunomodulatory polypeptides, and wherein at least two of the immunomodulatory polypeptides are the same.

Aspect 16. A T-cell modulatory multimeric polypeptide of aspect 15, wherein the two or more immunomodulatory polypeptides are in tandem.

Aspect 17. A T-cell modulatory multimeric polypeptide of any of aspects 1-6, wherein: a1) the first polypeptide comprises, in order from N-terminus to C-terminus: i) the peptide epitope; and ii) the first MHC polypeptide; and b1) the second polypeptide comprises, in order from N-terminus to C-terminus: i) the one or more immunomodulatory polypeptides; ii) the second MHC polypeptide; and iii) an immunoglobulin (Ig) Fc polypeptide.

Aspect 18. A T-cell modulatory multimeric polypeptide of any of aspects 1-6, wherein: a3) the first polypeptide comprises, in order from N-terminus to C-terminus: i) the peptide epitope; and ii) the first MHC polypeptide; and b3) the second polypeptide comprises, in order from N-terminus to C-terminus: i) the second MHC polypeptide; and ii) an Ig Fc polypeptide; and iii) the one or more immunomodulatory polypeptides.

Aspect 19. A T-cell modulatory multimeric polypeptide of any one of aspects 1-18, wherein the first polypeptide and the second polypeptide are covalently linked to one another, optionally wherein the covalent linkage is via a disulfide bond.

Aspect 20. A T-cell modulatory multimeric polypeptide of aspect 19, wherein the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide.

Aspect 21. A T-cell modulatory multimeric polypeptide of aspect 19, wherein the first polypeptide chain comprises a linker between the peptide epitope and the β2M polypeptide, and wherein the disulfide bond links a Cys present in the linker with a Cys of the MHC heavy chain polypeptide.

Aspect 22. A T-cell modulatory multimeric polypeptide of aspect 19, wherein the first polypeptide chain comprises a linker between the peptide epitope and the β2M polypeptide, and wherein the disulfide bond links a Cys substituted for Gly2 in the linker with a Cys substituted for Tyr84 of the MHC heavy chain polypeptide.

Aspect 23. A T-cell modulatory multimeric polypeptide of any one of aspects 1-18, wherein the first and the second polypeptides are covalently linked to one another via at least 2 disulfide bonds.

Aspect 24. A T-cell modulatory multimeric polypeptide of aspect 23, wherein: a) a first disulfide bond is between: i) a Cys present in a linker between the peptide epitope and the first MHC class I polypeptide, wherein the first MHC class I polypeptide is a β2M polypeptide; and ii) a Cys residue introduced via a Y84C substitution in the second MHC class I polypeptide, wherein the second MHC class I polypeptide is an MHC Class I heavy chain polypeptide; and b) a second disulfide bond is between: i) a Cys residue introduced into the β2M polypeptide via an R12C substitution; and ii) a Cys residue introduced into the MHC Class I heavy chain polypeptide via an A236C substitution.

Aspect 25. A T-cell modulatory multimeric polypeptide of aspect 24, wherein the linker comprises the amino acid sequence GCGGS.

Aspect 26. A T-cell modulatory multimeric polypeptide of aspect 24, wherein the linker comprises the amino acid sequence GCGGS(GGGGS)n, where n is an integer from 1 to 10.

Aspect 27. A T-cell modulatory multimeric polypeptide of any one of aspects 1-26, wherein the peptide epitope is a peptide of at least 9 amino acids in length, e.g., from 9 amino acids to about 25 amino acids (e.g., 10 amino acids (aa), 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 9 to 20 amino acids, from 9 to 18 amino acids, from 9 to 15 amino acids, from 9 to 12 amino acids, from 9 to 10 amino acids, from 10 to 20 amino acids, and from 15 to 25 amino acids in length).

Aspect 28. A T-cell modulatory multimeric polypeptide of any one of aspects 1-27, wherein the first or the second MHC polypeptide comprises: a) an amino acid sequence having at least 95% amino acid sequence identity to the HLA-A*0101, HLA-A*0201, HLA-A*0201, HLA-A*1101, HLA-A*2301, HLA-A*2402, HLA-A*2407, HLA-A*3303, or HLA-A*3401 amino acid sequence depicted in FIG. 7A; or b) an amino acid sequence having at least 95% amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A; or c) an amino acid sequence having at least 95% amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIG. 9A.

Aspect 29. A T-cell modulatory multimeric polypeptide of any one of aspects 1-27, wherein the first MHC polypeptide is a β2M polypeptide, and wherein the second MHC polypeptide comprises an amino acid sequence having at least 95% amino acid sequence identity to an HLA-A*0201 polypeptide.

Aspect 30. A T-cell modulatory multimeric polypeptide of any one of aspects 1-27, wherein the first MHC polypeptide is a β2M polypeptide, and wherein the second MHC polypeptide is an HLA-A*1101 polypeptide.

Aspect 31. A T-cell modulatory multimeric polypeptide of any one of aspects 1-27, wherein the first MHC polypeptide is a β2M polypeptide, and wherein the second MHC polypeptide comprises an amino acid sequence having at least 95% amino acid sequence identity to an HLA-A*3303 polypeptide.

Aspect 32. A T-cell modulatory multimeric polypeptide of any one of aspects 1-27, wherein the first MHC polypeptide is a β2M polypeptide, and wherein the second MHC polypeptide comprises an amino acid sequence having at least 95% amino acid sequence identity to an HLA-A*2401 polypeptide.

Aspect 33. A T-cell modulatory multimeric polypeptide of any one of aspects 1-32, wherein the peptide epitope comprises the amino acid sequence STAPPVHNV and has a length of 9 amino acids.

Aspect 34. A T-cell modulatory multimeric polypeptide of any one of aspects 1-32, wherein the peptide epitope comprises the amino acid sequence SLAPPVHNV and has a length of 9 amino acids.

Aspect 35. A T-cell modulatory multimeric polypeptide of any one of aspects 1-32, wherein the peptide epitope comprising the amino acid sequence STAPPAHGV and has a length of 9 amino acids.

Aspect 36. A T-cell modulatory multimeric polypeptide of any one of aspects 1-32, wherein the peptide epitope comprising the amino acid sequence SLAPPAHGV and has a length of 9 amino acids.

Aspect 37. A T-cell modulatory multimeric polypeptide of any one of aspects 1-36, wherein the first polypeptide and/or the second polypeptide comprises a tumor-targeting polypeptide (TTP).

Aspect 38. A T-cell modulatory multimeric polypeptide of aspect 37, wherein the TTP is an antibody specific for a cancer-associated antigen on the surface of a cancer cell.

Aspect 39. A T-cell modulatory multimeric polypeptide of aspect 38, wherein the antibody is a scFv or a nanobody.

Aspect 40. A T-cell modulatory multimeric polypeptide of any one of aspects 1-37, wherein the multimeric polypeptide comprises a first and a second heterodimer, and wherein the first and second heterodimers are covalently bound by one or more disulfide bonds between the Ig Fc polypeptides of the first and second heterodimers.

Aspect 41. A nucleic acid comprising a nucleotide sequence encoding a first or second polypeptide according to any one of aspects 1-40.

Aspect 42. An expression vector comprising the nucleic acid of aspect 41.

Aspect 43. A method of selectively modulating the activity of T cell specific for a peptide epitope, the method comprising contacting the T cell with a T-cell modulatory multimeric polypeptide according to any one of aspects 1-40, wherein said contacting selectively modulates the activity of the epitope-specific T cell.

Aspect 44. A method of treating a patient having a cancer, the method comprising administering to the patient an effective amount of a pharmaceutical composition comprising a T-cell modulatory multimeric polypeptide of any one of aspects 1-40.

Aspect 45. The method of aspect 44, wherein the cancer is selected from the group consisting of an adenocarcinoma, a hematological cancer, a multiple myeloma, a B-cell lymphoma,

Aspect 46. The method of aspect 44 or aspect 45, wherein said administering is subcutaneously, intraperitoneally, intramuscularly, intradermal, intratumoral, peritumoral, and/or intravenously.

Aspect 47. A method of modulating an immune response in an individual, the method comprising administering to the individual an effective amount of a T-cell modulatory multimeric polypeptide of any one of aspects 1-40, wherein said administering induces an epitope-specific T cell response and an epitope-non-specific T cell response, wherein the ratio of the epitope-specific T cell response to the epitope-non-specific T cell response is at least 2:1.

Aspect 48. A method of delivering an immunomodulatory polypeptide selectively to a target T cell, the method comprising contacting a mixed population of T cells with a T-cell modulatory multimeric polypeptide of any one of aspects 1-40, wherein the mixed population of T cells comprises the target T cell and non-target T cells, wherein the target T cell is specific for the MUC1 epitope present within the T-cell modulatory multimeric polypeptide, and wherein said contacting delivers the one or more immunomodulatory polypeptides present within the T-cell modulatory multimeric polypeptide to the target T cell.

Aspect 49. A method of detecting, in a mixed population of T cells obtained from an individual, the presence of a target T cell that binds a peptide epitope, the method comprising: a) contacting in vitro the mixed population of T cells with a T-cell modulatory multimeric polypeptide of any one of aspects 1-40, wherein the T-cell modulatory multimeric polypeptide comprises the peptide epitope; and b) detecting activation and/or proliferation of T cells in response to said contacting, wherein activated and/or proliferated T cells indicates the presence of the target T cell.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A T-cell modulatory multimeric polypeptide comprising:
at least one heterodimer comprising:
a) a first polypeptide comprising:
i) a peptide epitope comprising an amino acid sequence selected from the group consisting of: STAPPAHGV; STAPPVHNV; SLAPPVHNV; SLAPPAHGV; SAPDTRPAP; VTSAPDTRPAPGSTAPPAHG; PDTRPAPGSTAPPAHGVTSA; and LLLLTVLTV; and
ii) first class I major histocompatibility complex (MHC) polypeptide;
b) a second polypeptide comprising a second class I MHC polypeptide, and
c) one or more immunomodulatory polypeptides,
wherein the first and/or the second polypeptide comprises the one or more immunomodulatory polypeptides,
and optionally wherein the first or the second polypeptide comprises an immunoglobulin (Ig) Fc polypeptide.

2. A T-cell modulatory multimeric polypeptide of claim 1, wherein at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide.

3. A T-cell modulatory multimeric polypeptide of claim 2, wherein the ratio of the binding affinity of the wild-type immunomodulatory polypeptide to a cognate co-immunomodulatory polypeptide to the binding affinity of the variant immunomodulatory polypeptide to the cognate co-immunomodulatory polypeptide, when measured by bio-layer interferometry, is at least 1.5:1.

4. A T-cell modulatory multimeric polypeptide of claim 2, wherein:
the variant immunomodulatory polypeptide binds the co-immunomodulatory polypeptide with an affinity selected from the group consisting of from about 10⁻⁴ M to about 10⁻⁷ M, from about 10⁻⁴ M to about 10⁻⁶ M, or from about 10⁻⁴ M to about 10⁻⁵ M.

5. A T-cell modulatory multimeric polypeptide of any one of claims 1-4, wherein the first or the second polypeptide comprises an Ig Fc polypeptide, and wherein the Ig Fc polypeptide is an IgG1 Fc polypeptide.

6. A T-cell modulatory multimeric polypeptide of claim 5, wherein IgG1 Fc polypeptide comprises one or more amino acid substitutions selected from N297A, L234A, L235A, L234F, L235E, and P331S.

7. A T-cell modulatory multimeric polypeptide of any one of claims 1-6, wherein
a1) the first polypeptide comprises, in order from N-terminus to C-terminus:
i) the peptide epitope; and
ii) the first MHC polypeptide; and
b1) the second polypeptide comprises, in order from N-terminus to C-terminus:
i) the one or more immunomodulatory polypeptides;
ii) the second MHC polypeptide; and
iii) an Ig Fc polypeptide; or
a2) the first polypeptide comprises, in order from N-terminus to C-terminus:
i) the peptide epitope; and
ii) the first MHC polypeptide; and
b2) the second polypeptide comprises, in order from N-terminus to C-terminus:
i) the second MHC polypeptide; and
ii) the one or more immunomodulatory polypeptides; and
iii) an Ig Fc polypeptide; or
a3) the first polypeptide comprises, in order from N-terminus to C-terminus:
i) the peptide epitope; and
ii) the first MHC polypeptide; and
b3) the second polypeptide comprises, in order from N-terminus to C-terminus:
i) the second MHC polypeptide; and
ii) an Ig Fc polypeptide; and
iii) the one or more immunomodulatory polypeptides; or
a4) the first polypeptide comprises, in order from N-terminus to C-terminus:
i) the one or more immunomodulatory polypeptides;
ii) the peptide epitope; and
iii) the first MHC polypeptide; and
b4) the second polypeptide comprises, in order from N-terminus to C-terminus:
i) the second MHC polypeptide; and
ii) an Ig Fc polypeptide; or
a5) the first polypeptide comprises, in order from N-terminus to C-terminus:
i) the peptide epitope;
ii) the first MHC polypeptide; and
iii) the one or more immunomodulatory polypeptides; and
b5) the second polypeptide comprises, in order from N-terminus to C-terminus:
i) the second MHC polypeptide; and
ii) an immunoglobulin (Ig) Fc polypeptide.

8. A T-cell modulatory multimeric polypeptide of any one of claims 1-7, wherein the first MHC polypeptide is a β2-microglobulin polypeptide; and wherein the second MHC polypeptide is an MHC class I heavy chain polypeptide.

9. A T-cell modulatory multimeric polypeptide of any one of claims 1-8, wherein at least one of the one or more immunomodulatory polypeptides is a stimulatory polypeptide.

10. A T-cell modulatory multimeric polypeptide of claim 9, wherein the stimulatory polypeptide is selected from the group consisting of a 4-1BBL, ICOS-L, OX-40L, CD80, CD86, CD70, a cytokine, and variants and combinations thereof.

11. A T-cell modulatory multimeric polypeptide of any one of claims 9 and 10, wherein at least one of the one or more immunomodulatory polypeptides is an IL-2 polypeptide.

12. A T-cell modulatory multimeric polypeptide of claim 11, wherein the immunomodulatory polypeptide is a variant IL-2 polypeptide.

13. A T-cell modulatory multimeric polypeptide of claim 12, wherein the variant IL-2 polypeptide comprises: i) an H16A substitution and an F42A substitution; or ii) an H16T substitution and an F42A substitution.

14. A T-cell modulatory multimeric polypeptide of any one of claims 1-8, wherein at least one of the one or more immunomodulatory polypeptides is an inhibitory polypeptide, optionally wherein the inhibitory polypeptide is selected from the group consisting of PD-L1, FasL, a cytokine, and PD-L2, and variants and combinations thereof.

15. A T-cell modulatory multimeric polypeptide of any one of claims 1-14, wherein the multimeric polypeptide comprises two or more immunomodulatory polypeptides, and wherein at least two of the immunomodulatory polypeptides are the same.

16. A T-cell modulatory multimeric polypeptide of claim 15, wherein the two or more immunomodulatory polypeptides are in tandem.

17. A T-cell modulatory multimeric polypeptide of any of claims 1-6, wherein:
a1) the first polypeptide comprises, in order from N-terminus to C-terminus:
i) the peptide epitope; and
ii) the first MHC polypeptide; and
b1) the second polypeptide comprises, in order from N-terminus to C-terminus:
i) the one or more immunomodulatory polypeptides;
ii) the second MHC polypeptide; and
iii) an immunoglobulin (Ig) Fc polypeptide.

18. A T-cell modulatory multimeric polypeptide of any of claims 1-6, wherein:
a3) the first polypeptide comprises, in order from N-terminus to C-terminus:
i) the peptide epitope; and
ii) the first MHC polypeptide; and
b3) the second polypeptide comprises, in order from N-terminus to C-terminus:
i) the second MHC polypeptide; and
ii) an Ig Fc polypeptide; and
iii) the one or more immunomodulatory polypeptides.

19. A T-cell modulatory multimeric polypeptide of any one of claims 1-18, wherein the first polypeptide and the second polypeptide are covalently linked to one another, optionally wherein the covalent linkage is via a disulfide bond.

20. A T-cell modulatory multimeric polypeptide of claim 19, wherein the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide.

21. A T-cell modulatory multimeric polypeptide of claim 19, wherein the first polypeptide chain comprises a linker between the peptide epitope and the β2M polypeptide, and wherein the disulfide bond links a Cys present in the linker with a Cys of the MHC heavy chain polypeptide.

22. A T-cell modulatory multimeric polypeptide of claim 19, wherein the first polypeptide chain comprises a linker between the peptide epitope and the β2M polypeptide, and wherein the disulfide bond links a Cys substituted for Gly2 in the linker with a Cys substituted for Tyr84 of the MHC heavy chain polypeptide.

23. A T-cell modulatory multimeric polypeptide of any one of claims 1-18, wherein the first and the second polypeptides are covalently linked to one another via at least 2 disulfide bonds.

24. A T-cell modulatory multimeric polypeptide of claim 23, wherein:
a) a first disulfide bond is between: i) a Cys present in a linker between the peptide epitope and the first MHC class I polypeptide, wherein the first MHC class I polypeptide is a β2M polypeptide; and ii) a Cys residue introduced via a Y84C substitution in the second MHC class I polypeptide, wherein the second MHC class I polypeptide is an MHC Class I heavy chain polypeptide; and
b) a second disulfide bond is between: i) a Cys residue introduced into the β2M polypeptide via an R12C substitution; and ii) a Cys residue introduced into the MHC Class I heavy chain polypeptide via an A236C substitution.

25. A T-cell modulatory multimeric polypeptide of claim 24, wherein the linker comprises the amino acid sequence GCGGS.

26. A T-cell modulatory multimeric polypeptide of claim 24, wherein the linker comprises the amino acid sequence GCGGS(GGGGS)n, where n is an integer from 1 to 10.

27. A T-cell modulatory multimeric polypeptide of any one of claims 1-26, wherein the peptide epitope is a peptide of at least 9 amino acids in length, e.g., from 9 amino acids to about 25 amino acids (e.g., 10 amino acids (aa), 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 9 to 20 amino acids, from 9 to 18 amino acids, from 9 to 15 amino acids, from 9 to 12 amino acids, from 9 to 10 amino acids, from 10 to 20 amino acids, and from 15 to 25 amino acids in length).

28. A T-cell modulatory multimeric polypeptide of any one of claims 1-27, wherein the first or the second MHC polypeptide comprises:
a) an amino acid sequence having at least 95% amino acid sequence identity to the HLA-A*0101, HLA-A*0201, HLA-A*0201, HLA-A*1101, HLA-A*2301, HLA-A*2402, HLA-A*2407, HLA-A*3303, or HLA-A*3401 amino acid sequence depicted in FIG. 7A; or
b) an amino acid sequence having at least 95% amino acid sequence identity to the HLA-B*0702, HLA-B*0801, HLA-B*1502, HLA-B*3802, HLA-B*4001, HLA-B*4601, or HLA-B*5301 amino acid sequence depicted in FIG. 8A; or
c) an amino acid sequence having at least 95% amino acid sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIG. 9A.

29. A T-cell modulatory multimeric polypeptide of any one of claims 1-27, wherein the first MHC polypeptide is a β2M polypeptide, and wherein the second MHC polypeptide comprises an amino acid sequence having at least 95% amino acid sequence identity to an HLA-A*0201 polypeptide.

30. A T-cell modulatory multimeric polypeptide of any one of claims 1-27, wherein the first MHC polypeptide is a β2M polypeptide, and wherein the second MHC polypeptide is an HLA-A*1101 polypeptide.

31. A T-cell modulatory multimeric polypeptide of any one of claims 1-27, wherein the first MHC polypeptide is a β2M polypeptide, and wherein the second MHC polypeptide comprises an amino acid sequence having at least 95% amino acid sequence identity to an HLA-A*3303 polypeptide.

32. A T-cell modulatory multimeric polypeptide of any one of claims 1-27, wherein the first MHC polypeptide is a β2M polypeptide, and wherein the second MHC polypeptide comprises an amino acid sequence having at least 95% amino acid sequence identity to an HLA-A*2401 polypeptide.

33. A T-cell modulatory multimeric polypeptide of any one of claims 1-32, wherein the peptide epitope comprises the amino acid sequence STAPPVHNV and has a length of 9 amino acids.

34. A T-cell modulatory multimeric polypeptide of any one of claims 1-32, wherein the peptide epitope comprises the amino acid sequence SLAPPVHNV and has a length of 9 amino acids.

35. A T-cell modulatory multimeric polypeptide of any one of claims 1-32, wherein the peptide epitope comprising the amino acid sequence STAPPAHGV and has a length of 9 amino acids.

36. A T-cell modulatory multimeric polypeptide of any one of claims 1-32, wherein the peptide epitope comprising the amino acid sequence SLAPPAHGV and has a length of 9 amino acids.

37. A T-cell modulatory multimeric polypeptide of any one of claims 1-36, wherein the first polypeptide and/or the second polypeptide comprises a tumor-targeting polypeptide (TTP).

38. A T-cell modulatory multimeric polypeptide of claim 37, wherein the TTP is an antibody specific for a cancer-associated antigen on the surface of a cancer cell.

39. A T-cell modulatory multimeric polypeptide of claim 38, wherein the antibody is a scFv or a nanobody.

40. A T-cell modulatory multimeric polypeptide of any one of claims 1-37, wherein the multimeric polypeptide comprises a first and a second heterodimer, and
wherein the first and second heterodimers are covalently bound by one or more disulfide bonds between the Ig Fc polypeptides of the first and second heterodimers.

41. A nucleic acid comprising a nucleotide sequence encoding a first or second polypeptide according to any one of claims 1-40.

42. An expression vector comprising the nucleic acid of claim 41.

43. A method of selectively modulating the activity of T cell specific for a peptide epitope, the method comprising contacting the T cell with a T-cell modulatory multimeric polypeptide according to any one of claims 1-40, wherein said contacting selectively modulates the activity of the epitope-specific T cell.

44. A method of treating a patient having a cancer, the method comprising administering to the patient an effective amount of a pharmaceutical composition comprising a T-cell modulatory multimeric polypeptide of any one of claims 1-40.

45. The method of claim 44, wherein the cancer is selected from the group consisting of an adenocarcinoma, a hematological cancer, a multiple myeloma, a B-cell lymphoma,

46. The method of claim 44 or claim 45, wherein said administering is subcutaneously, intraperitoneally, intramuscularly, intradermal, intratumoral, peritumoral, and/or intravenously.

47. A method of modulating an immune response in an individual, the method comprising administering to the individual an effective amount of a T-cell modulatory multimeric polypeptide of any one of claims 1-40,
wherein said administering induces an epitope-specific T cell response and an epitope-non-specific T cell response,
wherein the ratio of the epitope-specific T cell response to the epitope-non-specific T cell response is at least 2:1.

48. A method of delivering an immunomodulatory polypeptide selectively to a target T cell, the method comprising contacting a mixed population of T cells with a T-cell modulatory multimeric polypeptide of any one of claims 1-40, wherein the mixed population of T cells comprises the target T cell and non-target T cells, wherein the target T cell is specific for the MUC1 epitope present within the T-cell modulatory multimeric polypeptide, and wherein said contacting delivers the one or more immunomodulatory polypeptides present within the T-cell modulatory multimeric polypeptide to the target T cell.

49. A method of detecting, in a mixed population of T cells obtained from an individual, the presence of a target T cell that binds a peptide epitope, the method comprising:
a) contacting in vitro the mixed population of T cells with a T-cell modulatory multimeric polypeptide of any one of claims 1-40, wherein the T-cell modulatory multimeric polypeptide comprises the peptide epitope; and
b) detecting activation and/or proliferation of T cells in response to said contacting, wherein activated and/or proliferated T cells indicates the presence of the target T cell.
